(19) United States
(12) Patent Application Publication
(10) Pub. No.: US 2021/0130346 A1
(43) Pub. Date: May 6, 2021

(54) Bromodomain Inhibitors

(71) Applicant: AbbVie Inc., North Chicago, IL (US)

(72) Inventors: David Hardee, Vernon Hills, IL (US); Jason Brewer, Antioch, IL (US); Lisa Hasvold, Grayslake, IL (US); Dachun Liu, Vernon Hills, IL (US); Keith McDaniel, Wauconda, IL (US); Michael Schrimpf, Grayslake, IL (US); George Sheppard, Wilmette, IL (US)

(73) Assignee: AbbVie Inc., North Chicago, IL (US)

(21) Appl. No.: 16/605,039

(22) PCT Filed: Apr. 14, 2017
(86) PCT No.: PCT/CN2017/080512

§ 371 Date: Oct. 14, 2019

(51) Int. Cl. C07D 471/04; A61P 35/04

(52) U.S. Cl. CPC C07D 471/04; A61P 35/04

(57) ABSTRACT

Compounds of formula (I), wherein R¹, R², R³, R⁵, R⁶, R⁷, A¹, A², A³, A⁴, X¹, and X² have any of the values defined in the specification and pharmaceutically acceptable salts thereof, which are useful as agents in the treatment of diseases and conditions, including inflammatory diseases, cancer, and AIDS are provided. Pharmaceutical compositions comprising of compounds of formula (I) are also provided.

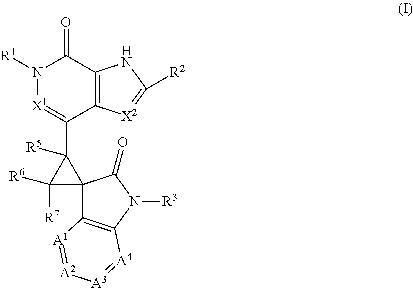

### BACKGROUND

Bromodomains refer to conserved protein structural folds which bind to N-acetylated lysine residues that are found in some proteins. The BET family of bromodomain containing proteins comprises four members (BRD2, BRD3, BRD4 and BRDt). Each member of the BET family employs two bromodomains to recognize N-acetylated lysine residues typically, but not exclusively those found on transcription factors (Shi, J., et al. Cancer Cell 25(2): 210-225 (2014)) or on the amino-terminal tails of histone proteins. These interactions modulate gene expression by recruiting transcription factors to specific genome locations within chromatin. For example, histone-bound BRD4 recruits the transcription factor P-TEFb to promoters, resulting in the expression of a subset of genes involved in cell cycle progression (Yang et al., Mol. Cell. Biol. 28: 967-976 (2008)). BRD2 and BRD3 also function as transcriptional regulators of growth promoting genes (LeRoy et al., Mol. Cell 30: 51-60 (2008)). BET family members were recently established as being important for the maintenance of several cancer types (Zuber et al., Nature 478: 524-528 (2011); Mertz et al; Proc. Nat'l. Acad. Sci. 108: 16669-16674 (2011); Delmore et al., Cell 146: 1-14, (2011); Dawson et al., Nature 478: 529-533 (2011)). BET family members have also been implicated in mediating acute inflammatory responses through the canonical NF-KB pathway (Huang et al., Mol. Cell. Biol. 29: 1375-1387 (2009)) resulting in the upregulation of genes associated with the production of cytokines (Nicodeme et al., Nature 468: 1119-1123, (2010)). Suppression of cytokine induction by BET bromodomain inhibitors has been shown to be an effective approach to treat inflammation-mediated kidney disease in an animal model (Zhang, et al., J. Biol. Chem. 287: 28840-28851 (2012)). BRD2 function has been linked to pre-disposition for dyslipidemia or improper regulation of adipogenesis, elevated inflammatory profiles and increased susceptibility to autoimmune diseases (Denis, Discovery Medicine 10: 489-499 (2010)). The human immunodeficiency virus utilizes BRD4 to initiate transcription of viral RNA from stably integrated viral DNA (Jang et al., Mol. Cell, 19: 523-534 (2005)). BET bromodomain inhibitors have also been shown to reactivate HIV transcription in models of latent T cell infection and latent monocyte infection (Banerjee, et al, J. Leukocyte Biol. doi:10.1189/jlb.0312165). BRDt has an important role in spermatogenesis that is blocked by BET bromodomain inhibitors (Matzuk, et al., Cell 150: 673-684 (2012)). Thus, compounds that inhibit the binding of BET family bromodomains to their cognate acetylated lysine proteins are being pursued for the treatment of cancer, inflammatory diseases, kidney diseases, diseases involving metabolism or fat accumulation, and some viral infections, as well as for providing a method for male contraception. Accordingly, there is an ongoing medical need to develop new drugs to treat these indications.

### SUMMARY

In one aspect the present invention provides for compounds of formula (I) or a pharmaceutically acceptable salt thereof,

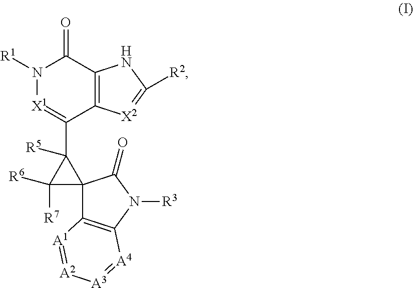

wherein
X¹ is N or C(R^{x1}) wherein R^{x1} is hydrogen, halogen, C₁-C₃ alkyl, or C₁-C₃ haloalkyl;
X² is N or CH;
R¹ is C₁-C₃ alkyl;
R² is hydrogen, G^{x1}, —C(O)OH, —NHC(O)R^{A}, or —C(O)N(H)R^{A1}; wherein
   R^{A} is G^{x2}, C₁-C₆ haloalkyl, or C₁-C₆ alkyl; wherein the C₁-C₆ haloalkyl and the C₁-C₆ alkyl are optionally substituted with 1 or 2 substituents independently selected from the group consisting of —OR^{B}, —CN, G^{x2}, and —N(R^{B})₂;
   R^{A1} is hydrogen, G^{x2}, C₁-C₆ haloalkyl, or C₁-C₆ alkyl; wherein the C₁-C₆ haloalkyl and the C₁-C₆ alkyl are optionally substituted with 1 or 2 substituents independently selected from the group consisting of —OR^{B}, —CN, G^{x2}, and —N(R^{B})₂;
   G^{x1} and G^{x2}, at each occurrence, are each independently C₆-C₁₀ aryl, 5-11 membered heteroaryl, C₃-C₇ monocyclic cycloalkyl, C₄-C₆ monocyclic cycloalkenyl, or 4-11 membered heterocycle; each G^{x1} and G^{x2} are optionally substituted with 1, 2, or 3 substituents independently selected from the group consisting of cyclopropyl and R^{s}; wherein the cyclopropyl is optionally substituted with 1, 2, or 3 substituents independently selected from the group consisting of halogen, C₁-C₆ haloalkyl, and C₁-C₆ alkyl;
   each R^{B} is independently hydrogen, C₁-C₆ alkyl, or C₁-C₆ haloalkyl;
R³ is hydrogen, C₂-C₆ alkenyl, C₂-C₆ alkynyl, C₃-C₇ monocyclic cycloalkyl, C₁-C₆ alkyl, or C₁-C₆ haloalkyl, wherein the C₁-C₆ alkyl and the C₁-C₆ haloalkyl are each optionally substituted with 1 or 2 substituents independently selected from the group consisting of —OR^{a}, —CN, —N(R^{a})₂, and phenyl; wherein the C₃-C₇ monocyclic cycloalkyl and the phenyl are each optionally substituted with 1, 2, or 3 independently selected R^{t} groups;
R^{a}, at each occurrence, is independently hydrogen, C₁-C₆ alkyl, or C₁-C₆ haloalkyl;
A¹, A², A³, and A⁴ are CR⁴ or
one or two of A¹, A², A³, and A⁴ are N, and the others are CR⁴;
R⁴, at each occurrence, is independently hydrogen, C₁-C₆ alkyl, C₂-C₆ alkenyl, C₂-C₆ alkynyl, halogen, C₁-C₆ haloalkyl, —CN,
   NO₂, —OR^{x}, —OC(O)R^{y}, —OC(O)N(R^{x})₂, —S(O)₂R^{x}, —S(O)₂N(R^{x})₂, —C(O)R^{x}, —C(O)OR^{x}, —C(O)N(R^{x})₂, —C(O)N(R^{x})S(O)₂R^{y}, —N(R^{x})₂, —N(R^{x})C(O)R^{y}, —N(R^{x})S(O)₂R^{y}, —N(R^{x})C(O)O(R^{y}), —N(R^{x})C(O)N(R^{x})₂, —N(R^{x})S(O)₂N(R^{x})₂, —(C₁-C₆ alkylenyl)-CN, —(C₁-C₆ alkylenyl)—OR^{x}, —(C₁-C₆ alkylenyl)—OC(O)R^{y}, —(C₁-C₆ alkylenyl)—OC(O)N(R^{x})₂, —(C₁-C₆ alkylenyl)-SR^{x}, —(C₁-C₆ alkylenyl)-S(O)₂R^{x}, —(C₁-C₆ alkylenyl)—S(O)₂N(R^{x})₂, —(C₁-C₆ alkylenyl)—C(O)R^{x}, —(C₁-C₆ alkylenyl)—C(O)OR^{x}, —(C₁-C₆ alkylenyl)—C(O)N(R^{x})₂, —(C₁-C₆ alkylenyl)-C(O)N(R^{x})S(O)₂R^{y}, —(C₁-C₆ alkylenyl)—N(R^{x})₂, —(C₁-C₆ alkylenyl)-N(R^{x})C(O)R^{y}, —(C₁-C₆ alkylenyl)—N(R^{x})S(O)₂R^{y}, —(C₁-C₆ alkylenyl)-N(R^{x})C(O)O(R³), —(C₁-C₆ alkylenyl)—N(R^{x})C(O)N(R^{x})₂, or —(C₁-C₆ alkylenyl)-N(R^{x})S(O)₂N(R^{x})₂;
R^{s} and R^{t}, at each occurrence, are each independently C₁-C₆ alkyl, C₂-C₆ alkenyl, C₂-C₆ alkynyl, halogen, C₁-C₆ haloalkyl, oxo, —CN,
   NO₂, —OR^{x}, —OC(O)R^{y}, —OC(O)N(R^{x})₂, —SR^{x}, —S(O)₂R^{x}, —S(O)₂N(R^{x})₂, —C(O)R^{x}, —C(O)OR^{x}, —C(O)N(R^{x})₂, —C(O)N(R^{x})S(O)₂R^{y}, —N(R^{x})₂, —N(R^{x})C(O)R^{y}, —N(R^{x})S(O)₂R^{y}, —N(R^{x})C(O)O(R³), —N(R^{x})C(O)N(R^{x})₂, —N(R^{x})S(O)₂N(R^{x})₂, —(C₁-C₆ alkylenyl)-CN, —(C₁-C₆ alkylenyl)—OR^{x}, —(C₁-C₆ alkylenyl)—OC(O)R^{y}, —(C₁-C₆ alkylenyl)—OC(O)N(R^{x})₂, —(C₁-C₆ alkylenyl)-SR^{x}, —(C₁-C₆ alkylenyl)-S(O)₂R^{x}, —(C₁-C₆ alkylenyl)—S(O)₂N(R^{x})₂, —(C₁-C₆ alkylenyl)—C(O)R^{x}, —(C₁-C₆ alkylenyl)—C(O)OR^{x}, —(C₁-C₆ alkylenyl)—C(O)N(R^{x})₂, —(C₁-C₆ alkylenyl)-C(O)N(R^{x})S(O)₂R^{y}, —(C₁-C₆ alkylenyl)—N(R^{x})₂, —(C₁-C₆ alkylenyl)-N(R^{x})C(O)R^{y}, —(C₁-C₆ alkylenyl)—N(R^{x})S(O)₂R^{y}, —(C₁-C₆ alkylenyl)-N(R^{x})C(O)O(R³), —(C₁-C₆ alkylenyl)—N(R^{x})C(O)N(R^{x})₂, or —(C₁-C₆ alkylenyl)-N(R^{x})S(O)₂N(R^{x})₂;
R⁵ is hydrogen, halogen, C₁-C₃ alkyl, C₁-C₃ haloalkyl, or —(C₁-C₃ alkylenyl)-OR^{x};
R⁶ is hydrogen, halogen, C₁-C₆ alkyl, or C₁-C₆ haloalkyl;
R⁷ is hydrogen, halogen, C₁-C₆ alkyl, C₁-C₆ haloalkyl, —(C₁-C₆ alkylenyl)-OR^{x}, —(C₁-C₆ alkylenyl)—S(O)₂R′, or —(C₁-C₆ alkylenyl)—S(O)₂N(R^{x})₂;
R^{x}, at each occurrence, is independently hydrogen, C₁-C₆ alkyl, or C₁-C₆ haloalkyl; and
R^{y}, at each occurrence, is independently C₁-C₆ alkyl or C₁-C₆ haloalkyl.

In another aspect, the present invention provides for methods for treating or preventing disorders that are ameliorated by inhibition of BET. Such methods comprise of administering to the subject a therapeutically effective amount of a compound of formula (I), (I-a), (I-b), (I-c), or (I-d), alone or in combination with a pharmaceutically acceptable carrier.

Some of the methods are directed to treating or preventing an inflammatory disease or cancer or AIDS.

In another aspect, the present invention relates to methods of treating cancer in a subject comprising administering a therapeutically effective amount of a compound of formula (I), (I-a), (I-b), (I-c), or (I-d), or a pharmaceutically acceptable salt thereof, to a subject in need thereof. In certain embodiments, the cancer is selected from the group consisting of: acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenström's macroglobulinemia, testicular tumors, uterine cancer and Wilms' tumor. In certain embodiments, the methods further comprise administering a therapeutically effective amount of at least one additional therapeutic agent. In certain embodiments, the additional therapeutic agent is selected from the group consisting of cytarabine, bortezomib, and 5-azacitidine.

In another aspect, the present invention relates to methods of treating a disease or condition in a subject comprising administering a therapeutically effective amount of a compound of formula (I), (I-a), (I-b), (I-c), or (I-d), or a pharmaceutically acceptable salt thereof, to a subject in need thereof, wherein said disease or condition is selected from the group consisting of: Addison's disease, acute gout, ankylosing spondylitis, asthma, atherosclerosis, Behcet's disease, bullous skin diseases, chronic obstructive pulmonary disease (COPD), Crohn's disease, dermatitis, eczema, giant cell arteritis, glomerulonephritis, hepatitis, hypophysitis, inflammatory bowel disease, Kawasaki disease, lupus nephritis, multiple sclerosis, myocarditis, myositis, nephritis, organ transplant rejection, osteoarthritis, pancreatitis, pericarditis, polyarteritis nodosa, pneumonitis, primary biliary cirrhosis, psoriasis, psoriatic arthritis, rheumatoid arthritis, scleritis, sclerosing cholangitis, sepsis, systemic lupus erythematosus, Takayasu's Arteritis, toxic shock, thyroiditis, type I diabetes, ulcerative colitis, uveitis, vitiligo, vasculitis, and Wegener's granulomatosis. In certain embodiments, the methods further comprise administering a therapeutically effective amount of at least one additional therapeutic agent.

In another aspect, the present invention relates to methods of treating a chronic kidney disease or condition in a subject comprising administering a therapeutically effective amount of a compound of formula (I), (I-a), (I-b), (I-c), or (I-d), or a pharmaceutically acceptable salt thereof, to a subject in need thereof, wherein said disease or condition is selected from the group consisting of: diabetic nephropathy, hypertensive nephropathy, HIV-associated nephropathy, glomerulonephritis, lupus nephritis, IgA nephropathy, focal segmental glomerulosclerosis, membranous glomerulonephritis, minimal change disease, polycystic kidney disease, and tubular interstitial nephritis. In certain embodiments, the methods further comprise administering a therapeutically effective amount of at least one additional therapeutic agent.

In another aspect, the present invention relates to methods of treating an acute kidney injury or disease or condition in a subject comprising administering a therapeutically effective amount of a compound of formula (I), (I-a), (I-b), (I-c), or (I-d), or a pharmaceutically acceptable salt thereof, to a subject in need thereof, wherein said acute kidney injury or disease or condition is selected from the group consisting of: ischemia-reperfusion induced kidney disease, cardiac and major surgery induced kidney disease, percutaneous coronary intervention induced kidney disease, radio-contrast agent induced kidney disease, sepsis induced kidney disease, pneumonia induced kidney disease, and drug toxicity induced kidney disease. In certain embodiments, the methods further comprise administering a therapeutically effective amount of at least one additional therapeutic agent.

In another aspect, the present invention relates to methods of treating AIDS in a subject comprising administering a therapeutically effective amount of a compound of formula (I), (I-a), (I-b), (I-c), or (I-d), or a pharmaceutically acceptable salt thereof, to a subject in need thereof. In certain embodiments, the methods further comprise administering a therapeutically effective amount of at least one additional therapeutic agent.

In another aspect, the present invention relates to methods of treating obesity, dyslipidemia, hypercholesterolemia, Alzheimer's disease, metabolic syndrome, hepatic steatosis, type II diabetes, insulin resistance, diabetic retinopathy or diabetic neuropathy in a subject comprising administering a therapeutically effective amount of a compound of formula (I), (I-a), (I-b), (I-c), or (I-d), or a pharmaceutically acceptable salt thereof, to a subject in need thereof. In certain embodiments, the methods further comprise administering a therapeutically effective amount of at least one additional therapeutic agent.

In another aspect, the present invention relates to methods of preventing conception by inhibiting spermatogenesis in a subject comprising administering a therapeutically effective amount of a compound of formula (I), (I-a), (I-b), (I-c), or (I-d), or a pharmaceutically acceptable salt thereof, to a subject in need thereof. In certain embodiments, the methods further comprise administering a therapeutically effective amount of at least one additional therapeutic agent.

A further aspect of the invention provides the use of a compound of formula (I), (I-a), (I-b), (I-c), or (I-d), alone or in combination with at least one additional therapeutic agent, in the manufacture of a medicament for treating or preventing conditions and disorders disclosed herein, with or without a pharmaceutically acceptable carrier.

Pharmaceutical compositions comprising a compound of formula (I), (I-a), (I-b), (I-c), or (I-d), or a pharmaceutically acceptable salt, alone or in combination with at lease one additional therapeutic agent, are also provided.

### DETAILED DESCRIPTION

Disclosed herein are compounds of formula (I)

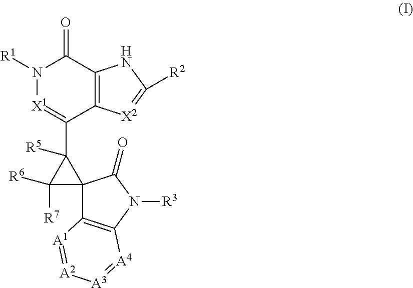

wherein R¹, R², R³, R⁵, R⁶, R⁷, A¹, A², A³, A⁴, X¹ and X² are defined above in the Summary of the Invention and below in the Detailed Description. Further, compositions comprising such compounds and methods for treating conditions and disorders using such compounds and compositions are also disclosed.

Compounds disclosed herein may contain one or more variable(s) that occur more than one time in any substituent or in the formulae herein. Definition of a variable on each occurrence is independent of its definition at another occurrence. Further, combinations of substituents are permissible only if such combinations result in stable compounds. Stable compounds are compounds, which can be isolated from a reaction mixture.

### a). Definitions

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds, reference to “a pharmaceutically acceptable carrier” means a single pharmaceutically acceptable carrier as well as one or more pharmaceutically acceptable carriers, and the like.

As used in the specification and the appended claims, unless specified to the contrary, the following terms have the meaning indicated:

The term “alkenyl” as used herein, means a straight or branched hydrocarbon chain containing from 2 to 10 carbons and containing at least one carbon-carbon double bond. The term “C₂-C₆ alkenyl” means an alkenyl group containing 2-6 carbon atoms. Non-limiting examples of C₂-C₆ alkenyl include buta-1,3-dienyl, ethenyl, 2-propenyl, 2-methyl-2-propenyl, 3-butenyl, 4-pentenyl, and 5-hexenyl.

The term “alkyl” as used herein, means a saturated, straight or branched hydrocarbon chain radical. In some instances, the number of carbon atoms in an alkyl moiety is indicated by the prefix “Cₓ-C_{y}”, wherein x is the minimum and y is the maximum number of carbon atoms in the substituent. Thus, for example, “C₁-C₆ alkyl” means an alkyl substituent containing from 1 to 6 carbon atoms, “C₁-C₄ alkyl” means an alkyl substituent containing from 1 to 4 carbon atoms, and “C₁-C₃ alkyl” means an alkyl substituent containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 1-methylbutyl, 2-methylbutyl, 3-methylbutyl, 3,3-dimethylbutyl, 1,1-dimethylpropyl, 1,2-dimethylpropyl, 2,2-dimethylpropyl, 1-methylpropyl, 2-methylpropyl, 1-ethylpropyl, and 1,2,2-trimethylpropyl. The terms “alkyl,” “C₁-C₆ alkyl,” “C₁-C₄ alkyl,” and “C₁-C₃ alkyl” used herein are unsubstituted, unless otherwise indicated.

The term “alkylene” or “alkylenyl” means a divalent radical derived from a straight or branched, saturated hydrocarbon chain, for example, of 1 to 10 carbon atoms or of 1 to 6 carbon atoms (C₁-C₆ alkylenyl) or of 1 to 4 carbon atoms or of 1 to 3 carbon atoms (C₁-C₃ alkylenyl) or of 2 to 6 carbon atoms (C₂-C₆ alkylenyl). Examples of alkylenyl include, but are not limited to, —CH₂—, —CH₂CH₂—, —C((CH₃)₂)—CH₂CH₂CH₂—, —C((CH₃)₂)—CH₂CH₂, —CH₂CH₂CH₂CH₂—, and —CH₂CH(CH₃)CH₂—.

The term “C₂-C₆ alkynyl” as used herein, means a straight or branched chain hydrocarbon radical containing from 2 to 6 carbon atoms and containing at least one carbon-carbon triple bond. Representative examples of C₂-C₆ alkynyl include, but are not limited, to acetylenyl, 1-propynyl, 2-propynyl, 3-butynyl, 2-pentynyl, and 1-butynyl.

The term “C₆-C₁₀ aryl” as used herein, means phenyl or a bicyclic aryl. The bicyclic aryl is naphthyl, or a phenyl fused to a C₃-C₆ monocyclic cycloalkyl, or a phenyl fused to a C₄-C₆ monocyclic cycloalkenyl. Non-limiting examples of the aryl groups include dihydroindenyl, indenyl, naphthyl, dihydronaphthalenyl, and tetrahydronaphthalenyl.

The term “C₃-C₇ monocyclic cycloalkyl” as used herein, means cyclopropyl, cyclobutyl, cyclopentyl, cyclohexyl, and cycloheptyl.

The term “C₃-C₆ monocyclic cycloalkyl” as used herein, means cyclopropyl, cyclobutyl, cyclopentyl, and cyclohexyl.

The term “C₄-C₆ monocyclic cycloalkenyl” as used herein, means cyclobutenyl, cyclopentenyl, and cyclohexenyl.

The term “halo” or “halogen” as used herein, means Cl, Br, I, and F.

The term “haloalkyl” as used herein, means an alkyl group, as defined herein, in which one, two, three, four, five, or six hydrogen atoms are replaced by halogen. The term “C₁-C₆ haloalkyl” means a C₁-C₆ alkyl group, as defined herein, in which one, two, three, four, five, or six hydrogen atoms are replaced by halogen. The term “C₁-C₄ haloalkyl” means a C₁-C₄ alkyl group, as defined herein, in which one, two, three, four, or five hydrogen atoms are replaced by halogen. The term “C₁-C₃ haloalkyl” means a C₁-C₃ alkyl group, as defined herein, in which one, two, three, four, or five hydrogen atoms are replaced by halogen. Representative examples of haloalkyl include, but are not limited to, chloromethyl, 2-fluoroethyl, 2,2-difluoroethyl, fluoromethyl, 2,2,2-trifluoroethyl, trifluoromethyl, difluoromethyl, pentafluoroethyl, 2-chloro-3-fluoropentyl, trifluorobutyl, and trifluoropropyl. The terms “haloalkyl,” “C₁-C₆ haloalkyl,” “C₁-C₄ haloalkyl,” and “C₁-C₃ haloalkyl,” as used herein are unsubstituted, unless otherwise indicated.

The term “5-11 membered heteroaryl” as used herein, means a monocyclic heteroaryl and a bicyclic heteroaryl. The monocyclic heteroaryl is a five- or six-membered hydrocarbon ring wherein at least one carbon ring atom is replaced by heteroatom independently selected from the group consisting of O, N, and S. The five-membered ring contains two double bonds. The five membered ring may have one heteroatom selected from O or S; or one, two, three, or four nitrogen atoms and optionally one oxygen or one sulfur atom. The six-membered ring contains three double bonds and one, two, three or four nitrogen atoms. Examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl. The bicyclic heteroaryl consists of a monocyclic heteroaryl fused to a phenyl, or a monocyclic heteroaryl fused to a monocyclic C₃-C₆ cycloalkyl, or a monocyclic heteroaryl fused to C₄-C₆ monocyclic cycloalkenyl, or a monocyclic heteroaryl fused to a monocyclic heteroaryl, or a monocyclic heteroaryl fused to a 4-7 membered monocyclic heterocycle. Representative examples of bicyclic heteroaryl groups include, but are not limited to, benzofuranyl, benzothienyl, benzoxazolyl, benzimidazolyl, benzoxadiazolyl, phthalazinyl, 2,6-dihydropyrrolo[3,4-c]pyrazol-5(4H)-yl, 6,7-dihydro-pyrazolo[1,5-a]pyrazin-5(4H)-yl, 6,7-dihydro-1,3-benzothiazolyl, imidazo[1,2-a]pyridinyl, indazolyl, indolyl, isoindolyl, isoquinolinyl, naphthyridinyl, pyridoimidazolyl, quinolinyl, 2,4,6,7-tetrahydro-5H-pyrazolo[4,3-c]pyridin-5-yl, thiazolo[5,4-b]pyridin-2-yl, thiazolo[5,4-d]pyrimidin-2-yl, and 5,6,7,8-tetrahydroquinolin-5-yl. The nitrogen atom in the heteroaryl rings may optionally be oxidized and may optionally be quaternized.

The term “4-11 membered heterocycle” as used herein, means a hydrocarbon ring radical of 4-11 carbon ring atoms wherein at least one carbon ring atom is replaced by atoms independently selected from the group consisting of O, N, and S. The 4-11 membered heterocycle ring may be a single ring (monocyclic) or have two or more rings (bicyclic or polycyclic). In certain embodiments, the monocyclic heterocycle is a four-, five-, six-, or seven-membered hydrocarbon ring wherein at least one carbon ring atom is replaced by atoms independently selected from the group consisting of O, N, and S. In certain embodiments, the monocyclic heterocycle is a 4-6 membered hydrocarbon ring wherein at least one carbon ring atom is replaced by heteroatom. A four-membered monocyclic heterocycle contains zero or one double bond, and one carbon ring atom replaced by an atom selected from the group consisting of O, N, and S. A five-membered monocyclic heterocycle contains zero or one double bond and one, two, or three carbon ring atoms replaced by atoms selected from the group consisting of O, N, and S. Examples of five-membered monocyclic heterocycles include those containing in the ring: 1 O; 1 S; 1 N; 2 N; 3 N; 1 S and 1 N; 1 S, and 2 N; 1 O and 1 N; or 1 O and 2 N. Non limiting examples of 5-membered monocyclic heterocyclic groups include 1,3-dioxolanyl, tetrahydrofuranyl, dihydrofuranyl, tetrahydrothienyl, dihydrothienyl, imidazolidinyl, oxazolidinyl, imidazolinyl, isoxazolidinyl, isothiazolidinyl, pyrazolidinyl, pyrazolinyl, pyrrolidinyl, 2-pyrrolinyl, 3-pyrrolinyl, thiazolinyl, and thiazolidinyl. A six-membered monocyclic heterocycle contains zero, one, or two double bonds and one, two, or three carbon ring atoms replaced by heteroatoms selected from the group consisting of O, N, and S. Examples of six-membered monocyclic heterocycles include those containing in the ring: 1 O; 2 O; 1 S; 2 S; 1 N; 2 N; 3 N; 1 S, 1 O, and 1 N; 1 S and 1 N; 1 S and 2 N; 1 S and 1 O; 1 S and 2 O; 1 O and 1 N; and 1 O and 2 N. Examples of six-membered monocyclic heterocycles include 1,3-oxazinanyl, tetrahydropyranyl, dihydropyranyl, 1,6-dihydropyridazinyl, 1,2-dihydropyrimidinyl, 1,6-dihydropyrimidinyl, dioxanyl, 1,4-dithianyl, hexahydropyrimidinyl, morpholinyl, piperazinyl, piperidinyl, 1,2,3,6-tetrahydropyridinyl, tetrahydrothiopyranyl, thiomorpholinyl, thioxanyl, and trithianyl. Seven- and eight-membered monocyclic heterocycles contains zero, one, two, or three double bonds and one, two, or three carbon ring atoms replaced by heteroatoms selected from the group consisting of O, N, and S. Examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, 1,6-dihydropyridazinyl, 1,2-dihydropyrimidinyl, 1,6-dihydropyrimidinyl, hexahydropyrimidinyl, imidazolinyl, imidazolidinyl, isoindolinyl, isothiazolinyl, isothiazolidinyl, isoxazolinyl, isoxazolidinyl, morpholinyl, oxadiazolinyl, oxadiazolidinyl, 1,3-oxazinanyl, oxazolinyl, 1,3-oxazolidinyl, oxetanyl, piperazinyl, piperidinyl, pyranyl, pyrazolinyl, pyrazolidinyl, pyrrolinyl, pyrrolidinyl, 1,2-dihydropyridinyl, tetrahydrofuranyl, tetrahydropyridinyl, tetrahydropyrimidinyl, tetrahydropyranyl, tetrahydrothienyl, thiadiazolinyl, thiadiazolidinyl, thiazolinyl, thiazolidinyl, thiomorpholinyl, thiopyranyl, and trithianyl. Polycyclic heterocycle groups contain two or more rings, and bicyclic heterocycles contain two rings. In certain embodiments, the polycyclic heterocycle groups contain 2 or 3 rings. The rings within the polycyclic and the bicyclic heterocycle groups are in a bridged, fused, or spiro orientation, or combinations thereof. In a spirocyclic heterocycle, one atom is common to two different rings. Non limiting examples of spirocyclic heterocycles include 4,6-diazaspiro[2.4]heptanyl, 6-azaspiro[3.4]octane, 2-oxa-6-azaspiro[3.4]octan-6-yl, and 2,7-diazaspiro[4.4]nonane. In a fused ring heterocycle, the rings share one common bond. Examples of fused bicyclic heterocycles are a 4-6 membered monocyclic heterocycle fused to a phenyl group, or a 4-6 membered monocyclic heterocycle fused to a C₃-C₆ monocyclic cycloalkyl, or a 4-6 membered monocyclic heterocycle fused to a C₄-C₆ monocyclic cycloalkenyl, or a 4-6 membered monocyclic heterocycle fused to a 4-6 membered monocyclic heterocycle. Examples of fused bicyclic heterocycles include, but are not limited to hexahydropyrano[3,4-b][1,4]oxazin-1(5H)-yl, hexahydropyrrolo[3,4-c]pyrrol-2(1H)-yl, hexahydro-1H-imidazo[5,1-c][1,4]oxazinyl, hexahydro-1H-pyrrolo[1,2-c]imidazolyl, hexahydrocyclopenta[c]pyrrol-3a(1H)-yl, and 3-azabicyclo[3.1.0]hexanyl. In a bridged heterocycle, the rings share at least two non-adjacent atoms. Examples of such bridged heterocycles include, but are not limited to, azabicyclo[2.2.1]heptyl (including 2-azabicyclo[2.2.1]hept-2-yl), 8-azabicyclo[3.2.1]oct-8-yl, octahydro-2,5-epoxypentalene, hexahydro-1H-1,4-methanocyclopenta[c]furan, aza-admantane (1-azatricyclo[3.3.1.1^{3,7}]decane), and oxa-adamantane (2-oxatricyclo[3.3.1.1^{3,7}]decane). The nitrogen and sulfur heteroatoms in the heterocycle rings may optionally be oxidized (e.g. 1,1-dioxidotetrahydrothienyl, 1,1-dioxido-1,2-thiazolidinyl, 1,1-dioxidothiomorpholinyl)) and the nitrogen atoms may optionally be quaternized.

The term “4-7 membered monocyclic heterocycle” as used herein, means a four-, five-, six-, or seven-membered monocyclic heterocycle, as defined herein above.

The phenyl, the C₆-C₁₀ aryls, the cycloalkyls, the cycloalkenyls, the heteroaryls, and the heterocycles, including the exemplary rings, are optionally substituted unless otherwise indicated; and are attached to the parent molecular moiety through any substitutable atom contained within the ring system.

The term “heteroatom” as used herein, means a nitrogen, oxygen, and sulfur.

The term “oxo” as used herein, means a ═O group.

The term “radiolabel” means a compound of the invention in which at least one of the atoms is a radioactive atom or a radioactive isotope, wherein the radioactive atom or isotope spontaneously emits gamma rays or energetic particles, for example alpha particles or beta particles, or positrons. Examples of such radioactive atoms include, but are not limited to, ³H (tritium), ¹⁴C, ¹¹C, ¹⁵O, ¹⁸F, ³⁵S, ¹²³I, and ¹²⁵I.

A moiety is described as “substituted” when a non-hydrogen radical is in the place of hydrogen radical of any substitutable atom of the moiety. Thus, for example, a substituted heterocycle moiety is a heterocycle moiety in which at least one non-hydrogen radical is in the place of a hydrogen radical on the heterocycle. It should be recognized that if there are more than one substitution on a moiety, each non-hydrogen radical may be identical or different (unless otherwise stated).

If a moiety is described as being “optionally substituted,” the moiety may be either (1) not substituted or (2) substituted. If a moiety is described as being optionally substituted with up to a particular number of non-hydrogen radicals, that moiety may be either (1) not substituted; or (2) substituted by up to that particular number of non-hydrogen radicals or by up to the maximum number of substitutable positions on the moiety, whichever is less. Thus, for example, if a moiety is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

The terms “treat”, “treating”, and “treatment” refer to a method of alleviating or abrogating a disease and/or its attendant symptoms. In certain embodiments, “treat,” “treating,” and “treatment” refer to ameliorating at least one physical parameter, which may not be discernible by the subject. In yet another embodiment, “treat”, “treating”, and “treatment” refer to modulating the disease or disorder, either physically (for example, stabilization of a discernible symptom), physiologically (for example, stabilization of a physical parameter), or both. In a further embodiment, “treat”, “treating”, and “treatment” refer to slowing the progression of the disease or disorder.

The terms “prevent”, “preventing”, and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring or developing a disease or disorder.

The phrase “therapeutically effective amount” means an amount of a compound, or a pharmaceutically acceptable salt thereof, sufficient to prevent the development of or to alleviate to some extent one or more of the symptoms of the condition or disorder being treated when administered alone or in conjunction with another therapeutic agent for treatment in a particular subject or subject population. The “therapeutically effective amount” may vary depending on the compound, the disease and its severity, and the age, weight, health, etc., of the subject to be treated. For example in a human or other mammal, a therapeutically effective amount may be determined experimentally in a laboratory or clinical setting, or may be the amount required by the guidelines of the United States Food and Drug Administration, or equivalent foreign agency, for the particular disease and subject being treated.

The term “subject” is defined herein to refer to animals such as mammals, including, but not limited to, primates (e.g., humans), cows, sheep, goats, pigs, horses, dogs, cats, rabbits, rats, mice and the like. In one embodiment, the subject is a human. The terms “human,” “patient,” and “subject” are used interchangeably herein.

The term ‘at least one additional therapeutic agent’ means one to four therapeutic agents other than the compounds of the invention. In one embodiment it means one to three additional therapeutic agents. In further embodiments it means one or two additional therapeutic agents. In a yet further embodiment it means one additional therapeutic agent.

b. Compounds

Compounds of the invention have the general formula (I) as described above.

Particular values of variable groups in compounds of formula (I) are as follows. Such values may be used where appropriate with any of the other values, definitions, claims or embodiments defined hereinbefore or hereinafter.

Compounds of the invention may exist as stereoisomers wherein asymmetric or chiral centers are present. In certain embodiments, the invention is directed to compounds of formula (I-a)

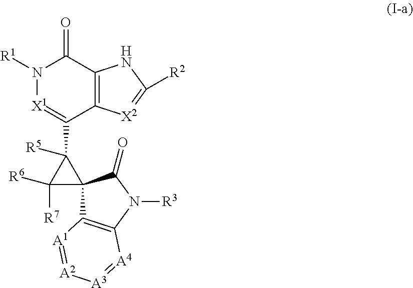

wherein R¹, R², R³, R⁵, R⁶, R⁷, X¹, X², A¹, A², A³, and A⁴ are as described in the Summary and the embodiments herein below.

In certain embodiments, the invention is directed to compounds of formula (I-b)

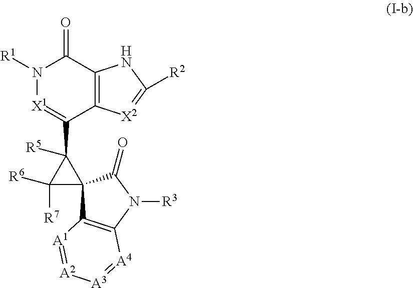

wherein R¹, R², R³, R⁵, R⁶, R⁷, X¹, X², A¹, A², A³, and A⁴ are as described in the Summary and the embodiments herein below.

In certain embodiments, the invention is directed to compounds of formula (I-c)

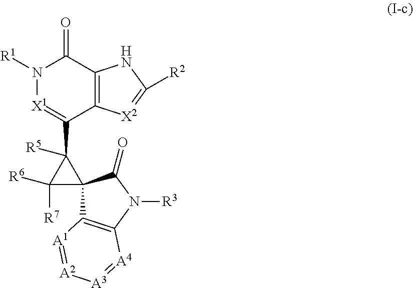

wherein R, R², R³, R⁵, R⁶, R⁷, X¹, X², A¹, A², A³, and A⁴ are as described in the Summary and the embodiments herein below.

In certain embodiments, the invention is directed to compounds of formula (I-d)

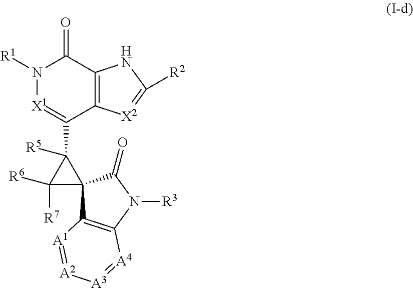

wherein R¹, R², R³, R⁵, R⁶, R⁷, X¹, X², A¹, A², A³, and A⁴ are as described in the Summary and the embodiments herein below.

In certain embodiments, R¹ is CH₃.

In certain embodiments, R² is hydrogen, G^{x1}, —C(O)OH, or —C(O)N(H)R^{A1}.

In certain embodiments, R² is G^{x1} or C(O)N(H)R^{A1}.

In certain embodiments, R² is G^{x1}. In some such embodiments, G^{x1} is 5-11 membered heteroaryl, C₃-C₇ monocyclic cycloalkyl, or 4-11 membered heterocycle. In some such embodiments, G^{x1} is a monocyclic heteroaryl or a C₃-C₇ monocyclic cycloalkyl. In some such embodiments, G^{x1} is 5-11 membered heteroaryl. In some such embodiments, G^{x1} is a monocyclic heteroaryl. In some such embodiments, G^{x1} is a bicyclic heteroaryl. In some such embodiments, G^{x1} is a C₃-C₇ monocyclic cycloalkyl.

In certain embodiments, R² is G^{x1} wherein G^{x1} is cyclopropyl, azetidinyl, pyrazolyl, 1,2-oxazolyl, imidazolyl, 1,3-oxazolyl, pyridinyl, pyrimidinyl, pyridazinyl, or benzimidazolyl.

In certain embodiments, R² is G^{x1} wherein G^{x1} is pyrazolyl.

In certain embodiments, R² is G^{x1} wherein G^{x1} is benzimidazolyl.

Each G^{x1}, including the exemplary rings, is optionally substituted with 1, 2, or 3 substituents independently selected from the group consisting of cyclopropyl and R^{s}; wherein the cyclopropyl is optionally substituted with 1, 2, or 3 substituents independently selected from the group consisting of halogen, C₁-C₆ haloalkyl, and C₁-C₆ alkyl.

In certain embodiments, each G^{x1}, including the exemplary rings, is optionally substituted with 1, 2, or 3 independently selected R^{s} groups.

In certain embodiments, R² is hydrogen or C(O)N(H)R^{A1}.

In certain embodiments, R² is hydrogen.

In certain embodiments, R² is —C(O)N(H)R^{A1}.

In certain embodiments, R² is —C(O)N(H)R^{A1} wherein R^{A1} is C₁-C₆ alkyl or G^{x2}. In some such embodiments, G^{x2} is phenyl or C₃-C₇ monocyclic cycloalkyl. In some such embodiments, G^{x2} is C₃-C₇ monocyclic cycloalkyl. In some such embodiments, G^{x2} is cyclobutyl or cyclopropyl. In some such embodiments, G^{x2} is cyclopropyl.

In certain embodiments, R^{A1} is G^{x2}, C₁-C₆ alkyl, or C₁-C₆ haloalkyl; wherein the C₁-C₆ haloalkyl and the C₁-C₆ alkyl are optionally substituted with 1 or 2 substituents independently selected from the group consisting of —OR^{B}, —CN, G^{x2}, and —N(R^{B})₂.

In certain embodiments, R^{A1} is G^{x2}, C₁-C₆ alkyl, or C₁-C₆ haloalkyl.

In certain embodiments, R^{A1} is C₁-C₆ alkyl or C₁-C₆ haloalkyl.

In certain embodiments, R^{A1} is C₁-C₆ alkyl.

In certain embodiments, R^{A1} is G^{x2}. In some such embodiments, G^{x2} is C₆-C₁₀ aryl or C₃-C₇ monocyclic cycloalkyl.

In certain embodiments, R^{A1} is G^{x2}; and G^{x2} is C₆-C₁₀ aryl. In some such embodiments, G^{x2} is phenyl.

In certain embodiments, R^{A1} is G^{x2}; and G^{x2} is 5-11 membered heteroaryl. In some such embodiments, G^{x2} is a monocyclic heteroaryl. In some such embodiments, G^{x2} is thiazolyl, oxazolyl, pyridinyl, pyrimidinyl, pyrazinyl, or pyridazinyl.

In certain embodiments, R^{A1} is G^{x2}; and G^{x2} is C₃-C₇ monocyclic cycloalkyl. In some such embodiments, G^{x2} is cyclopropyl or cyclobutyl. In some such embodiments, G^{x2} is cyclopropyl.

In certain embodiments, R^{A1} is G^{x2}; and G^{x2} is 4-11 membered heterocycle. In some such embodiments, G^{x2} is piperazinyl, piperidinyl, morpholinyl, 2H-1,3-benzodioxolyl or 2,3-dihydro-1,4-benzodioxinyl.

Each G^{x2}, including the exemplary rings, is optionally substituted with 1, 2, or 3 substituents independently selected from the group consisting of cyclopropyl and R^{s}.

In certain embodiments, each G^{x2}, including the exemplary rings, is optionally substituted with 1, 2, or 3 substituents independently selected R^{s} groups; wherein each R^{s} is independently C₁-C₆ alkyl, halogen, C₁-C₆ haloalkyl, —OR^{x}, or —(C₁-C₆ alkylenyl)-OR^{x}.

In certain embodiments, R² is —C(O)N(H)R^{A1} wherein R^{A1} is C₁-C₆ alkyl.

In certain embodiments, R² is —C(O)N(H)R^{A1} wherein R^{A1} is CH₂CH₃.

In certain embodiments, X¹ is N.

In certain embodiments, X¹ is C(R^{x1}).

In certain embodiments, R^{x1} is hydrogen.

In certain embodiments, X² is N.

In certain embodiments, X² is CH.

In certain embodiments, R³ is hydrogen, C₃-C₇ monocyclic cycloalkyl, C₁-C₆ alkyl, or C₁-C₆ haloalkyl, wherein the C₁-C₆ alkyl is optionally substituted with one substituent selected from the group consisting of —OR^{a} and phenyl; wherein the C₃-C₇ monocyclic cycloalkyl and the phenyl are each optionally substituted with 1, 2, or 3 independently selected R^{t} groups.

In certain embodiments, R³ is C₃-C₇ monocyclic cycloalkyl which is optionally substituted with 1, 2, or 3 independently selected R^{t} groups. In certain embodiments, R³ is cyclopropyl optionally substituted with 1, 2, or 3 independently selected R^{t} groups. In certain embodiments, R³ is unsubstituted cyclopropyl.

In certain embodiments, R³ is C₁-C₆ alkyl or C₁-C₆ haloalkyl. In certain embodiments, R³ is CH₃, CH₂CH₃, or CH₂CF₃.

In certain embodiments, R³ is C₁-C₆ alkyl. In certain embodiments, R³ is CH₃. In certain embodiments, R³ is CH₂CH₃.

In certain embodiments, A¹, A², A³, and A⁴ are CR⁴.

In certain embodiments, one of A¹, A², A³, and A⁴ is N, and the others are CR⁴.

In certain embodiments, two of A¹, A², A³, and A⁴ are N, and the others are CR⁴.

In certain embodiments, R⁴, at each occurrence, is independently hydrogen, C₁-C₆ alkyl, halogen, C₁-C₆ haloalkyl, —CN, NO₂, or —OR^{x}.

In certain embodiments, R⁵ is hydrogen, halogen, C₁-C₃ alkyl or C₁-C₃ haloalkyl.

In certain embodiments, R⁵ is hydrogen.

In certain embodiments, R⁵ is C₁-C₃ alkyl. In certain embodiments, R⁵ is CH₃.

In certain embodiments, R⁶ is hydrogen.

In certain embodiments, R⁷ is hydrogen.

Various embodiments of substituents R¹, R², R³, R⁵, R⁶, R⁷, X¹, X², A¹, A², A³, and A⁴ have been discussed above. These substituents embodiments can be combined to form various embodiments of compounds of formula (I), (I-a), (I-b), (I-c), and (I-d). All embodiments of compounds of formula (I), (I-a), (I-b), (I-c), and (I-d), formed by combining the substituent embodiments discussed above are within the scope of Applicant's invention, and some illustrative embodiments of the compounds of formula (I), (I-a), (I-b), (I-c), and (I-d) are provided below.

In one embodiment, the invention is directed to compounds of formula (I), (I-a), (I-b), (I-c), and (I-d), wherein
R¹ is CH₃;
X¹ is C(R^{x1}); and
X² is CH.

In one embodiment, the invention is directed to compounds of formula (I), (I-a), (I-b), (I-c), and (I-d), wherein
R¹ is CH₃;
A¹, A², A³, and A⁴ are CR⁴; and
each R⁴ is independently hydrogen, C₁-C₆ alkyl, halogen, C₁-C₆ haloalkyl, —CN, NO₂, or —OR^{x}.

In one embodiment, the invention is directed to compounds of formula (I), (I-a), (I-b), (I-c), and (I-d), wherein
R¹ is CH₃;
X¹ is C(R^{x1});
X² is CH;
A¹, A², A³, and A⁴ are CR⁴; and
each R⁴ is independently hydrogen, C₁-C₆ alkyl, halogen, C₁-C₆ haloalkyl, —CN, NO₂, or —OR^{x}.

In one embodiment, the invention is directed to compounds of formula (I), (I-a), (I-b), (I-c), and (I-d), wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x}; and
R² is hydrogen, G^{xi}, —C(O)OH, or —C(O)N(H)R^{A1}.

In one embodiment, the invention is directed to compounds of formula (I), (I-a), (I-b), (I-c), and (I-d), wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or OR^{x}; and
R² is G^{x1} wherein G^{x1} is monocyclic heteroaryl or C₃-C₇ monocyclic cycloalkyl; each of which is optionally substituted with 1, 2, or 3 independently selected R^{s} groups.

In one embodiment, the invention is directed to compounds of formula (I), (I-a), (I-b), (I-c), and (I-d), wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x}; and
R² is —C(O)N(H)R^{A1} wherein R^{A1} is C₁-C₆ alkyl or G^{x2}.

In one embodiment, the invention is directed to compounds of formula (I), (I-a), (I-b), (I-c), and (I-d), wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x}; and
R³ is hydrogen, C₃-C₇ monocyclic cycloalkyl, C₁-C₆ alkyl, or C₁-C₆ haloalkyl, wherein the C₁-C₆ alkyl is optionally substituted with one substituent selected from the group consisting of —OR^{a} and phenyl; wherein the C₃-C₇ monocyclic cycloalkyl and the phenyl are each optionally substituted with 1, 2, or 3 independently selected R^{t} groups.

In one embodiment, the invention is directed to compounds of formula (I), (I-a), (I-b), (I-c), and (I-d), wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x}; and
R³ is C₁-C₆ alkyl or C₁-C₆ haloalkyl.

In one embodiment, the invention is directed to compounds of formula (I), (I-a), (I-b), (I-c), and (I-d), wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x};
R² is G^{x1} or —C(O)N(H)R^{A1}; and
R³ is C₁-C₆ alkyl.

In one embodiment, the invention is directed to compounds of formula (I), (I-a), (I-b), (I-c), and (I-d), wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x};
R² is G^{x1} wherein G^{x1} is monocyclic heteroaryl which is optionally substituted with 1, 2, or 3 independently selected R^{s} groups; and
R³ is C₁-C₆ alkyl.

In one embodiment, the invention is directed to compounds of formula (I), (I-a), (I-b), (I-c), and (I-d), wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x};
R² is —C(O)N(H)R^{A1} wherein R^{A1} is G^{x2};
G^{x2} is C₃-C₇ monocyclic cycloalkyl optionally substituted with 1, 2, or 3 independently selected R^{s} groups; and
R³ is C₁-C₆ alkyl.

Compound of the invention are named by using Name 2016 naming algorithm by Advanced Chemical Development or Struct=Name naming algorithm as part of CHEMDRAW® ULTRA v. 15.0.0.106.

Compounds of the invention may exist as stereoisomers wherein asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45: 13-30. The invention contemplates various stereoisomers and mixtures thereof and these are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography and optional liberation of the optically pure product from the auxiliary as described in Furniss, Hannaford, Smith, and Tatchell, “Vogel's Textbook of Practical Organic Chemistry”, 5th edition (1989), Longman Scientific & Technical, Essex CM20 2JE, England, or (2) direct separation of the mixture of optical enantiomers on chiral chromatographic columns or (3) fractional recrystallization methods.

Certain names are prefixed by rac- (for racemic), denoting a racemic mixtures of two enantiomers in the ratio of about 1:1. For example, rac-N-ethyl-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide means about 1:1 mixture of N-ethyl-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide and N-ethyl-6-methyl-4-[(1S,2R)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide.

Compounds of the invention may exist as cis or trans isomers, wherein substituents on a ring may attached in such a manner that they are on the same side of the ring (cis) relative to each other, or on opposite sides of the ring relative to each other (trans). For example, cyclobutane may be present in the cis or trans configuration, and may be present as a single isomer or a mixture of the cis and trans isomers. Individual cis or trans isomers of compounds of the invention may be prepared synthetically from commercially available starting materials using selective organic transformations, or prepared in single isomeric form by purification of mixtures of the cis and trans isomers. Such methods are well-known to those of ordinary skill in the art, and may include separation of isomers by recrystallization or chromatography.

It should be understood that the compounds of the invention may possess tautomeric forms, as well as geometric isomers, and that these also constitute an aspect of the invention.

The present disclosure includes all pharmaceutically acceptable isotopically-labelled compounds of formula (I), (I-a), (I-b), (I-c), or (I-d) wherein one or more atoms are replaced by atoms having the same atomic number, but an atomic mass or mass number different from the atomic mass or mass number which predominates in nature. Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of formula (I), (I-a), (I-b), (I-c), or (I-d), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F , ¹⁵O and ¹³N, can be useful in Positron Emission Topography (PET) studies for examining substrate receptor occupancy. Isotopically-labeled compounds of formula (I), (I-a), (I-b), (I-c), or (I-d) may generally be prepared by conventional techniques known to those skilled in the art or by processes analogous to those described in the accompanying Examples using an appropriate isotopically-labeled reagents in place of the non-labeled reagent previously employed.

Thus, the formula drawings within this specification can represent only one of the possible tautomeric, geometric, or stereoisomeric forms. It is to be understood that the invention encompasses any tautomeric, geometric, or stereoisomeric form, and mixtures thereof, and is not to be limited merely to any one tautomeric, geometric, or stereoisomeric form utilized within the formula drawings.

Exemplary compounds of the present invention include, but are not limited to:

rac-N-ethyl-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide;

rac-N-cyclopropyl-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide;

N-cyclopropyl-6-methyl-4-[(1S,2R)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide;

N-cyclopropyl-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide;

rac-N-(3,3-difluorocyclobutyl)-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide;

6-methyl-4-[(1S,2R)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxylic acid;

6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxylic acid;

rac-6-methyl-4-[(1R,2S)-1′-methyl-2′-oxo-1′,2′-dihydrospiro[cyclopropane-1,3′-indol]-2-yl]-7-oxo-N-phenyl-6,7-dihydro-1H-pyrrolo[2,3-c]pyridine-2-carboxamide;

rac-N-(4-methoxyphenyl)-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide;

rac-N-[3-(hydroxymethyl)phenyl]-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide;

rac-N-cyclopropyl-4-[(1R,2S)-6′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;

rac-4-[(1R,2S)-6′-chloro-′-methyl-2′-oxo-1′,2′-dihydrospiro[cyclopropane-1,3′-indol]-2-yl]-N-cyclopropyl-6-methyl-7-oxo-6,7-dihydro-1H-pyrrolo[2,3-c]pyridine-2-carboxamide;

rac-N-cyclopropyl-4-[(1R,2S)-1′,6′-dimethyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;

rac-N-cyclopropyl-4-[(1R,2S)-6′-methoxy-′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;

rac-N-cyclopropyl-6-methyl-4-[(1R,2S)-1′-methyl-2′-oxo-6′-(trifluoromethoxy)-1′,2′-dihydrospiro[cyclopropane-1,3′-indol]-2-yl]-7-oxo-6,7-dihydro-1H-pyrrolo[2,3-c]pyridine-2-carboxamide;

rac-N-cyclopropyl-4-[(1R,2S)-5′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;

N-cyclopropyl-4-[(1S,2R)-5′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;

N-cyclopropyl-4-[(1R,2S)-5′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;

rac-N-cyclopropyl-6-methyl-7-oxo-4-[(1R,2S)-2′-oxo-1′,2′-dihydrospiro[cyclopropane-1,3′-indol]-2-yl]-6,7-dihydro-1H-pyrrolo[2,3-c]pyridine-2-carboxamide;

N-cyclopropyl-6-methyl-7-oxo-4-[(1S,2R)-2′-oxo-1′,2′-dihydrospiro[cyclopropane-1,3′-indol]-2-yl]-6,7-dihydro-1H-pyrrolo[2,3-c]pyridine-2-carboxamide;

N-cyclopropyl-6-methyl-7-oxo-4-[(1R,2S)-2′-oxo-1′,2′-dihydrospiro[cyclopropane-1,3′-indol]-2-yl]-6,7-dihydro-1H-pyrrolo[2,3-c]pyridine-2-carboxamide;

rac-N-cyclopropyl-4-[(1R,2S)-1′-ethyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;

rac-N-cyclopropyl-6-methyl-7-oxo-4-[(1R,2S)-2′-oxo-′-(2,2,2-trifluoroethyl)-1′,2′-dihydrospiro[cyclopropane-1,3′-indol]-2-yl]-6,7-dihydro-1H-pyrrolo[2,3-c]pyridine-2-carboxamide;

N-cyclopropyl-6-methyl-7-oxo-4-[(1S,2R)-2′-oxo-′-(2,2,2-trifluoroethyl)-1′,2′-dihydrospiro[cyclopropane-1,3′-indol]-2-yl]-6,7-dihydro-1H-pyrrolo[2,3-c]pyridine-2-carboxamide;

N-cyclopropyl-6-methyl-7-oxo-4-[(1R,2S)-2′oxo-1′-(2,2,2-trifluoroethyl)-1′,2′-dihydrospiro[cyclopropane-1,3′-indol]-2-yl]-6,7-dihydro-1H-pyrrolo[2,3-c]pyridine-2-carboxamide;

rac-N-cyclopropyl-4-[(1R,2S)-1′-cyclopropyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;

rac-N-cyclopropyl -4-[(1R,2S)-1′-(2-methoxyethyl)-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;

rac-4-{(1R,2S)-1′-[(4-aminophenyl)methyl]-2′-oxo-1′,2′-dihydrospiro[cyclopropane-1,3′-indol]-2-yl}-N-cyclopropyl-6-methyl-7-oxo-6,7-dihydro-1H-pyrrolo[2,3-c]pyridine-2-carboxamide;

rac-4-[(1R,2S)-7′-bromo-1′-methyl-2′-oxo-1′,2′-dihydrospiro[cyclopropane-1,3′-indol]-2-yl]-N-cyclopropyl-6-methyl-7-oxo-6,7-dihydro-1H-pyrrolo[2,3-c]pyridine-2-carboxamide;

rac-(1R,2S)-1′-methyl-2-(6-methyl-7-oxo-6,7-dihydro-1H-pyrrolo[2,3-c]pyridin-4-yl)spiro[cyclopropane-1,3′-indol]-2′(1H)-one;

rac-(1R,2S)-2-(2-cyclopropyl-6-methyl-7-oxo-6,7-dihydro-1H-pyrrolo[2,3-c]pyridin-4-yl)-1′-methylspiro[cyclopropane-1,3′-indol]-2′(1H)-one;

rac-tert-butyl 3-{6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridin-2-yl}azetidine-1-carboxylate;

rac-(1R,2S)-1′methyl-2-[6-methyl-2-(1-methyl-1H-pyrazol-4-yl)-7-oxo-6,7-dihydro-1H-pyrrolo[2,3-c]pyridin-4-yl]spiro[cyclopropane-1,3′-indol]-2′(1H)-one;

(1S,2R)-1′-methyl-2-[6-methyl-2-(1-methyl-1H-pyrazol-4-yl)-7-oxo-6,7-dihydro-1H-pyrrolo[2,3-c]pyridin-4-yl]spiro[cyclopropane-1,3′-indol]-2′(1H)-one;

(1R,2S)-1′-methyl-2-[6-methyl-2-(1-methyl-1H-pyrazol-4-yl)-7-oxo-6,7-dihydro-1H-pyrrolo[2,3-c]pyridin-4-yl]spiro[cyclopropane-1,3′-indol]-2′(1H)-one;

rac-(1R,2S)-2-[2-(1,3-dimethyl-1H-pyrazol-4-yl)-6-methyl-7-oxo-6,7-dihydro-1H-pyrrolo[2,3-c]pyridin-4-yl]-1′-methylspiro[cyclopropane-1,3′-indol]-2′(1H)-one;

rac-(1R,2S)-1′-methyl-2-[6-methyl-7-oxo-2-(1H-pyrazol-3-yl)-6,7-dihydro-1H-pyrrolo[2,3-c]pyridin-4-yl]spiro[cyclopropane-1,3′-indol]-2′(1H)-one;

rac-(1R,2R)-1′-methyl-2-[6-methyl-7-oxo-2-(1H-pyrazol-3-yl)-6,7-dihydro-1H-pyrrolo[2,3-c]pyridin-4-yl]spiro[cyclopropane-1,3′-indol]-2′(1H)-one;

rac-(1R,2S)-1′-methyl-2-[6-methyl-2-(3-methyl-1,2-oxazol-5-yl)-7-oxo-6,7-dihydro-1H-pyrrolo[2,3-c]pyridin-4-yl]spiro[cyclopropane-1,3′-indol]-2′(1H)-one;

rac-(1R,2R)-1′-methyl-2-[6-methyl-2-(3-methyl-1,2-oxazol-5-yl)-7-oxo-6,7-dihydro-1H-pyrrolo[2,3-c]pyridin-4-yl]spiro[cyclopropane-1,3′-indol]-2′(1H)-one;

rac-(1R,2S)-1′-methyl-2-[6-methyl-2-(1-methyl-1H-imidazol-2-yl)-7-oxo-6,7-dihydro-1H-pyrrolo[2,3-c]pyridin-4-yl]spiro[cyclopropane-1,3′-indol]-2′(1H)-one;

rac-(1R,2S)-1′-methyl-2-[6-methyl-2-(1,3-oxazol-2-yl)-7-oxo-6,7-dihydro-1H-pyrrolo[2,3-c]pyridin-4-yl]spiro[cyclopropane-1,3′-indol]-2′(1H)-one;

rac-(1R,2S)-1′-methyl-2-[6-methyl-2-(1-methyl-1H-imidazol-4-yl)-7-oxo-6,7-dihydro-1H-pyrrolo[2,3-c]pyridin-4-yl]spiro[cyclopropane-1,3′-indol]-2′(1H)-one;

rac-(1R,2S)-1′-methyl-2-[6-methyl-2-(1-methyl-1H-imidazol-5-yl)-7-oxo-6,7-dihydro-1H-pyrrolo[2,3-c]pyridin-4-yl]spiro[cyclopropane-1,3′-indol]-2′(1H)-one;

rac-(1R,2S)-1′-methyl-2-[6-methyl-7-oxo-2-(pyridin-2-yl)-6,7-dihydro-1H-pyrrolo[2,3-c]pyridin-4-yl]spiro[cyclopropane-1,3′-indol]-2′(1H)-one;

rac-(1R,2S)-1′-methyl-2-[6-methyl-2-(4-methylpyridin-2-yl)-7-oxo-6,7-dihydro-1H-pyrrolo[2,3-c]pyridin-4-yl]spiro[cyclopropane-1,3′-indol]-2′(1H)-one;

rac-(1R,2S)-1′-methyl-2-[6-methyl-7-oxo-2-(pyridin-3-yl)-6,7-dihydro-1H-pyrrolo[2,3-c]pyridin-4-yl]spiro[cyclopropane-1,3′-indol]-2′(1H)-one;

(1S,2R)-1′-methyl-2-[6-methyl-7-oxo-2-(pyridin-3-yl)-6,7-dihydro-1H-pyrrolo[2,3-c]pyridin-4-yl]spiro[cyclopropane-1,3′-indol]-2′(1H)-one;

(1R,2S)-1′-methyl-2-[6-methyl-7-oxo-2-(pyridin-3-yl)-6,7-dihydro-1H-pyrrolo[2,3-c]pyridin-4-yl]spiro[cyclopropane-1,3′-indol]-2′(1H)-one;

rac-(1R,2S)-1′-methyl-2-[6-methyl-7-oxo-2-(pyridin-4-yl)-6,7-dihydro-1H-pyrrolo[2,3-c]pyridin-4-yl]spiro[cyclopropane-1,3′-indol]-2′(1H)-one;

rac-(1R,2S)-1′-methyl-2-{6-methyl-7-oxo-2-[2-(trifluoromethyl)pyridin-4-yl]-6,7-dihydro-1H-pyrrolo[2,3-c]pyridin-4-yl}spiro[cyclopropane-1,3′-indol]-2′(1H)-one;

(1S,2R)-1′-methyl-2-{6-methyl-7-oxo-2-[2-(trifluoromethyl)pyridin-4-yl]-6,7-dihydro-1H-pyrrolo[2,3-c]pyridin-4-yl}spiro[cyclopropane-1,3′-indol]-2′(1H)-one;

(1R,2S)-1′-methyl-2-{6-methyl-7-oxo-2-[2-(trifluoromethyl)pyridin-4-yl]-6,7-dihydro-1H-pyrrolo[2,3-c]pyridin-4-yl}spiro[cyclopropane-1,3′-indol]-2′(1H)-one; rac-(1R,2S)-1′-methyl-2-[6-methyl-7-oxo-2-(pyrimidin-5-yl)-6,7-dihydro-1H-pyrrolo[2,3-c]pyridin-4-yl]spiro[cyclopropane-1,3′-indol]-2′(1H)-one;

rac-(1R,2S)-1′-methyl-2-[6-methyl-7-oxo-2-(pyrimidin-4-yl)-6,7-dihydro-1H-pyrrolo[2,3-c]pyridin-4-yl]spiro[cyclopropane-1,3′-indol]-2′(1H)-one;

rac-(1R,2S)-1′-methyl-2-[6-methyl-7-oxo-2-(pyridazin-4-yl)-6,7-dihydro-1H-pyrrolo[2,3-c]pyridin-4-yl]spiro[cyclopropane-1,3′-indol]-2′(1H)-one;

rac-1,3-dimethyl-5-{6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridin-2-yl}pyrimidine-2,4(1H,3H)-dione; and

rac-N-cyclopropyl-4-[(1R,2S)-1′,2-dimethyl-2′-oxo-6′-(trifluoromethoxy)-1′,2′-dihydrospiro[cyclopropane-1,3′-indol]-2-yl]-6-methyl-7-oxo-6,7-dihydro-1H-pyrrolo[2,3-c]pyridine-2-carboxamide.

Compounds of formula (I), (I-a), (I-b), (I-c), or (I-d) may be used in the form of pharmaceutically acceptable salts. The phrase “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio.

Pharmaceutically acceptable salts have been described in S. M. Berge et al. J. Pharmaceutical Sciences, 1977, 66: 1-19.

Compounds of formula (I), (I-a), (I-b), (I-c), or (I-d) may contain either a basic or an acidic functionality, or both, and may be converted to a pharmaceutically acceptable salt, when desired, by using a suitable acid or base. The salts may be prepared in situ during the final isolation and purification of the compounds of the invention.

Examples of acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isothionate), lactate, malate, maleate, methanesulfonate, nicotinate, 2-naphthalenesulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as, but not limited to, methyl, ethyl, propyl, and butyl chlorides, bromides and iodides; dialkyl sulfates like dimethyl, diethyl, dibutyl and diamyl sulfates; long chain halides such as, but not limited to, decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulfuric acid, and phosphoric acid and such organic acids as acetic acid, fumaric acid, maleic acid, 4-methylbenzenesulfonic acid, succinic acid and citric acid.

Basic addition salts may be prepared in situ during the final isolation and purification of compounds of this invention by reacting a carboxylic acid-containing moiety with a suitable base such as, but not limited to, the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as, but not limited to, lithium, sodium, potassium, calcium, magnesium and aluminum salts and the like and nontoxic quaternary ammonia and amine cations including ammonium, tetramethylammonium, tetraethylammonium, methylamine, dimethyl amine, trimethylamine, triethylamine, diethylamine, ethylamine and the like. Other examples of organic amines useful for the formation of base addition salts include ethylenediamine, ethanolamine, diethanolamine, piperidine, piperazine and the like.

The term “pharmaceutically acceptable prodrug” or “prodrug”as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use.

The present invention contemplates compounds of formula (I), (I-a), (I-b), (I-c), or (I-d) formed by synthetic means or formed by in vivo biotransformation of a prodrug.

Compounds described herein may exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

### General Synthesis

The compounds described herein, including compounds of general formula (I), (I-a), (I-b), (I-c), and (I-d) and specific examples, may be prepared, for example, through the reaction routes depicted in schemes 1-6. The variables A¹, A², A³, A⁴, X¹, X², R¹, R², R³, R⁴, R5, R⁶, R⁷, G^{x1}, R^{A1}, and R^{x1}, used in the following schemes have the meanings as set forth in the summary and detailed description sections unless otherwise noted.

Abbreviations used in the descriptions of the schemes and the specific examples have the following meanings: Cu(acac)₂ for copper acetylacetonate, DMSO for dimethyl sulfoxide, HATU for N-[(dimethylamino)-1H-1,2,3-triazolo-[4,5-b]pyridin-1-ylmethylene]-N-methylmethanaminium hexafluorophosphate N-oxide or 2-(7-azabenzotriazol-1-yl)-N,N,N′,N′-tetramethyluronium hexafluorophosphate or 1-[bis(dimethylamino)methylene]-1H-1,2,3-triazolo[4,5-b]pyridinium 3-oxid hexafluorophosphate, HPLC for high performance liquid chromatography, mL for milliliter, μL for mcroiter, LDA for lithium N,N-diisopropylamide, LC for liquid chromatography, MS for mass spectrometry, NMR for nuclear magnetic resonance, OAc for acetate, psi for pounds per square inch, Pd₂(dba)₃ for tris(dibenzylideneacetone)dipalladium(0), PdCl₂(dppf) for [1,1′-bis(diphenylphosphino)ferrocene]dichloropalladium(II), Pd(PPh₃)₄ for tetrakis(triphenylphosphine)palladium(0), Ra-Ni for Raney nickel, SFC for Supercritical Fluid Chromatography, Ts or tosyl for p-toluenesulfonyl, and X-phos for 2-dicyclohexylphosphino-2′,4′,6′-triisopropylbiphenyl.

Compounds of general formula (I) may be prepared by treating alkenyl compounds (3) with diazo compounds of general structure (4) as illustrated in Scheme 1. The reaction is typically catalyzed by a transition metal complex derived from, for example, rhodium, copper, ruthenium, palladium, cobalt, or iron. Examples include, but are not limited to, Rh₄(OAc)₂ and Cu(acac)₂ (H. M. L Davies, E. G. Antoulinakis Org. React. 2001, 57, 1-326; M. P. Doyle, D. C. Forbes Chem. Rev. 1998, 98, 911-936). The catalyst may include a ligand that is chiral or achiral, and the reaction may selectively produce single stereoisomers or mixtures of stereoisomers.

A compound of formula (3), in turn, may be prepared by treating an alkenyl halide or alkenyl triflate with a boronic acid or derivative thereof (e.g. boronic esters) under Suzuki coupling conditions (N. Miyaura and A. Suzuki, Chem. Rev. 1995, 95:2457-2483, J. Organomet. Chem. 1999, 576:147-148). Thus, coupling of compounds of formula (1) wherein R¹⁰¹ is Br, Cl, I, or triflate with compounds of formula (2) wherein R¹⁰³ is boronic acid or derivatives thereof (e.g. boronic esters) provides compounds of formula (3) wherein R¹⁰² is hydrogen or a suitable nitrogen protecting group (PG), R¹⁰⁴ and R¹⁰⁵ are hydrogen, halogen, alkyl, haloalkyl, or, —C(O)Oalkyl, and R¹⁰⁶ is R⁶. Examples of the nitrogen protecting group include, but are not limited to, p-tosyl, benzyl, and (trimethylsilyl)ethoxy)methyl. Alternatively, compounds of general formula (3) may be prepared from intermediates (1) wherein R¹⁰¹ is boronic acid or derivatives thereof (e.g. boronic esters) by coupling with compounds of formula (2) wherein R¹⁰³ is Br, Cl, I, or triflate. The (trimethylsilyl)ethoxy)methyl protecting group may be removed after coupling by treatment with tetra-n-butylammonium fluoride solution in a solvent such as, but not limited to, tetrahydrofuran.

Generally, the coupling reaction is effected in the presence of a palladium catalyst and a base, and optionally in the presence of a ligand, and in a suitable solvent at elevated temperature (for example, at about 80° C. to about 150° C.). The reaction may be facilitated by microwave irradiation. Examples of the palladium catalyst include, but are not limited to, tetrakis(triphenylphosphine)palladium(0), palladium(II)acetate, bis(triphenylphosphine)palladium(II)dichloride, and tris(dibenzylideneacetone) dipalladium(0). Examples of suitable bases that may be employed include, but are not limited to, acetates, carbonates, or phosphates of sodium, potassium, and cesium; and cesium fluoride. Examples of suitable ligands include, but not limited to, 1,3,5,7-tetramethyl-6-phenyl-2,4,8-trioxa-6-phosphaadamane, 2-dicyclohexylphosphino-2′,4′,6′-triisopropylbiphenyl (XPhos), and 1,1′-bis(diphenylphosphanyl) ferrocene. Non-limiting examples of suitable solvent include methanol, dimethoxyethane, N,N-dimethylformamide, dimethylsulfoxide, dioxane, tetrahydrofuran, and water, or a mixture thereof.

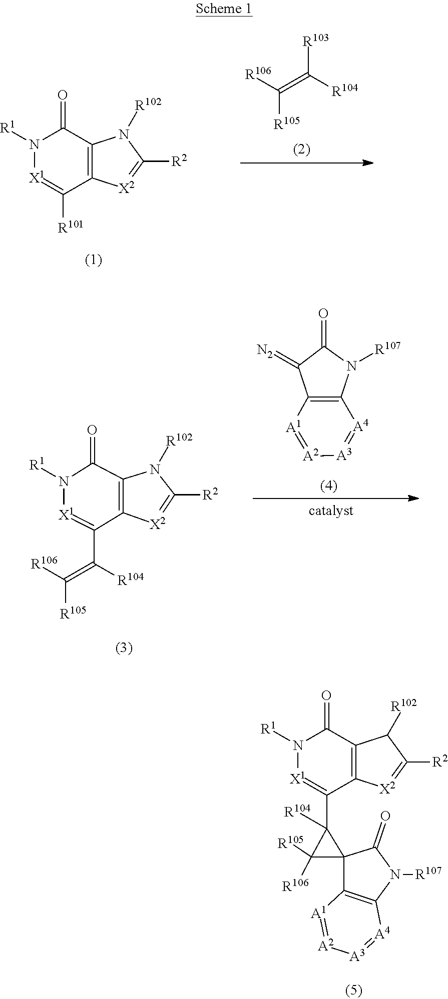

Intermediates (4) wherein R¹⁰⁷ is R³ or R¹⁰⁷ is a nitrogen protecting group and A¹, A², A³, and A⁴ are as defined, can be prepared from diones (6) by treatment with 4-methylbenzenesulfonohydrazide and a base such as NaOH (M. Ogeka, et al. Org. Lett. 2016, 18, 1358-1361) as illustrated in Scheme 2. Alternatively, (4) can be prepared from indolines or azaindolines (7) by for example treatment with a base such as potassium hydroxide and a diazo transfer reagent such as tosyl azide (A. Ford et al. Chem. Rev. 2015, 115, 9981-10080).

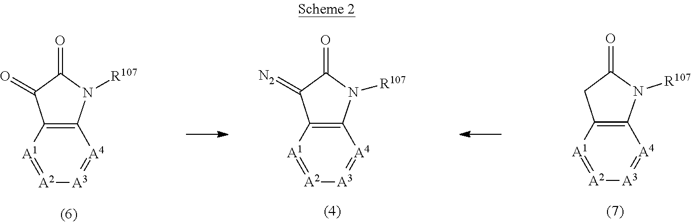

Compounds of formula (1) wherein X¹ is C(R^{x1}), X² is CH, R² is H or C(O)N(H)R^{A1}, R¹⁰⁰ is Br, Cl, I, or 1,3,2-dioxaborolane, and R¹⁰² is H or nitrogen protecting group, may be prepared using general synthetic route as shown in Scheme 3.

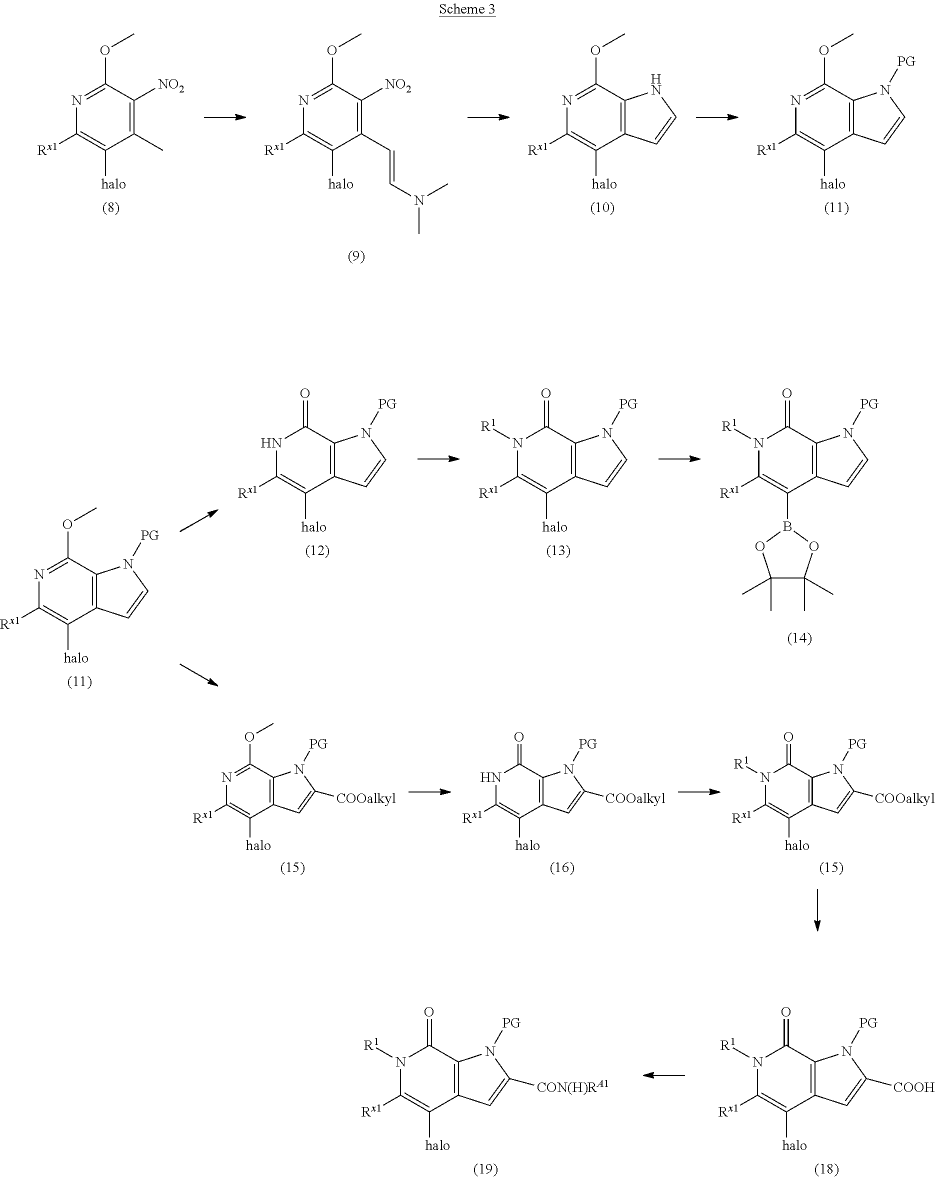

Treatment of compounds of formula (8) wherein halo is Br, Cl, or I, with 1,1-dimethoxy-N,N-dimethylmethanamine at elevated temperature (e.g. about 60° C. to about 100° C.), in the absence or presence of a base, and in a solvent such as, but not limited to, N,N-dimethylformamide, provide compounds of formula (9). Examples of bases that may be employed include, but not limited to, lithium or sodium methanolate. Catalytic hydrogenation of (9) in the presence of a catalyst such as, but not limited to, Raney-Nickel and under hydrogen atmosphere (about 30 psi) and in a solvent such as, but not limited to, ethyl acetate, at about room temperature generally affords compounds of formula (10). Protection of the nitrogen atom with protecting group (PG) such as, but not limited to, benzyl, tosyl, and (trimethylsilyl)ethoxy)methyl group may be derived from reaction with an appropriate halide, in the presence of a strong base such as, but not limited to, sodium hydride, to provide compounds of formula (11). Protection of the nitrogen atom may also be performed at different point of the synthetic route, for example, the protection may be conducted on intermediates (18) and/or (19).

Treatment of (11) with an acid such as, but not limited to, hydrochloric acid or hydrobromic acid and in a solvent such as, but not limited to, dioxane or water, at about 40° C. to about 100° C., typically provides compounds of formula (12).

Alkylation of (12) with an alkyl halide or mesylate, in the presence of a base such as, but not limited to, sodium hydride, cesium carbonate, or potassium carbonate, and in a solvent such as, but not limited to, N,N-dimethylformamide or dimethylsulfoxide at a temperature of about 0° C. to about 50° C. provides compounds of formula (13).

Treatment of the compounds of formula (13) with 4,4,4′,4′,5,5,5′,5′-octamethyl-2,2′-bi(1,3,2-dioxaborolane) generally affords compounds of formula (14). In general, the conversion may be facilitated by a palladium catalyst such as, but not limited to, tetrakis(triphenylphosphine)palladium(0), tris(dibenzylideneacetone)dipalladium(0), or palladium(II)acetate, an optional ligand such as, but not limited to, 2-dicyclohexylphosphino-2′,4′,6′-triisopropylbiphenyl, 2-dicyclohexylphosphino-2′,4′,6′-triisopropylbiphenyl (X-phos), or1,1′-bis(diphenylphosphanyl) ferrocene, and a base such as, but not limited to, carbonates, acetates, or phosphates of sodium, potassium, and cesium; and cesium fluoride. Non-limiting examples of suitable solvents include methanol, dimethoxyethane, N,N-dimethylformamide, dimethylsulfoxide, dioxane, tetrahydrofuran, and water, or a mixture thereof.

Conversation of (11) to (15) may be achieved by reaction with alkyl carbonochloridate in the presence of a base such as, but not limited to, lithium N,N-diisopropylamide. Esters of formula (17) may be obtained from (15) using the reaction conditions for the transformation of (11) to (13) outlined above. Hydrolysis of esters (17) provides acids of formula (18).

Acids of formula (18) may be transformed to the appropriate acid chloride by treatment with oxalyl chloride in the presence of catalytic amount of N,N-dimethylformamide at about room temperature, and in a suitable solvent such as, but not limited to, tetrahydrofuran or dichloromethane. The resulting acid chloride may be converted to amides of formula (19) by treatment with an amine of formula R^{A1}NH₂ in a solvent such as, but not limited to, tetrahydrofuran, N,N-dimethylformamide, or dichloromethane at a temperature from about room temperature to about 50° C., optionally in the presence of a base such as, but not limited to, triethylamine, diisopropylethylamine, or potassium carbonate, and optionally in the presence of a catalyst such as 4-dimethylaminopyridine.

Alternatively, acids of formula (18) may be reacted with the amine of formula R^{A1}NH₂ in a solvent such as, but not limited to, tetrahydrofuran or N,N-dimethylformamide in the presence of a coupling reagent such as 1,1′-carbonyldiimidazole (CDI), bis(2-oxo-3-oxazolidinyl)phosphinic chloride (BOPC1), 1,3-dicyclohexylcarbodiimide (DCC), polymer supported 1,3-dicyclohexylcarbodiimide (PS-DCC), O-(7-azabenzotriazol-1-yl)-N,N,N′,N′-tetramethyluronium hexafluorophosphate (HATU), or O-benzotriazol-1-yl-N,N,N′,N′-tetramethyluronium tetrafluoroborate (TBTU), in the presence or absence of a coupling auxiliary such as, but not limited to, 1-hydroxy-7-azabenzotriazole (HOAT) or 1-hydroxybenzotriazole hydrate (HOBT). The reaction may be generally conducted in the presence or absence of a base such as, but not limited to, N-methyl morpholine, triethylamine, or diisopropylethylamine.

Scheme 4 demonstrates a general approach to the alternative route to prepare compounds of formula (15).

Esters of formula (15) may be obtained from (a) treatment of (8) with dialkyl oxalate in the presence of a base such as, but not limited to, potassiumethoxide or sodiumethoxide, in a solvent such as, but not limited to, ethanol, dioxane, or diethyl ether, and at a temperature of about 40° C. to about 80° C.; (b) cyclization of the resulting (20) in the presence of iron and in ethanol and acetic acid, at a temperature of about 80° C. to about 100° C.; and (c) protection of the nitrogen atom of (21) using methodology known generally to one skilled in the art, for example, by treatment with tosyl halide in the presence of a base such as, but not limited to, sodium hydride.

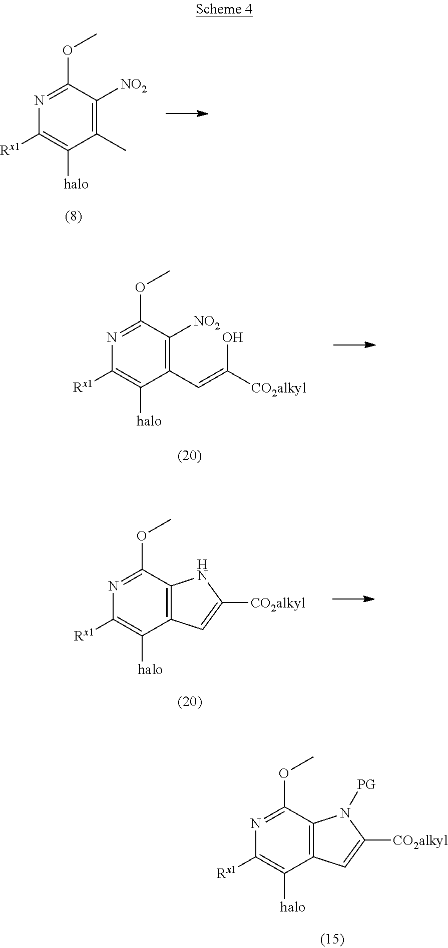

Compounds of formula (1) wherein X¹ is N, X² is CH, R² is H or C(O)N(H)R^{A1}, R¹⁰¹ is Br, Cl, I, or 1,3,2-dioxaborolane, and R¹⁰² is H or PG, may be prepared using general synthetic route as shown in Scheme 5.

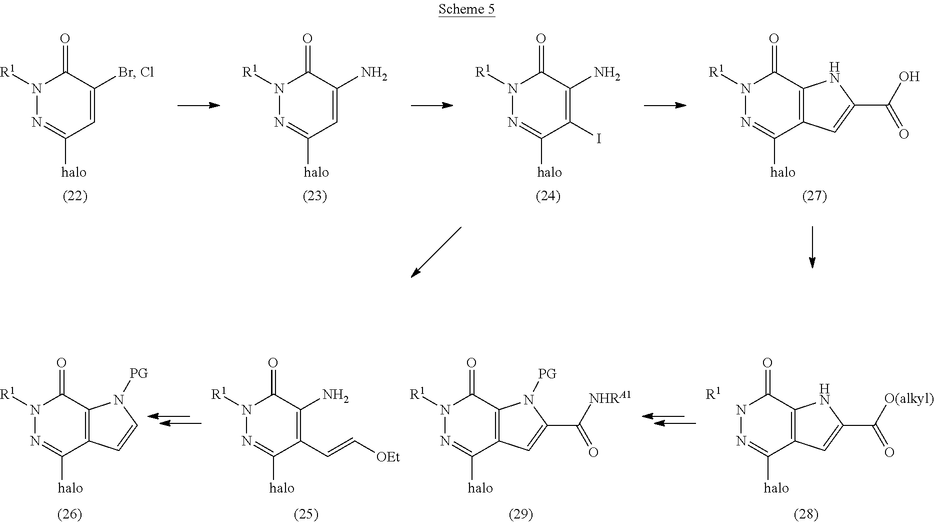

Treatment of (22) with ammonium hydroxide at about 100° C. to about 150° C. affords amines of formula (23).

Iodination of (23) with N-iodosuccinimide in a solvent such as, but not limited to, acetonitrile or acetone, at a temperature of about 40° C. to about 85° C., yields compounds of formula (24). Subsequent coupling with (E)-2-(2-ethoxyvinyl)-4,4,5,5-tetramethyl-1,3,2-dioxaborolane utilizing Suzuki coupling reaction conditions as described in Scheme 1 provides compounds of formula (25). Cyclization of (25) followed by protection of the nitrogen atom affords compounds of formula (26).

Cyclization of (25) may be accomplished in the presence of an acid such as, but not limited to, acetic acid or hydrochloric acid and at an elevated temperature (e.g. about 50° C. to about 100° C.).

Treatment of (24) with pyruvic acid in the presence of a palladium catalyst such as, but not limited to, palladium(II)acetate, and a base such as, but not limited to, 1,8-diazabicyclo[5.4.0]undec-7-ene, and in a solvent such as, but not limited to, N,N-dimethylformamide and at elevated temperature (e.g. at about 80° C. to about 150° C.) generally results in acids of formula (27). Esterification of (27) to (28) may be accomplished by reaction conditions known to one skilled in the art, for example, by treatment with an alcohol under acidic condition. Subsequent protection of (28) using reaction conditions described in Scheme 3 for the conversion of (10) to (11), followed by hydrolysis of the ester and conversion to the amides, provides compounds of formula (29).

Compounds of formula (1) wherein R¹⁰¹ is Br, Cl, or —C(R⁴)═C(R⁵)(R⁶), R¹⁰² is p-tosyl, and R² is G^{x1} may be prepared using synthetic route as outlined in Scheme 6.

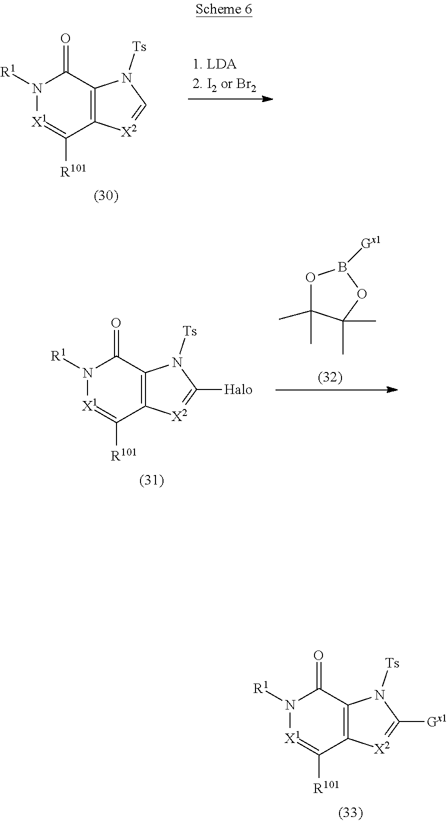

Iodination or bromination of compounds (30) with iodine or bromine in the presence of a base such as, but not limited to, lithium diisopropylamide provides compounds of formula (31). Compounds (31) may be treated with a boronic acid or esters of formula (32) using Suzuki coupling conditions as discussed in Scheme 1, to provide compounds of formula (33).

It can be appreciated that the synthetic schemes and specific examples as illustrated in the synthetic examples section are illustrative and are not to be read as limiting the scope of the invention as it is defined in the appended claims. All alternatives, modifications, and equivalents of the synthetic methods and specific examples are included within the scope of the claims.

Optimum reaction conditions and reaction times for each individual step can vary depending on the particular reactants employed and substituents present in the reactants used. Unless otherwise specified, solvents, temperatures and other reaction conditions can be readily selected by one of ordinary skill in the art. Specific procedures are provided in the Synthetic Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from commercially available materials using methods described in the chemical literature.

Routine experimentations, including appropriate manipulation of the reaction conditions, reagents and sequence of the synthetic route, protection of any chemical functionality that can not be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in T. Greene and P. Wuts, Protecting Groups in Organic Synthesis (3^{rd} ed.), John Wiley & Sons, NY (1999), which is incorporated herein by reference in its entirety. Synthesis of the compounds of the invention can be accomplished by methods analogous to those described in the synthetic schemes described hereinabove and in specific examples.

Starting materials, if not commercially available, can be prepared by procedures selected from standard organic chemical techniques, techniques that are analogous to the synthesis of known, structurally similar compounds, or techniques that are analogous to the above described schemes or the procedures described in the synthetic examples section.

When an optically active form of a compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be prepared by carrying out one of the above procedures using a pure geometric isomer as a starting material, or by resolution of a mixture of the geometric isomers of the compound or intermediates using a standard procedure such as chromatographic separation.

### Pharmaceutical Compositions

When employed as a pharmaceutical, a compound of the invention is typically administered in the form of a pharmaceutical composition. Such composition may be prepared in a manner well known in the pharmaceutical art and comprise a therapeutically effective amount of a compound of formula (I), (I-a), (I-b), (I-c), or (I-d), or a pharmaceutically acceptable salt thereof, alone or or in combination with at least one additional therapeutic agent, together with a pharmaceutically acceptable carrier. The phrase “pharmaceutical composition” refers to a composition suitable for administration in medical or veterinary use.

The pharmaceutical compositions that comprise a compound of formula (I), (I-a), (I-b), (I-c), or (I-d), alone or or in combination with at least one additional therapeutic agent, may be administered to the subjects orally, rectally, parenterally, intracisternally, intravaginally, intraperitoneally, topically (as by powders, ointments or drops), bucally or as an oral or nasal spray. The term “parenterally” as used herein, refers to modes of administration which include intravenous, intramuscular, intraperitoneal, intrasternal, subcutaneous, and intraarticular injection and infusion.

The term “pharmaceutically acceptable carrier” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such a propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents, which delay absorption such as aluminum monostearate and gelatin.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally-administered drug form may be accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In certain embodiments, solid dosage forms may contain from 1% to 95% (w/w) of a compound of formula I. In certain embodiments, the compound of formula I may be present in the solid dosage form in a range of from 5% to 70% (w/w). In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

The pharmaceutical composition may be a unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampules. Also, the unit dosage form can be a capsule, tablet, cachet, or lozenge itself, or it can be the appropriate number of any of these in packaged form. The quantity of active component in a unit dose preparation may be varied or adjusted from 0.1 mg to 1000 mg, from 1 mg to 100 mg, or from 1% to 95% (w/w) of a unit dose, according to the particular application and the potency of the active component. The composition can, if desired, also contain other compatible therapeutic agents.

The dose to be administered to a subject may be determined by the efficacy of the particular compound employed and the condition of the subject, as well as the body weight or surface area of the subject to be treated. The size of the dose also will be determined by the existence, nature, and extent of any adverse side-effects that accompany the administration of a particular compound in a particular subject. In determining the effective amount of the compound to be administered in the treatment or prophylaxis of the disorder being treated, the physician can evaluate factors such as the circulating plasma levels of the compound, compound toxicities, and/or the progression of the disease, etc. In general, the dose equivalent of a compound is from about 1 μg/kg to 100 mg/kg for a typical subject.

For administration, compounds of the formula (I), (I-a), (I-b), (I-c), or (I-d) may be administered at a rate determined by factors that can include, but are not limited to, the LD₅₀ of the compound, the pharmacokinetic profile of the compound, contraindicated drugs, and the side-effects of the compound at various concentrations, as applied to the mass and overall health of the subject. Administration can be accomplished via single or divided doses.

The compounds utilized in the pharmaceutical method of the invention may be administered at the initial dosage of about 0.001 mg/kg to about 100 mg/kg daily. In certain embodiments, the daily dose range is from about 0.1 mg/kg to about 10 mg/kg. The dosages, however, may be varied depending upon the requirements of the subject, the severity of the condition being treated, and the compound being employed. Determination of the proper dosage for a particular situation is within the skill of the practitioner and may be varied depending upon the requirements of the subject, the severity of the condition being treated, and the compound being employed. Treatment may be initiated with smaller dosages, which are less than the optimum dose of the compound. Thereafter, the dosage is increased by small increments until the optimum effect under circumstances is reached. For convenience, the total daily dosage may be divided and administered in portions during the day, if desired.

Solid compositions of a similar type may also be employed as fillers in soft and hard-filled gelatin capsules using such carriers as lactose or milk sugar as well as high molecular weight polyethylene glycols and the like.

The solid dosage forms of tablets, dragees, capsules, pills and granules may be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds may also be in micro-encapsulated form, if appropriate, with one or more of the above-mentioned carriers.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which may be prepared by mixing the compounds of this invention with suitable non-irritating carriers or carriers such as cocoa butter, polyethylene glycol or a suppository wax which are solid at room temperature but liquid at body temperature and therefore melt in the rectum or vaginal cavity and release the active compound.

Compounds of formula (I), (I-a), (I-b), (I-c), or (I-d) may also be administered in the form of liposomes. Liposomes generally may be derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form may contain, in addition to a compound of formula (I), (I-a), (I-b), (I-c), or (I-d), stabilizers, preservatives, excipients and the like. Examples of lipids include, but are not limited to, natural and synthetic phospholipids and phosphatidyl cholines (lecithins), used separately or together.

Methods to form liposomes have been described, see example, Prescott, Ed., Methods in Cell Biology, Volume XIV, Academic Press, New York, N.Y. (1976), p. 33 et seq.

Dosage forms for topical administration of a compound described herein include powders, sprays, ointments and inhalants. The active compound may be mixed under sterile conditions with a pharmaceutically acceptable carrier and any needed preservatives, buffers or propellants which may be required. Opthalmic formulations, eye ointments, powders and solutions are also contemplated as being within the scope of this invention.

### Methods of Use

The compounds of formula c, or pharmaceutically acceptable salts thereof, and pharmaceutical compositions comprising a compound of formula (I), (I-a), (I-b), (I-c), or (I-d), or a pharmaceutically acceptable salt thereof, may be administered to a subject suffering from a bromodomain-mediated disorder or condition. The term “administering” refers to the method of contacting a compound with a subject. Thus, the compounds of formula (I), (I-a), (I-b), (I-c), or (I-d) may be administered by injection, that is, intravenously, intramuscularly, intracutaneously, subcutaneously, intraduodenally, parentally, or intraperitoneally. Also, the compounds described herein may be administered by inhalation, for example, intranasally. Additionally, the compounds of formula (I), (I-a), (I-b), (I-c), or (I-d) may be administered transdermally, topically, via implantation, and transdermaly. In certain embodiments, the compounds of the formula (I), (I-a), (I-b), (I-c), or (I-d) may be delivered orally. The compounds may also be delivered rectally, bucally, intravaginally, ocularly, andially, or by insufflation. Bromodomain-mediated disorders and conditions may be treated prophylactically, acutely, and chronically using compounds of formula (I), (I-a), (I-b), (I-c), or (I-d), depending on the nature of the disorder or condition. Typically, the host or subject in each of these methods is human, although other mammals may also benefit from the administration of a compound of formula (I), (I-a), (I-b), (I-c), or (I-d).

A “bromodomain-mediated disorder or condition” is characterized by the participation of one or more bromodomains (e.g., BRD4) in the inception, manifestation of one or more symptoms or disease markers, severity, or progression of a disorder or condition. Accordingly, the invention provides a method for treating cancer, including, but not limited to acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenström's macroglobulinemia, testicular tumors, uterine cancer and Wilms' tumor. The method comprises the step of administering to a subject in need thereof a therapeutically effective amount of a compound of formula (I), (I-a), (I-b), (I-c), or (I-d), or a preferred embodiment thereof, with or without a pharmaceutically acceptable carrier.

The invention further provides a method for treating inflammatory diseases, inflammatory conditions, and autoimmune diseases, including, but not limited to: Addison's disease, acute gout, ankylosing spondylitis, asthma, atherosclerosis, Behcet's disease, bullous skin diseases, chronic obstructive pulmonary disease (COPD), Crohn's disease, dermatitis, eczema, giant cell arteritis, glomerulonephritis, hepatitis, hypophysitis, inflammatory bowel disease, Kawasaki disease, lupus nephritis, multiple sclerosis, myocarditis,myositis, nephritis, organ transplant rejection, osteoarthritis, pancreatitis, pericarditis, polyarteritis nodosa, pneumonitis, primary biliary cirrhosis, psoriasis, psoriatic arthritis, rheumatoid arthritis, scleritis, sclerosing cholangitis, sepsis, systemic lupus erythematosus, Takayasu's Arteritis, toxic shock, thyroiditis, type I diabetes, ulcerative colitis, uveitis, vitiligo, vasculitis, and Wegener's granulomatosis. The method comprises the step of administering to a subject in need thereof a therapeutically effective amount of a compound of formula (I), (I-a), (I-b), (I-c), or (I-d), or a preferred embodiment thereof, with or without a pharmaceutically acceptable carrier.

The invention further provides a method for treating diabetic nephropathy, hypertensive nephropathy, HIV-associated nephropathy, glomerulonephritis, lupus nephritis, IgA nephropathy, focal segmental glomerulosclerosis, membranous glomerulonephritis, minimal change disease, polycystic kidney disease, or tubular interstitial nephritis. The method comprises the step of administering to a subject in need thereof a therapeutically effective amount of a compound of formula (I), (I-a), (I-b), (I-c), or (I-d), or a preferred embodiment thereof, with or without a pharmaceutically acceptable carrier.

The invention further provides a method for treating acute kidney injury or disease or condition, wherein said acute kidney injury or disease or condition is selected from the group consisting of: ischemia-reperfusion induced kidney disease, cardiac and major surgery induced kidney disease, percutaneous coronary intervention induced kidney disease, radio-contrast agent induced kidney disease, sepsis induced kidney disease, pneumonia induced kidney disease, and drug toxicity induced kidney disease. The method comprises the step of administering to a subject in need thereof a therapeutically effective amount of a compound of formula (I), (I-a), (I-b), (I-c), or (I-d), or a preferred embodiment thereof, with or without a pharmaceutically acceptable carrier.

The invention further provides a method for treating chronic kidney disease or condition, wherein said disease or condition is selected from the group consisting of: diabetic nephropathy, hypertensive nephropathy, HIV-associated nephropathy, glomerulonephritis, lupus nephritis, IgA nephropathy, focal segmental glomerulosclerosis, membranous glomerulonephritis, minimal change disease, polycystic kidney disease, and tubular interstitial nephritis. The method comprises the step of administering to a subject in need thereof a therapeutically effective amount of a compound of formula (I), (I-a), (I-b), (I-c), or (I-d), or a preferred embodiment thereof, with or without a pharmaceutically acceptable carrier.

The invention further provides a method for treating AIDS. The method comprises the step of administering to a subject in need thereof a therapeutically effective amount of a compound of formula (I), (I-a), (I-b), (I-c), or (I-d), or a preferred embodiment thereof, with or without a pharmaceutically acceptable carrier.

In another embodiment, the present invention provides compounds of the invention, or pharmaceutical compositions comprising a compound of the invention, for use in medicine. In a particular embodiment, the present invention provides compounds of the invention, or pharmaceutical compositions comprising a compound of the invention, for use in the treatment of diseases or disorders as described herein above.

One embodiment is directed to the use of a compound according to formula (I), (I-a), (I-b), (I-c), or (I-d), or a pharmaceutically acceptable salt thereof in the preparation of a medicament. The medicament optionally can comprise at least one additional therapeutic agent. In some embodiments the medicament is for use in the treatment of diseases and disorders as described herein above.

This invention is also directed to the use of a compound according to formula (I), (I-a), (I-b), (I-c), or (I-d), or a pharmaceutically acceptable salt thereof in the manufacture of a medicament for the treatment of the diseases and disorders as described herein above. The medicament optionally can comprise at least one additional therapeutic agent.

The compounds of formula (I), (I-a), (I-b), (I-c), or (I-d) may be administered as the sole active agent or it may be co-administered with other therapeutic agents, including other compounds that demonstrate the same or a similar therapeutic activity and that are determined to be safe and efficacious for such combined administration. The term “co-administered” means the administration of two or more different therapeutic agents or treatments (e.g., radiation treatment) that are administered to a subject in a single pharmaceutical composition or in separate pharmaceutical compositions. Thus co-administration involves administration at the same time of a single pharmaceutical composition comprising two or more different therapeutic agents or administration of two or more different compositions to the same subject at the same or different times.

The compounds of the invention may be co-administered with a therapeutically effective amount of at least one additional therapeutic agent to treat cancer, where examples of the therapeutic agents include, such as radiation, alkylating agents, angiogenesis inhibitors, antibodies, antimetabolites, antimitotics, antiproliferatives, antivirals, aurora kinase inhibitors, apoptosis promoters (for example, Bcl-xL, Bcl-w and Bfl-1) inhibitors, activators of death receptor pathway, Bcr-Abl kinase inhibitors, BiTE (Bi-Specific T cell Engager) antibodies, antibody drug conjugates, biologic response modifiers, cyclin-dependent kinase inhibitors, cell cycle inhibitors, cyclooxygenase-2 inhibitors, DVDs (dual variable domain antibodies), leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of inhibitors of apoptosis proteins (IAPs), intercalating antibiotics, kinase inhibitors, kinesin inhibitors, Jak2 inhibitors, mammalian target of rapamycin inhibitors, microRNA's, mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, phosphoinositide-3 kinase (bromodomain) inhibitors, proteosome inhibitors, purine analogs, pyrimidine analogs, receptor tyrosine kinase inhibitors, etinoids/deltoids plant alkaloids, small inhibitory ribonucleic acids (siRNAs), topoisomerase inhibitors, ubiquitin ligase inhibitors, and the like, and in combination with at least one of these agents.

BiTE antibodies are bi-specific antibodies that direct T-cells to attack cancer cells by simultaneously binding the two cells. The T-cell then attacks the target cancer cell. Examples of BiTE antibodies include adecatumumab (Micromet MT201), blinatumomab (Micromet MT103) and the like. Without being limited by theory, one of the mechanisms by which T-cells elicit apoptosis of the target cancer cell is by exocytosis of cytolytic granule components, which include perforin and granzyme B. In this regard, Bcl-2 has been shown to attenuate the induction of apoptosis by both perforin and granzyme B. These data suggest that inhibition of Bcl-2 could enhance the cytotoxic effects elicited by T-cells when targeted to cancer cells (V. R. Sutton, D. L. Vaux and J. A. Trapani, J. of Immunology 1997, 158 (12), 5783).

SiRNAs are molecules having endogenous RNA bases or chemically modified nucleotides. The modifications do not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides, combinations thereof and the like. The siRNA can have varying lengths (e.g., 10-200 bps) and structures (e.g., hairpins, single/double strands, bulges, nicks/gaps, mismatches) and are processed in cells to provide active gene silencing. A double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

Multivalent binding proteins are binding proteins comprising two or more antigen binding sites. Multivalent binding proteins are engineered to have the three or more antigen binding sites and are generally not naturally occurring antibodies. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific (i.e., capable of binding one antigen) or multispecific (i.e., capable of binding two or more antigens). DVD binding proteins comprising two heavy chain DVD polypeptides and two light chain DVD polypeptides are referred to as DVD Ig's. Each half of a DVD Ig comprises a heavy chain DVD polypeptide, a light chain DVD polypeptide, and two antigen binding sites. Each binding site comprises a heavy chain variable domain and a light chain variable domain with a total of 6 CDRs involved in antigen binding per antigen binding site. Multispecific DVDs include DVD binding proteins that bind DLL4 and VEGF, or C-met and EFGR or ErbB3 and EGFR.

Alkylating agents include altretamine, AMD-473, AP-5280, apaziquone, bendamustine, brostallicin, busulfan, carboquone, carmustine (BCNU), chlorambucil, CLORETAZINE® (laromustine, VNP 40101M), cyclophosphamide, decarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, TREANDA® (bendamustine), treosulfan, rofosfamide and the like.

Angiogenesis inhibitors include endothelial-specific receptor tyrosine kinase (Tie-2) inhibitors, epidermal growth factor receptor (EGFR) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs, vascular endothelial growth factor receptor tyrosine kinase (VEGFR) inhibitors and the like.

Antimetabolites include ALIMTA® (pemetrexed disodium, LY231514, MTA), 5-azacitidine, XELODA® (capecitabine), carmofur, LEUSTAT® (cladribine), clofarabine, cytarabine, cytarabine ocfosfate, cytosine arabinoside, decitabine, deferoxamine, doxifluridine, eflornithine, EICAR (5-ethynyl-1-β-D-ribofuranosylimidazole-4-carboxamide), enocitabine, ethnylcytidine, fludarabine, 5-fluorouracil alone or in combination with leucovorin, GEMZAR® (gemcitabine), hydroxyurea, ALKERAN® (melphalan), mercaptopurine, 6-mercaptopurine riboside, methotrexate, mycophenolic acid, nelarabine, nolatrexed, ocfosfate, pelitrexol, pentostatin, raltitrexed, Ribavirin, triapine, trimetrexate, S-1, tiazofurin, tegafur, TS-1, vidarabine, UFT and the like.

Antivirals include ritonavir, hydroxychloroquine and the like.

Aurora kinase inhibitors include ABT-348, AZD-1152, MLN-8054, VX-680, Aurora A-specific kinase inhibitors, Aurora B-specific kinase inhibitors and pan-Aurora kinase inhibitors and the like.

Bcl-2 protein inhibitors include AT-101 ((-)gossypol), GENASENSE® (G3139 or oblimersen (Bcl-2-targeting antisense oligonucleotide)), IPI-194, IPI-565, N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide) (ABT-737), N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide (ABT-263), GX-070 (obatoclax) and the like.

Bcr-Abl kinase inhibitors include DASATINIB® (BMS-354825), GLEEVEC® (imatinib) and the like.

CDK inhibitors include AZD-5438, BMI-1040, BMS-032, BMS-387, CVT-2584, flavopyridol, GPC-286199, MCS-5A, PD0332991, PHA-690509, seliciclib (CYC-202, R-roscovitine), ZK-304709 and the like.

COX-2 inhibitors include ABT-963, ARCOXIA® (etoricoxib), BEXTRA® (valdecoxib), BMS347070, CELEBREX® (celecoxib), COX-189 (lumiracoxib), CT-3, DERAMAXX® (deracoxib), JTE-522, 4-methyl-2-(3,4-dimethylphenyl)-1-(4-sulfamoylphenyl-1H-pyrrole), MK-663 (etoricoxib), NS-398, parecoxib, RS-57067, SC-58125, SD-8381, SVT-2016, S-2474, T-614, VIOXX® (rofecoxib) and the like.

EGFR inhibitors include EGFR antibodies, ABX-EGF, anti-EGFR immunoliposomes, EGF-vaccine, EMD-7200, ERBITUX® (cetuximab), HR3, IgA antibodies, IRESSA® (gefitinib), TARCEVA® (erlotinib or OSI-774), TP-38, EGFR fusion protein, TYKERB® (lapatinib) and the like.

ErbB2 receptor inhibitors include CP-724-714, CI-1033 (canertinib), HERCEPTIN® (trastuzumab), TYKERB® (lapatinib), OMNITARG® (2C4, petuzumab), TAK-165, GW-572016 (ionafarnib), GW-282974, EKB-569, PI-166, dHER2 (HER2 vaccine), APC-8024 (HER-2 vaccine), anti-HER/2neu bispecific antibody, B7.her2IgG3, AS HER2 trifunctional bispecfic antibodies, mAB AR-209, mAB 2B-1 and the like.

Histone deacetylase inhibitors include depsipeptide, LAQ-824, MS-275, trapoxin, suberoylanilide hydroxamic acid (SAHA), TSA, valproic acid and the like.

HSP-90 inhibitors include 17-AAG-nab, 17-AAG, CNF-101, CNF-1010, CNF-2024, 17-DMAG, geldanamycin, IPI-504, KOS-953, MYCOGRAB® (human recombinant antibody to HSP-90), NCS-683664, PU24FC1, PU-3, radicicol, SNX-2112, STA-9090 VER49009 and the like.

Inhibitors of inhibitors of apoptosis proteins include HGS1029, GDC-0145, GDC-0152, LCL-161, LBW-242 and the like.

Antibody drug conjugates include anti-CD22-MC-MMAF, anti-CD22-MC-MMAE, anti-CD22-MCC-DM1, CR-011-vcMMAE, PSMA-ADC, MEDI-547, SGN-19Am SGN-35, SGN-75 and the like.

Activators of death receptor pathway include TRAIL, antibodies or other agents that target TRAIL or death receptors (e.g., DR4 and DR5) such as Apomab, conatumumab, ETR2-ST01, GDC0145, (lexatumumab), HGS-1029, LBY-135, PRO-1762 and trastuzumab.

Kinesin inhibitors include Eg5 inhibitors such as AZD4877, ARRY-520; CENPE inhibitors such as GSK923295A and the like.

JAK-2 inhibitors include CEP-701 (lesaurtinib), XL019 and INCB018424 and the like.

MEK inhibitors include ARRY-142886, ARRY-438162 PD-325901, PD-98059 and the like.

mTOR inhibitors include AP-23573, CCI-779, everolimus, RAD-001, rapamycin, temsirolimus, ATP-competitive TORC1/TORC2 inhibitors, including PI-103, PP242, PP30, Torin 1 and the like.

Non-steroidal anti-inflammatory drugs include AMIGESIC® (salsalate), DOLOBID® (diflunisal), MOTRIN® (ibuprofen), ORUDIS® (ketoprofen), RELAFEN® (nabumetone), FELDENE® (piroxicam), ibuprofen cream, ALEVE® (naproxen) and NAPROSYN® (naproxen), VOLTAREN® (diclofenac), INDOCIN® (indomethacin), CLINORIL® (sulindac), TOLECTIN® (tolmetin), LODINE® (etodolac), TORADOL® (ketorolac), DAYPRO® (oxaprozin) and the like.

PDGFR inhibitors include C-451, CP-673, CP-868596 and the like.

Platinum chemotherapeutics include cisplatin, ELOXATIN® (oxaliplatin) eptaplatin, lobaplatin, nedaplatin, PARAPLATIN® (carboplatin), satraplatin, picoplatin and the like.

Polo-like kinase inhibitors include BI-2536 and the like.

Phosphoinositide-3 kinase (PI3K) inhibitors include wortmannin, LY294002, XL-147, CAL-120, ONC-21, AEZS-127, ETP-45658, PX-866, GDC-0941, BGT226, BEZ235, XL765 and the like.

Thrombospondin analogs include ABT-510, ABT-567, ABT-898, TSP-1 and the like.

VEGFR inhibitors include AVASTIN® (bevacizumab), ABT-869, AEE-788, ANGIOZYME™ (a ribozyme that inhibits angiogenesis (Ribozyme Pharmaceuticals (Boulder, Colo.) and Chiron, (Emeryville, Calif.)), axitinib (AG-13736), AZD-2171, CP-547,632, IM-862, MACUGEN (pegaptamib), NEXAVAR® (sorafenib, BAY43-9006), pazopanib (GW-786034), vatalanib (PTK-787, ZK-222584), SUTENT® (sunitinib, SU-11248), VEGF trap, ZACTIMA™ (vandetanib, ZD-6474), GA101, ofatumumab, ABT-806 (mAb-806), ErbB3 specific antibodies, BSG2 specific antibodies, DLL4 specific antibodies and C-met specific antibodies, and the like.

Antibiotics include intercalating antibiotics aclarubicin, actinomycin D, amrubicin, annamycin, adriamycin, BLENOXANE® (bleomycin), daunorubicin, CAELYX® or MYOCET® (liposomal doxorubicin), elsamitrucin, epirbucin, glarbuicin, ZAVEDOS® (idarubicin), mitomycin C, nemorubicin, neocarzinostatin, peplomycin, pirarubicin, rebeccamycin, stimalamer, streptozocin, VALSTAR® (valrubicin), zinostatin and the like.

Topoisomerase inhibitors include aclarubicin, 9-aminocamptothecin, amonafide, amsacrine, becatecarin, belotecan, BN-80915, CAMPTOSAR® (irinotecan hydrochloride), camptothecin, CARDIOXANE® (dexrazoxine), diflomotecan, edotecarin, ELLENCE® or PHARMORUBICIN® (epirubicin), etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

Antibodies include AVASTIN® (bevacizumab), CD40-specific antibodies, chTNT-1/B, denosumab, ERBITUX® (cetuximab), HUMAX-CD4® (zanolimumab), IGF1R-specific antibodies, lintuzumab, PANOREX® (edrecolomab), RENCAREX® (WX G250), RITUXAN® (rituximab), ticilimumab, trastuzimab, CD20 antibodies types I and II and the like.

Hormonal therapies include ARIMIDEX® (anastrozole), AROMASIN® (exemestane), arzoxifene, CASODEX® (bicalutamide), CETROTIDE® (cetrorelix), degarelix, deslorelin, DESOPAN® (trilostane), dexamethasone, DROGENIL® (flutamide), EVISTA® (raloxifene), AFEMA™ (fadrozole), FARESTON® (toremifene), FASLODEX® (fulvestrant), FEMARA® (letrozole), formestane, glucocorticoids, HECTOROL® (doxercalciferol), RENAGEL® (sevelamer carbonate), lasofoxifene, leuprolide acetate, MEGACE® (megesterol), MIFEPREX® (mifepristone), NILANDRON™ (nilutamide), NOLVADEX® (tamoxifen citrate), PLENAXIS™ (abarelix), prednisone, PROPECTA® (finasteride), rilostane, SUPREFACT® (buserelin), TRELSTAR® (luteinizing hormone releasing hormone (LHRH)), VANTAS® (Histrelin implant), VETORYL® (trilostane or modrastane), ZOLADEX® (fosrelin, goserelin) and the like.

Deltoids and retinoids include seocalcitol (EB1089, CB1093), lexacalcitrol (KH1060), fenretinide, PANRETIN® (aliretinoin), ATRAGEN® (liposomal tretinoin), TARGRETIN® (bexarotene), LGD-1550 and the like.

PARP inhibitors include ABT-888 (veliparib), olaparib, KU-59436, AZD-2281, AG-014699, BSI-201, BGP-15, INO-1001, ONO-2231 and the like.

Plant alkaloids include, but are not limited to, vincristine, vinblastine, vindesine, vinorelbine and the like.

Proteasome inhibitors include VELCADE® (bortezomib), MG132, NPI-0052, PR-171 and the like.

Examples of immunologicals include interferons and other immune-enhancing agents. Interferons include interferon alpha, interferon alpha-2a, interferon alpha-2b, interferon beta, interferon gamma-1a, ACTIMMUNE® (interferon gamma-1b) or interferon gamma-n1, combinations thereof and the like. Other agents include ALFAFERONE®, (IFN-α), BAM-002 (oxidized glutathione), BEROMUN® (tasonermin), BEXXAR® (tositumomab), CAMPATH® (alemtuzumab), CTLA4 (cytotoxic lymphocyte antigen 4), decarbazine, denileukin, epratuzumab, GRANOCYTE® (lenograstim), lentinan, leukocyte alpha interferon, imiquimod, MDX-010 (anti-CTLA-4), melanoma vaccine, mitumomab, molgramostim, MYLOTARG™ (gemtuzumab ozogamicin), NEUPOGEN® (filgrastim), OncoVAC-CL, OVAREX® (oregovomab), pemtumomab (Y-muHMFG1), PROVENGE® (sipuleucel-T), sargaramostim, sizofilan, teceleukin, THERACYS® (Bacillus Calmette-Guerin), ubenimex, VIRULIZIN® (immunotherapeutic, Lorus Pharmaceuticals), Z-100 (Specific Substance of Maruyama (SSM)), WF-10 (Tetrachlorodecaoxide (TCDO)), PROLEUKIN® (aldesleukin), ZADAXIN® (thymalfasin), ZENAPAX® (daclizumab), ZEVALIN® (90Y-Ibritumomab tiuxetan) and the like.

Biological response modifiers are agents that modify defense mechanisms of living organisms or biological responses, such as survival, growth or differentiation of tissue cells to direct them to have anti-tumor activity and include krestin, lentinan, sizofiran, picibanil PF-3512676 (CpG-8954), ubenimex and the like.

Pyrimidine analogs include cytarabine (ara C or Arabinoside C), cytosine arabinoside, doxifluridine, FLUDARA® (fludarabine), 5-FU (5-fluorouracil), floxuridine, GEMZAR® (gemcitabine), TOMUDEX® (ratitrexed), TROXATYL™ (triacetyluridine troxacitabine) and the like.

Purine analogs include LANVIS® (thioguanine) and PURI-NETHOL® (mercaptopurine).

Antimitotic agents include batabulin, epothilone D (KOS-862), N-(2-((4-hydroxyphenyl)amino)pyridin-3-yl)-4-methoxybenzenesulfonamide, ixabepilone (BMS 247550), paclitaxel, TAXOTERE® (docetaxel), PNU100940 (109881), patupilone, XRP-9881 (larotaxel), vinflunine, ZK-EPO (synthetic epothilone) and the like.

Ubiquitin ligase inhibitors include MDM2 inhibitors, such as nutlins, NEDD8 inhibitors such as MLN4924 and the like.

Compounds of this invention may also be used as radiosensitizers that enhance the efficacy of radiotherapy. Examples of radiotherapy include external beam radiotherapy, teletherapy, brachytherapy and sealed, unsealed source radiotherapy and the like.

Additionally, compounds of formula (I), (I-a), (I-b), (I-c), or (I-d) may be combined with other chemotherapeutic agents such as ABRAXANE™ (ABI-007), ABT-100 (farnesyl transferase inhibitor), ADVEXIN® (Ad5CMV-p53 vaccine), ALTOCOR® or MIEVACOR® (lovastatin), AMPLIGEN® (poly I:poly C12U, a synthetic RNA), APTOSYN® (exisulind), AREDIA® (pamidronic acid), arglabin, L-asparaginase, atamestane (1-methyl-3,17-dione-androsta-1,4-diene), AVAGE® (tazarotene), AVE-8062 (combreastatin derivative) BEC2 (mitumomab), cachectin or cachexin (tumor necrosis factor), canvaxin (vaccine), CEAVAC® (cancer vaccine), CELEUK® (celmoleukin), CEPLENE® (histamine dihydrochloride), CERVARIX® (human papillomavirus vaccine), CHOP® (C: CYTOXAN® (cyclophosphamide); H: ADRIAMYCIN® (hydroxydoxorubicin); 0: Vincristine) (ONCOVIN® ; P: prednisone), CYPAT™ (cyproterone acetate), combrestatin A4P, DAB(389)EGF (catalytic and translocation domains of diphtheria toxin fused via a His-Ala linker to human epidermal growth factor) or TransMID-107R™ (diphtheria toxins), dacarbazine, dactinomycin, 5,6-dimethylxanthenone-4-acetic acid (DMXAA), eniluracil, EVIZON™ (squalamine lactate), DIMERICIINE® (T4N5 liposome lotion), discodermolide, DX-8951f (exatecan mesylate), enzastaurin, EP0906 (epithilone B), GARDASIL® (quadrivalent human papillomavirus (Types 6, 11, 16, 18) recombinant vaccine), GASTRIMIMUNE®, GENASENSE®, GMK (ganglioside conjugate vaccine), GVAX® (prostate cancer vaccine), halofuginone, histerelin, hydroxycarbamide, ibandronic acid, IGN-101, IL-13-PE38, IL-13-PE38QQR (cintredekin besudotox), IL-13-pseudomonas exotoxin, interferon-α, interferon-γ, JUNOVAN™ or MEPACT™ (mifamurtide), lonafarnib, 5,10-methylenetetrahydrofolate, miltefosine (hexadecylphosphocholine), NEOVASTAT® (AE-941), NEUTREXIN® (trimetrexate glucuronate), NIPENT® (pentostatin), ONCONASE® (a ribonuclease enzyme), ONCOPHAGE® (melanoma vaccine treatment), ONCOVAX® (IL-2 Vaccine), ORATHECIN™ (rubitecan), OSIDEM® (antibody-based cell drug), OVAREX® MAb (murine monoclonal antibody), paclitaxel, PANDIMEX™ (aglycone saponins from ginseng comprising 20(S)protopanaxadiol (aPPD) and 20(S)protopanaxatriol (aPPT)), panitumumab, PANVAC® -VF (investigational cancer vaccine), pegaspargase, PEG Interferon A, phenoxodiol, procarbazine, rebimastat, REMOVAB® (catumaxomab), REVLIMID® (lenalidomide), RSR13 (efaproxiral), SOMATULINE® LA (lanreotide), SORIATANE® (acitretin), staurosporine (Streptomyces staurospores), talabostat (PT100), TARGRETIN® (bexarotene), TAXOPREXIN® (DHA-paclitaxel), TELCYTA® (canfosfamide, TLK286), temilifene, TEMODAR® (temozolomide), tesmilifene, thalidomide, THERATOPE® (STn-KLH), thymitaq (2-amino-3,4-dihydro-6-methyl-4-oxo-5-(4-pyridylthio)quinazoline dihydrochloride), TNFERADE™ (adenovector: DNA carrier containing the gene for tumor necrosis factor-α), TRACLEER® or ZAVESCA® (bosentan), tretinoin (Retin-A), tetrandrine, TRISENOX® (arsenic trioxide), VIRULIZIN®, ukrain (derivative of alkaloids from the greater celandine plant), vitaxin (anti-alphavbeta3 antibody), XCYTRIN® (motexafin gadolinium), XINLAY™ (atrasentan), XYOTAX™ (paclitaxel poliglumex), YONDELIS® (trabectedin), ZD-6126, ZINECARD® (dexrazoxane), ZOMETA® (zolendronic acid), zorubicin and the like.

The compounds of the invention may also be co-administered with a therapeutically effective amount of at least one additional therapeutic agents to treat an inflammatory disease or condition, or autoimmune disease, where examples of the agents include, such as methotrexate, 6-mercaptopurine, azathioprine sulphasalazine, mesalazine, olsalazine chloroquinine/hydroxychloroquine, pencillamine, aurothiomalate (intramuscular and oral), azathioprine, cochicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g., NIK, IKK, p38 or MAP kinase inhibitors), IL-1β converting enzyme inhibitors, T-cell signalling inhibitors such as kinase inhibitors, metalloproteinase inhibitors, sulfasalazine, 6-mercaptopurines, angiotensin converting enzyme inhibitors, soluble cytokine receptors and derivatives thereof (e.g. soluble p55 or p75 TNF receptors and the derivatives p75TNFRIgG (etanercept) and p55TNFRIgG (Lenercept), sIL-1RI, sIL-1RII, sIL-6R), antiinflammatory cytokines (e.g. IL-4, IL-10, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline HCl, sulfadiazine, oxycodone HCl/acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL15, BIRB-796, SCIO-469, VX-702, AMG-548, VX-740, Roflumilast, IC-485, CDC-801, S1P1 agonists (such as FTY720), PKC family inhibitors (such as Ruboxistaurin or AEB-071) and Mesopram. In certain embodiments, combinations include methotrexate or leflunomide and in moderate or severe rheumatoid arthritis cases, cyclosporine and anti-TNF antibodies as noted above.

Non-limiting examples of therapeutic agents for inflammatory bowel disease with which a compound of formula (I), (I-a), (I-b), (I-c), or (I-d) may be co-administered include the following: budenoside; epidermal growth factor; corticosteroids; cyclosporin, sulfasalazine; aminosalicylates; 6-mercaptopurine; azathioprine; metronidazole; lipoxygenase inhibitors; mesalamine; olsalazine; balsalazide; antioxidants; thromboxane inhibitors; IL-1 receptor antagonists; anti-IL-1β monoclonal antibodies; anti-IL-6 monoclonal antibodies; growth factors; elastase inhibitors; pyridinyl-imidazole compounds; antibodies to or antagonists of other human cytokines or growth factors, for example, TNF, LT, IL-1, IL-2, IL-6, IL-7, IL-8, IL-12, IL-15, IL-16, IL-23, EMAP-II, GM-CSF, FGF, and PDGF; cell surface molecules such as CD2, CD3, CD4, CD8, CD25, CD28, CD30, CD40, CD45, CD69, CD90 or their ligands; methotrexate; cyclosporine; FK506; rapamycin; mycophenolate mofetil; leflunomide; NSAIDs, for example, ibuprofen; corticosteroids such as prednisolone; phosphodiesterase inhibitors; adenosine agonists; antithrombotic agents; complement inhibitors; adrenergic agents; agents which interfere with signalling by proinflammatory cytokines such as TNFα or IL-1 (e.g. NIK, IKK, or MAP kinase inhibitors); IL-1β converting enzyme inhibitors; TNFα converting enzyme inhibitors; T-cell signalling inhibitors such as kinase inhibitors; metalloproteinase inhibitors; sulfasalazine; azathioprine; 6-mercaptopurines; angiotensin converting enzyme inhibitors; soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-11, IL-13 and TGFβ). Preferred examples of therapeutic agents for Crohn's disease with which a compound of formula (I), (I-a), (I-b), (I-c), or (I-d) may be combined include the following: TNF antagonists, for example, anti-TNF antibodies, D2E7 (adalimumab), CA2 (infliximab), CDP 571, TNFR-Ig constructs, (p75TNFRIgG (etanercept) and p55TNFRIgG (LENERCEPT™) inhibitors and PDE4 inhibitors. A compound of formula (I), (I-a), (I-b), (I-c), or (I-d) may be combined with corticosteroids, for example, budenoside and dexamethasone; sulfasalazine, 5-aminosalicylic acid; olsalazine; and agents which interfere with synthesis or action of proinflammatory cytokines such as IL-1, for example, IL-1β converting enzyme inhibitors and IL-1ra; T cell signaling inhibitors, for example, tyrosine kinase inhibitors; 6-mercaptopurine; IL-11; mesalamine; prednisone; azathioprine; mercaptopurine; infliximab; methylprednisolone sodium succinate; diphenoxylate/atrop sulfate; loperamide hydrochloride; methotrexate; omeprazole; folate; ciprofloxacin/dextrose-water; hydrocodone bitartrate/apap; tetracycline hydrochloride; fluocinonide; metronidazole; thimerosal/boric acid; cholestyramine/sucrose; ciprofloxacin hydrochloride; hyoscyamine sulfate; meperidine hydrochloride; midazolam hydrochloride; oxycodone HCl/acetaminophen; promethazine hydrochloride; sodium phosphate; sulfamethoxazole/trimethoprim; celecoxib; polycarbophil; propoxyphene napsylate; hydrocortisone; multivitamins; balsalazide disodium; codeine phosphate/apap; colesevelam HCl; cyanocobalamin; folic acid; levofloxacin; methylprednisolone; natalizumab and interferon-gamma.

Non-limiting examples of therapeutic agents for multiple sclerosis with which a compound of formula (I), (I-a), (I-b), (I-c), or (I-d) may be co-administered include the following: corticosteroids; prednisolone; methylprednisolone; azathioprine; cyclophosphamide; cyclosporine; methotrexate; 4-aminopyridine; tizanidine; interferon-β1a (AVONEX®; Biogen); interferon-β1b (BETASERON®; Chiron/Berlex); interferon α-n3) (Interferon Sciences/Fujimoto), interferon-α (Alfa Wassermann/J&J), interferon β1A-IF (Serono/Inhale Therapeutics), Peginterferon α 2b (Enzon/Schering-Plough), Copolymer 1 (Cop-1; COPAXONE®; Teva Pharmaceutical Industries, Inc.); hyperbaric oxygen; intravenous immunoglobulin; cladribine; antibodies to or antagonists of other human cytokines or growth factors and their receptors, for example, TNF, LT, IL-1, IL-2, IL-6, IL-7, IL-8, IL-12, IL-23, IL-15, IL-16, EMAP-II, GM-CSF, FGF, and PDGF. A compound of formula (I), (I-a), (I-b), (I-c), or (I-d) may be combined with antibodies to cell surface molecules such as CD2, CD3, CD4, CD8, CD19, CD20, CD25, CD28, CD30, CD40, CD45, CD69, CD80, CD86, CD90 or their ligands. A compound of formula (I), (I-a), (I-b), (I-c), or (I-d) may also be combined with agents such as methotrexate, cyclosporine, FK506, rapamycin, mycophenolate mofetil, leflunomide, an S1P1 agonist, NSAIDs, for example, ibuprofen, corticosteroids such as prednisolone, phosphodiesterase inhibitors, adensosine agonists, antithrombotic agents, complement inhibitors, adrenergic agents, agents which interfere with signalling by proinflammatory cytokines such as TNFα or IL-1 (e.g., NIK, IKK, p38 or MAP kinase inhibitors), IL-1β converting enzyme inhibitors, TACE inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g. soluble p55 or p75 TNF receptors, sIL-1RI, sIL-1RII, sIL-6R) and antiinflammatory cytokines (e.g. IL-4, IL-10, IL-13 and TGFβ).

A compound of formula (I), (I-a), (I-b), (I-c), or (I-d) may also be co-administered with agents, such as alemtuzumab, dronabinol, daclizumab, mitoxantrone, xaliproden hydrochloride, fampridine, glatiramer acetate, natalizumab, sinnabidol, α-immunokine NNSO3, ABR-215062, AnergiX.MS, chemokine receptor antagonists, BBR-2778, calagualine, CPI-1189, LEM (liposome encapsulated mitoxantrone), THC.CBD (cannabinoid agonist), MBP-8298, mesopram (PDE4 inhibitor), MNA-715, anti-IL-6 receptor antibody, neurovax, pirfenidone allotrap 1258 (RDP-1258), sTNF-R1, talampanel, teriflunomide, TGF-beta2, tiplimotide, VLA-4 antagonists (for example, TR-14035, VLA4 Ultrahaler, Antegran-ELAN/Biogen), interferon gamma antagonists and IL-4 agonists.

Non-limiting examples of therapeutic agents for ankylosing spondylitis with which a compound of formula (I), (I-a), (I-b), (I-c), or (I-d) may be co-administered include the following: ibuprofen, diclofenac, misoprostol, naproxen, meloxicam, indomethacin, diclofenac, celecoxib, rofecoxib, sulfasalazine, methotrexate, azathioprine, minocyclin, prednisone, and anti-TNF antibodies, D2E7 (HUMIRA®), CA2 (infliximab), CDP 571, TNFR-Ig constructs, (p75TNFRIgG)(ENBREL®) and p55TNFRIgG (LENERCEPT®).

Non-limiting examples of therapeutic agents for asthma with which a compound of formula (I), (I-a), (I-b), (I-c), or (I-d) may be co-administered include the following: albuterol, salmeterol/fluticasone, montelukast sodium, fluticasone propionate, budesonide, prednisone, salmeterol xinafoate, levalbuterol HCl, albuterol sulfate/ipratropium, prednisolone sodium phosphate, triamcinolone acetonide, beclomethasone dipropionate, ipratropium bromide, azithromycin, pirbuterol acetate, prednisolone, theophylline anhydrous, methylprednisolone sodium succinate, clarithromycin, zafirlukast, formoterol fumarate, influenza virus vaccine, amoxicillin trihydrate, flunisolide, allergy injection, cromolyn sodium, fexofenadine hydrochloride, flunisolide/menthol, amoxicillin/clavulanate, levofloxacin, inhaler assist device, guaifenesin, dexamethasone sodium phosphate, moxifloxacin HCl, doxycycline hyclate, guaifenesin/d-methorphan, p-ephedrine/cod/chlorphenir, gatifloxacin, cetirizine hydrochloride, mometasone furoate, salmeterol xinafoate, benzonatate, cephalexin, pe/hydrocodone/chlorphenir, cetirizine HCl/pseudoephed, phenylephrine/cod/promethazine, codeine/promethazine, cefprozil, dexamethasone, guaifenesin/pseudoephedrine, chlorpheniramine/hydrocodone, nedocromil sodium, terbutaline sulfate, epinephrine, methylprednisolone, anti-IL-13 antibody, and metaproterenol sulfate.

Non-limiting examples of therapeutic agents for COPD with which a compound of formula (I), (I-a), (I-b), (I-c), or (I-d) may be co-administered include the following: albuterol sulfate/ipratropium, ipratropium bromide, salmeterol/fluticasone, albuterol, salmeterol xinafoate, fluticasone propionate, prednisone, theophylline anhydrous, methylprednisolone sodium succinate, montelukast sodium, budesonide, formoterol fumarate, triamcinolone acetonide, levofloxacin, guaifenesin, azithromycin, beclomethasone dipropionate, levalbuterol HCl, flunisolide, ceftriaxone sodium, amoxicillin trihydrate, gatifloxacin, zafirlukast, amoxicillin/clavulanate, flunisolide/menthol, chlorpheniramine/hydrocodone, metaproterenol sulfate, methylprednisolone, mometasone furoate, p-ephedrine/cod/chlorphenir, pirbuterol acetate, p-ephedrine/loratadine, terbutaline sulfate, tiotropium bromide, (R,R)-formoterol, TgAAT, cilomilast and roflumilast.

Non-limiting examples of therapeutic agents for psoriasis with which a compound of formula (I), (I-a), (I-b), (I-c), or (I-d) may be co-administered include the following: calcipotriene, clobetasol propionate, triamcinolone acetonide, halobetasol propionate, tazarotene, methotrexate, fluocinonide, betamethasone diprop augmented, fluocinolone acetonide, acitretin, tar shampoo, betamethasone valerate, mometasone furoate, ketoconazole, pramoxine/fluocinolone, hydrocortisone valerate, flurandrenolide, urea, betamethasone, clobetasol propionate/emoll, fluticasone propionate, azithromycin, hydrocortisone, moisturizing formula, folic acid, desonide, pimecrolimus, coal tar, diflorasone diacetate, etanercept folate, lactic acid, methoxsalen, he/bismuth subgal/znox/resor, methylprednisolone acetate, prednisone, sunscreen, halcinonide, salicylic acid, anthralin, clocortolone pivalate, coal extract, coal tar/salicylic acid, coal tar/salicylic acid/sulfur, desoximetasone, diazepam, emollient, fluocinonide/emollient, mineral oil/castor oil/na lact, mineral oil/peanut oil, petroleum/isopropyl myristate, psoralen, salicylic acid, soap/tribromsalan, thimerosal/boric acid, celecoxib, infliximab, cyclosporine, alefacept, efalizumab, tacrolimus, pimecrolimus, PUVA, UVB, sulfasalazine, ABT-874 and ustekinamab.

Non-limiting examples of therapeutic agents for psoriatic arthritis with which a compound of formula (I), (I-a), (I-b), (I-c), or (I-d) may be co-administered include the following: methotrexate, etanercept, rofecoxib, celecoxib, folic acid, sulfasalazine, naproxen, leflunomide, methylprednisolone acetate, indomethacin, hydroxychloroquine sulfate, prednisone, sulindac, betamethasone diprop augmented, infliximab, methotrexate, folate, triamcinolone acetonide, diclofenac, dimethylsulfoxide, piroxicam, diclofenac sodium, ketoprofen, meloxicam, methylprednisolone, nabumetone, tolmetin sodium, calcipotriene, cyclosporine, diclofenac sodium/misoprostol, fluocinonide, glucosamine sulfate, gold sodium thiomalate, hydrocodone bitartrate/apap, ibuprofen, risedronate sodium, sulfadiazine, thioguanine, valdecoxib, alefacept, D2E7 (adalimumab), and efalizumab.

Examples of therapeutic agents for SLE (Lupus) with which a compound of formula (I), (I-a), (I-b), (I-c), or (I-d) may be co-administered include the following: NSAIDS, for example, diclofenac, naproxen, ibuprofen, piroxicam, indomethacin; COX2 inhibitors, for example, celecoxib, rofecoxib, valdecoxib; anti-malarials, for example, hydroxychloroquine; steroids, for example, prednisone, prednisolone, budenoside, dexamethasone; cytotoxics, for example, azathioprine, cyclophosphamide, mycophenolate mofetil, methotrexate; inhibitors of PDE4 or purine synthesis inhibitor, for example Cellcept®. A compound of formula (I), (I-a), (I-b), (I-c), or (I-d) may also be combined with agents such as sulfasalazine, 5-aminosalicylic acid, olsalazine, Imuran® and agents which interfere with synthesis, production or action of proinflammatory cytokines such as IL-1, for example, caspase inhibitors like IL-1β converting enzyme inhibitors and IL-1ra. A compound of formula (I), (I-a), (I-b), (I-c), or (I-d) may also be used with T cell signaling inhibitors, for example, tyrosine kinase inhibitors; or molecules that target T cell activation molecules, for example, CTLA-4-IgG or anti-B7 family antibodies, anti-PD-1 family antibodies. A compound of formula (I), (I-a), (I-b), (I-c), or (I-d) may be combined with IL-11 or anti-cytokine antibodies, for example, fonotolizumab (anti-IFNg antibody), or anti-receptor receptor antibodies, for example, anti-IL-6 receptor antibody and antibodies to B-cell surface molecules. A compound of formula (I), (I-a), (I-b), (I-c), or (I-d) may also be used with LW 394 (abetimus), agents that deplete or inactivate B-cells, for example, Rituximab (anti-CD20 antibody), lymphostat-B (anti-BlyS antibody), TNF antagonists, for example, anti-TNF antibodies, D2E7 (adalimumab), CA2 (infliximab), CDP 571, TNFR-Ig constructs, (p75TNFRIgG (etanercept) and p55TNFRIgG (LENERCEPT™).

The compounds of the invention can also be co-administered with a therapeutically effective amount of at least one additional therapeutic agents used in the prevention or treatment of AIDS, where examples of the agents include, HIV reverse transcriptase inhibitors, HIV protease inhibitors, immunomodulators, and other retroviral drugs. Examples of reverse transcriptase inhibitors include, but are not limited to, abacavir, adefovir, didanosine, dipivoxil delavirdine, efavirenz, lamivudine, nevirapine, stavudine zalcitabine, and zidovudine. Examples of protease inhibitors include, but are not limited to, amprenavir, indinavir, lopinavir, nelfinavir, ritonavir, and saquinavir.

The following Examples may be used for illustrative purposes and should not be deemed to narrow the scope of the invention.

### EXAMPLES

All reagents were of commercial grade and were used as received without further purification, unless otherwise stated. Commercially available anhydrous solvents were used for reactions conducted under inert atmosphere. Reagent grade solvents were used in all other cases, unless otherwise specified. Chemical shifts (δ) for ¹H NMR spectra were reported in parts per million (ppm) relative to tetramethylsilane (δ 0.00) or the appropriate residual solvent peak, i.e. CHCl₃ (δ 7.27), as internal reference. Multiplicities were given as singlet (s), doublet (d), triplet (t), quartet (q), quintuplet (quin), multiplet (m) and broad (br).

### Example 1

### rac-N-ethyl-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide

### Example 1A

### (E)-2-(5-bromo-2-methoxy-3-nitropyridin-4-yl)-N,N-dimethylethenamine

5-Bromo-2-methoxy-4-methyl-3-nitropyridine (15.0 g, 60.7 mmol, Pharmablock) was dissolved in N,N-dimethylformamide (300 mL), and lithium methanolate (6.07 mL, 6.07 mmol, 1 M) was added. The reaction mixture was heated to 100° C. To this mixture was added 1,1-dimethoxy-N,N-dimethylmethanamine (64.5 mL, 486 mmol) over 10 minutes. The reaction mixture was stirred at 95° C. for 16 hours. The reaction mixture was cooled to ambient temperature and water was added carefully (300 mL, exothermic). The resulting precipitate was collected by vacuum filtration, washed with water, and dried to provide the title compound (13.9 g, 45.9 mmol, 76% yield). ¹H NMR (400 MHz, DMSO-d₆) δ 8.21 (s, 1H), 7.02 (d, J=13.5 Hz, 1H), 4.77 (d, J=13.5 Hz, 1H), 3.85 (s, 3H), 2.87 (s, 6H); MS (ESI+) m/z 302.0 (M+H)⁺.

### Example 1B

### 4-bromo-7-methoxy-1H-pyrrolo[2,3-c]pyridine

Example 1A (13.9 g, 45.8 mmol) and ethyl acetate (150 mL) were added to Ra-Ni 2800 (pre-washed with ethanol) water slurry (6.9 g, 118 mmol) in a stainless steel pressure bottle and stirred for 30 minutes at 30 psi of hydrogen and ambient temperature. The reaction mixture was filtered and concentrated. The residue was triturated with dichloromethane, and the solid filtered to provide the title compound (5.82 g). The mother liquor was concentrated and the residue triturated again with dichloromethane and filtered to provide an additional 1.63 g of the title compound. Total yield was 7.45 g (72% yield). ¹H NMR (501 MHz, DMSO-d₆) δ 12.12 (s, 1H), 7.73 (s, 1H), 7.53 (d, J=2.9 Hz, 1H), 6.41 (d, J=2.9 Hz, 1H), 3.99 (s, 3H); MS (DCI+) m/z 226.8 (M+H)⁺.

### Example 1C

### 4-bromo-7-methoxy-1-tosyl-1H-pyrrolo[2,3-c]pyridine

A solution of Example 1B (7.42 g, 32.7 mmol) in N,N-dimethylformamide (235 mL) was stirred at ambient temperature. To this solution was added sodium hydride (1.18 g, 1.96 g of 60% dispersion in oil, 49.0 mmol), and the reaction mixture was stirred for 10 minutes. p-Toluenesulfonyl chloride (9.35 g, 49.0 mmol) was then added portion-wise, and the mixture was stirred at ambient temperature under nitrogen for 16 hours. The reaction mixture was quenched carefully with water and the resulting beige solid collected by vacuum filtration on a Buchner funnel, and washed with water. The solid was collected and dried in a vacuum oven at 50° C. to provide the title compound (12.4 g, 100% yield). ¹H NMR (400 MHz, DMSO-d₆) δ 8.15 (d, J=3.7 Hz, 1H), 7.96 (s, 1H), 7.88-7.77 (m, 2H), 7.42 (d, J=8.1 Hz, 2H), 6.77 (d, J=3.7 Hz, 1H), 3.80 (s, 3H), 2.35 (s, 3H); MS (ESI+) m/z 383.0 (M+H)⁺.

### Example 1D

### ethyl 4-bromo-7-methoxy-1-tosyl-1H-pyrrolo[2,3-c]pyridine-2-carboxylate

To a solution of Example 1C (10.5 g, 27.5 mmol) in tetrahydrofuran (170 mL) was added dropwise lithium N,N-diisopropylamide (tetrahydrofuran, 20.7 mL, 41.4 mmol) at −70° C. and then stirred between −70° C. and −50° C. for 45 minutes. Ethyl carbonochloridate (4.48 g, 41.3 mmol) was added dropwise and the mixture then was stirred at −70° C. for 1.5 hours. The reaction mixture was quenched with 20% aqueous ammonium chloride and extracted with ethyl acetate (150 mL). The organic layer was dried over Na₂SO₄, filtered, and concentrated under vacuum to give the crude product, which was washed with dichloromethane to afford the title compound (10 g, 80%) as a solid. ¹H NMR (400 MHz, DMSO-d₆) δ 8.17-8.03 (m, 3H), 7.59-7.46 (m, 2H), 7.25 (s, 1H), 4.38 (q, J=7.1 Hz, 2H), 3.82 (s, 3H), 2.41 (s, 3H), 1.33 (t, J=7.1 Hz, 3H); MS (ESI+) m/z 452.9 (M+H)⁺.

### Example 1E

### ethyl 4-bromo-7-oxo-1-tosyl-6,7-dihydro-1H-pyrrolo[2,3-c]pyridine-2-carboxylate

To a mixture of Example 1D (32.5 g, 71.7 mmol) and sodium iodide (16.12 g, 108 mmol) in CH₃CN (554 mL) was added chlorotrimethylsilane (11.68 g, 108 mmol) dropwise at ambient temperature. The resulting mixture was stirred at ambient temperature for 1 hour, then water (0.685 g, 38.0 mmol) was added dropwise and stirring continued at 65° C. for 3 hours. The reaction mixture was cooled to ambient temperature and filtered. The precipitate was dissolved in dichloromethane. The mixture was filtered again and the combined filtrate was concentrated under reduced pressure to give a brown solid which was washed with petroleum and dichloromethane to afford the title compound (23 g, 52.4 mmol, 73% yield) as solid. ¹H NMR (400 MHz, DMSO-d₆) δ 11.76 (s, 1H), 8.28-8.17 (m, 2H), 7.51-7.41 (m, 3H), 6.99 (s, 1H), 4.35 (q, J=7.1 Hz, 2H), 2.39 (s, 3H), 1.31 (t, J=7.1 Hz, 3H); MS (ESI+) m/z 440.9 (M+H)⁺.

### Example 1F

### ethyl 4-bromo-6-methyl-7-oxo-1-tosyl-6,7-dihydro-1H-pyrrolo[2,3-c]pyridine-2-carboxylate

To a solution of Example 1E (7.5 g, 17.07 mmol) in tetrahydrofuran (100 mL) was added sodium hydride (60% dispersion in mineral oil, 0.520 g, 21.68 mmol) in portions at 0° C. The mixture was stirred for 30 minutes, then iodomethane (3.64 g, 25.6 mmol) was added dropwise at 0° C. and the resulting mixture was stirred at ambient temperature for 3 hours. Another portion of iodomethane (3.64 g, 25.6 mmol) was added at 0° C. and the reaction mixture was stirred at ambient temperature for 12 hours. The reaction was quenched with 20% aqueous ammonium chloride and extracted with ethyl acetate three times. The combined extracts were dried over Na₂SO₄, filtered, and concentrated under reduced pressure. This material was purified by flash chromatography (silica gel, 0-50% ethyl acetate/heptanes) to afford the crude product as a yellow solid, which was washed with methanol to obtain the title compound (15.3 g, 80% yield) as solid. ¹H NMR (400 MHz, DMSO-d₆) δ 8.33-8.22 (m, 2H), 7.91 (s, 1H), 7.50 (d, J=8.1 Hz, 2H), 7.02 (s, 1H), 4.37 (q, J=7.1 Hz, 2H), 3.43 (s, 3H), 2.41 (s, 3H), 1.32 (t, J=7.1 Hz, 3H); LC/MS (ESI+) m/z 450.0 (M+H)⁺.

### Example 1G

### ethyl 6-methyl-7-oxo-1-tosyl-4-vinyl-6,7-dihydro-1H-pyrrolo[2,3-c]pyridine-2-carboxylate

Example 1F (2.72 g, 6 mmol), Pd₂(dba)₃ (0.055 g, 0.060 mmol), 1,3,5,7-tetramethyl-6-phenyl-2,4,8-trioxa-6-phophaadamantane (0.042 g, 0.144 mmol), and potassium phosphate tribasic (3.18 g, 15.00 mmol) were combined in a 40 mL vial with stir bar and septum cap. The vial was evacuated and backfilled with nitrogen and then charged with dioxane (24.00 mL), 4,4,5,5-tetramethyl-2-vinyl-1,3,2-dioxaborolane (1.323 mL, 7.80 mmol, Aldrich), and water (6.00 mL). The resulting mixture was then heated to 80° C. After 18 hours the reaction was diluted with ethyl acetate and washed with water and brine. The organic phase was then dried with MgSO₄, filtered, and concentrated under reduced pressure. The crude residue was purified via flash chromatography (ISCO Combiflash, 0-100% ethyl acetate/heptanes, 120 g Redisep silica column) to yield the title compound (1.34 g, 3.35 mmol, 55.8% yield) as solid. ¹H NMR (501 MHz, CDCl₃) δ 8.59-8.45 (m, 2H), 7.49-7.37 (m, 2H), 7.18 (d, J=0.7 Hz, 1H), 7.13 (s, 1H), 6.60 (ddd, J=17.7, 11.2, 0.8 Hz, 1H), 5.61 (dd, J=17.7, 0.7 Hz, 1H), 5.29 (dd, J=11.2, 0.8 Hz, 1H), 4.49 (q, J=7.2 Hz, 2H), 3.61 (s, 3H), 2.47 (s, 3H), 1.46 (t, J=7.1 Hz, 3H); LC/MS (ESI+) m/z 401.0 (M+H)⁺.

### Example 1H

### rac-ethyl 6-methyl-1-(4-methylbenzene-1-sulfonyl)-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxylate

A solution of 3-diazo-1-methylindolin-2-one (180 mg, 1.039 mmol, prepared as described in Org. Lett., 2016, 18, 1358-1361) in dichloromethane (5 mL) was added via syringe pump over 12 hours to a solution of Example 1G (362 mg, 0.904 mmol) and rhodium(II) acetate dimer (3.99 mg, 9.04 μmol) in dichloromethane (13 mL) at 45° C. After stirring at 45° C. for an additional 6 hours the reaction mixture was concentrated and the crude residue was purified via flash chromatography (ISCO Combiflash, 50-100% ethyl acetate/heptanes, 40 g Redisep silica column) to yield the title compound (463 mg, 0.849 mmol, 94% yield, single diastereomer) as a solid. ¹H NMR (400 MHz, CDCl₃) δ 8.53-8.37 (m, 2H), 7.40 (d, J=8.2 Hz, 2H), 7.22 (td, J=7.7, 1.2 Hz, 1H), 6.98 (d, J=1.5 Hz, 1H), 6.92 (d, J=7.8 Hz, 1H), 6.77 (td, J=7.6, 1.0 Hz, 1H), 6.55 (d, J=0.9 Hz, 1H), 6.23 (d, J=7.4 Hz, 1H), 5.32 (d, J=0.9 Hz, 1H), 4.41 (q, J=7.1 Hz, 2H), 3.58 (s, 3H), 3.38 (s, 3H), 3.05 (ddd, J=9.1, 7.7, 1.5 Hz, 1H), 2.46 (s, 3H), 2.24 (dd, J=9.0, 4.5 Hz, 1H), 1.83 (dd, J=7.8, 4.5 Hz, 1H), 1.43-1.38 (m, 3H); MS (ESI+) m/z 546.1 (M+H)⁺.

### Example 1I

### rac-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxylic acid

An aqueous solution of sodium hydroxide (21.44 mL, 1 M, 21.44 mmol) was added to Example 1H (3.9 g, 7.15 mmol) in dioxane (50 mL) and the reaction mixture was heated to 80° C. After 90 minutes the reaction was cooled in an ice bath and 400 mL of cold 1 M HCl was added to form a tan precipitate. The mixture was filtered and the collected solid was washed with cold water and dried in a vacuum oven to obtain the title compound (2.26 g, 6.22 mmol, 87% yield) as a solid. ¹H NMR (501 MHz, DMSO-d₆) δ 12.93 (s, 1H), 12.52 (d, J=2.3 Hz, 1H), 7.43 (d, J=1.3 Hz, 1H), 7.09 (td, J=7.7, 1.2 Hz, 1H), 7.00-6.97 (m, 1H), 6.68 (td, J=7.5, 1.0 Hz, 1H), 6.40 (dd, J=7.5, 1.1 Hz, 1H), 6.31 (d, J=2.1 Hz, 1H), 3.55 (d, J=1.1 Hz, 3H), 3.26 (s, 3H), 2.92 (ddd, J=9.1, 7.8, 1.3 Hz, 1H), 2.24 (dd, J=7.9, 4.6 Hz, 1H), 1.98 (dd, J=9.0, 4.6 Hz, 1H); MS (ESI+) m/z 364.1 (M+H)⁺.

### Example 1J

### rac-N-ethyl-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide

N,N-Diisopropylethylamine (0.048 mL, 0.275 mmol) was added to a mixture of Example 1I (40 mg, 0.110 mmol) and HATU (50.2 mg, 0.132 mmol) in N,N-dimethylformamide (1 mL) at 0° C. After stirring for 30 minutes ethylamine (0.110 mL, 0.220 mmol, 2M in tetrahydrofuran) was added in one portion and the reaction mixture was warmed to room temperature. After 18 hours the reaction mixture was diluted with ethyl acetate and washed with a saturated aqueous NaHCO₃ solution and brine. The organic phase was then dried with MgSO₄, filtered, and concentrated under reduced pressure. The crude residue was purified via flash chromatography (ISCO Combiflash, 2-6% methanol/dichloromethane, 12 g Redisep Gold silica column) to yield the title compound (22.9 mg, 0.059 mmol, 53.3% yield) as a solid. ¹H NMR (400 MHz, DMSO-d₆) δ 12.10 (s, 1H), 8.26 (t, J=5.4 Hz, 1H), 7.42 (d, J=1.3 Hz, 1H), 7.08 (td, J=7.7, 1.2 Hz, 1H), 6.98 (d, J=7.8 Hz, 1H), 6.68 (td, J=7.5, 1.0 Hz, 1H), 6.50-6.36 (m, 2H), 3.56 (s, 3H), 3.26 (s, 3H), 3.18 (pt, J=7.1, 3.6 Hz, 2H), 2.92 (td, J=8.5, 8.0, 1.3 Hz, 1H), 2.26 (dd, J=7.8, 4.6 Hz, 1H), 1.97 (dd, J=8.9, 4.7 Hz, 1H), 1.05 (t, J=7.2 Hz, 3H); MS (ESI−) m/z 388.9 (M−H)⁻.

### Example 2

### rac-N-cyclopropyl-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide

Aqueous NaOH (0.134 mL, 2.54 mmol, 50% in water) was added to a solution of Example 1H (462 mg, 0.847 mmol) in dioxane (4.2 mL) and water (1.4 mL). The reaction was then heated at 90° C. for 1 hour. After cooling to room temperature the reaction was acidified with 1M HCl and extracted three times with ethyl acetate. The combined organic phases were then washed with brine, dried with MgSO₄, filtered, and concentrated under reduced pressure. The resulting residue was taken up in N,N-dimethylformamide (6 mL) and cooled in an ice bath. HATU (386 mg, 1.016 mmol) and N,N-diisopropylethylamine (0.222 mL, 1.270 mmol) were added, followed after 30 minutes by cyclopropylamine (72.5 mg, 1.270 mmol). After 1 hour the reaction mixture was diluted with ethyl acetate and washed with a saturated aqueous NaHCO₃ solution and brine. The organic phase was then dried with MgSO₄, filtered, and concentrated under reduced pressure. The crude residue was then purified via reverse phase HPLC (5-95% acetonitrile/water with 0.1% trifluoroacetic acid) to yield the title compound (69 mg, 0.171 mmol, 20.25% yield) as a solid. ¹H NMR (400 MHz, DMSO-d₆) δ 12.04 (d, J=2.2 Hz, 1H), 8.25 (d, J=4.1 Hz, 1H), 7.40 (d, J=1.2 Hz, 1H), 7.07 (td, J=7.7, 1.2 Hz, 1H), 6.96 (d, J=7.6 Hz, 1H), 6.66 (td, J=7.6, 1.0 Hz, 1H), 6.46-6.33 (m, 2H), 3.53 (s, 3H), 3.24 (s, 3H), 2.89 (td, J=8.4, 7.9, 1.3 Hz, 1H), 2.70 (tt, J=7.7, 3.9 Hz, 1H), 2.24 (dd, J=7.9, 4.7 Hz, 1H), 1.95 (dd, J=9.0, 4.6 Hz, 1H), 0.68-0.59 (m, 2H), 0.49-0.39 (m, 2H); MS (ESI−) m/z 401.3 (M−H)⁻.

### Example 3

### N-cyclopropyl-6-methyl-4-[(1S,2R)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide

The enantiomers of Example 2 (330 mg) were separated by preparative SFC on a THAR/Waters SFC 80 system. The mobile phase comprised supercritical CO₂ with a modifier of methanol buffered with 0.1% diethylamine at a flow rate of 70 g/min. The column was at ambient temperature and the backpressure regulator was set to maintain 100 bar. The sample was dissolved in methanol at a concentration of 15 mg/mL. The sample was loaded into the modifier stream in 1 mL (15 mg) injections. The mobile phase was held isocratically at 30% methanol:CO₂. Fraction collection was time triggered. The instrument was fitted with a Whelk-O (S,S) column with dimensions 21 mm i.d.×250 mm length with 5 μm particles. The title compound (83.9 mg, 0.208 mmol, 25.4% yield) was isolated as a solid with a retention time of 9.1 minutes. ¹H NMR (400 MHz, DMSO-d₆) δ 12.04 (d, J=2.2 Hz, 1H), 8.25 (d, J=4.1 Hz, 1H), 7.40 (d, J=1.2 Hz, 1H), 7.07 (td, J=7.7, 1.2 Hz, 1H), 6.96 (d, J=7.6 Hz, 1H), 6.66 (td, J=7.6, 1.0 Hz, 1H), 6.46-6.33 (m, 2H), 3.53 (s, 3H), 3.24 (s, 3H), 2.89 (td, J=8.4, 7.9, 1.3 Hz, 1H), 2.70 (tt, J=7.7, 3.9 Hz, 1H), 2.24 (dd, J=7.9, 4.7 Hz, 1H), 1.95 (dd, J=9.0, 4.6 Hz, 1H), 0.68-0.59 (m, 2H), 0.49-0.39 (m, 2H); MS (ESI−) m/z 401.3 (M−H)⁻.

### Example 4

### N-cyclopropyl-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide

The title compound (90.6 mg, 0.225 mmol, 27.5% yield) was isolated as a solid with a retention time of 10.4 minutes from the preparative SFC purification as described in Example 3. ¹H NMR (400 MHz, DMSO-d₆) δ 12.04 (d, J=2.2 Hz, 1H), 8.25 (d, J=4.1 Hz, 1H), 7.40 (d, J=1.2 Hz, 1H), 7.07 (td, J=7.7, 1.2 Hz, 1H), 6.96 (d, J=7.6 Hz, 1H), 6.66 (td, J=7.6, 1.0 Hz, 1H), 6.46-6.33 (m, 2H), 3.53 (s, 3H), 3.24 (s, 3H), 2.89 (td, J=8.4, 7.9, 1.3 Hz, 1H), 2.70 (tt, J=7.7, 3.9 Hz, 1H), 2.24 (dd, J=7.9, 4.7 Hz, 1H), 1.95 (dd, J=9.0, 4.6 Hz, 1H), 0.68-0.59 (m, 2H), 0.49-0.39 (m, 2H); MS (ESI−) m/z 401.3 (M−H)⁻.

### Example 5

### rac-N-(3,3-difluorocyclobutyl)-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide

N,N-Diisopropylethylamine (0.036 mL, 0.206 mmol) was added to a mixture of Example 1I (30 mg, 0.083 mmol) and HATU (37.7 mg, 0.099 mmol) in N,N-dimethylformamide (0.83 mL) at 0° C. 3,3-Difluorocyclobutanamine hydrochloride (15.41 mg, 0.107 mmol, Aldrich) was added in one portion after 20 minutes. After stirring for 18 hours the reaction mixture was concentrated and the crude residue purified via reverse phase HPLC (5-95% acetonitrile/water with 0.1% trifluoroacetic acid) to yield the title compound (19.6 mg, 0.043 mmol, 52.5% yield) as a solid. ¹H NMR (501 MHz, DMSO-d₆) δ 12.19 (d, J=2.2 Hz, 1H), 8.66 (d, J=6.5 Hz, 1H), 7.45 (d, J=1.3 Hz, 1H), 7.09 (td, J=7.7, 1.2 Hz, 1H), 6.99 (dd, J=8.0, 1.0 Hz, 1H), 6.69 (td, J=7.5, 1.0 Hz, 1H), 6.47 (d, J=2.2 Hz, 1H), 6.43 (dd, J=7.4, 1.1 Hz, 1H), 4.15 (p, J=7.1 Hz, 1H), 3.57 (s, 3H), 3.26 (s, 3H), 2.99-2.84 (m, 3H), 2.71-2.54 (m, 2H), 2.28 (dd, J=7.9, 4.7 Hz, 1H), 1.98 (dd, J=9.0, 4.6 Hz, 1H); MS (ESI+) m/z 453.1 (M+H)⁺.

### Example 6

### 6-methyl-4-[(1S,2R)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxylic acid

The product from Example 1I was purified by chiral chromatography on a YMC-SB column eluting with 18% methanol in carbon dioxide. The absolute stereochemistry of the title compound was arbitrarily assigned to the first eluting enantiomer. ¹H NMR (400 MHz, DMSO-d₆) δ 10.38 (s, br, 1H), 7.34 (s, 1H), 7.06 (t, J=7.7 Hz, 1H), 6.96 (d, J=7.7 Hz, 1H), 6.66 (t, J=7.5 Hz, 1H), 6.40 (d, J=7.4 Hz, 1H), 6.03 (s, 1H), 3.51 (s, 3H), 3.24 (s, 3H), 2.89 (t, J=8.8 Hz, 1H), 2.21 (dd, J=7.9, 4.6 Hz, 1H), 1.94 (dd, J=9.0, 4.5 Hz, 1H). (ESI+) m/z 364 (M+H)⁺.

### Example 7

### 6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxylic acid

The product from Example 1I was purified by chiral chromatography on a YMC-SB column eluting with 18% methanol in carbon dioxide. The absolute stereochemistry of the title compound was arbitrarily assigned to the second eluting enantiomer. ¹H NMR (400 MHz, DMSO-d₆) δ 9.86 (s, br, 1H), 7.34 (d, J=1.3 Hz, 1H), 7.10-7.05 (m, 1H), 6.97 (d, J=7.7 Hz, 1H), 6.71-6.65 (m, 1H), 6.41 (dd, J=7.5, 1.1 Hz, 1H), 5.97 (s, 1H), 3.53 (s, 3H), 3.26 (s, 3H), 2.95-2.86 (m, 1H), 2.22 (dd, J=8.0, 4.5 Hz, 1H), 1.95 (dd, J=9.0, 4.5 Hz, 1H). MS (ESI+) m/z 364 (M+H)⁺.

### Example 8

### rac-6-methyl-4-[(1R,2S)-1′-methyl-2′-oxo-1′,2′-dihydrospiro[cyclopropane-1,3′-indol]-2-yl]-7-oxo-N-phenyl-6,7-dihydro-1H-pyrrolo[2,3-c]pyridine-2-carboxamide

To a solution of Example 1I (20 mg. 0.06 mmol) in dichloromethane (1 mL) at room temperature was added 1-chloro-N,N-2-trimethylprop-1-en-1-amine (14.71 mg, 0.11 mmol). The solution was stirred for 10 minutes. To this solution was added a solution of aniline (20.5 mg, 0.22 mmol) in pyridine (1 mL). The reaction mixture was stirred for 10 minutes and then concentrated. Purification via reverse phase HPLC (X-Select CSH™ Preparative C18 column, 5 μm, OBD 30× 100 mm, 5-95% acetonitrile/water with 0.1% trifluoroacetic acid) followed by flash chromatography (0-10% methanol/dichloromethane) provided the title compound (9.7 mg, 0.02 mmol, 40.2%). ¹H NMR (400 MHz, DMSO-d₆) δ 12.44 (s, 1H), 10.06 (s, 1H), 7.68-7.62 (m, 2H), 7.47 (s, 1H), 7.35-7.28 (m, 2H), 7.11-7.03 (m, 2H), 7.01-6.95 (m, 1H), 6.72-6.64 (m, 2H), 6.44 (d, J=1.2, 7.5 Hz, 1H), 3.58 (s, 3H), 3.27 (s, 3H), 2.96 (t, J=8.6 Hz, 1H), 2.31-2.25 (m, 1H), 2.03-1.95 (m, 1H). MS (ESI) m/z 439.2 (M+H)⁺.

### Example 9

### rac-N-(4-methoxyphenyl)-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide

The title compound was prepared similarly to the procedure described in Example 8, substituting 4-methoxyaniline for aniline (10.5 mg, 0.02 mmol, 40.7% yield). ¹H NMR (400 MHz, DMSO-d₆) δ 12.36 (s, 1H), 9.96 (s, 1H), 7.58-7.53 (m, 2H), 7.47-7.45 (m, 1H), 7.09 (td, J=1.2, 7.7 Hz, 1H), 6.98 (d, J=7.8 Hz, 1H), 6.91-6.86 (m, 2H), 6.69 (td, J=1.1, 7.6 Hz, 1H), 6.63 (s, 1H), 6.44 (dd, J=1.2, 7.6 Hz, 1H), 3.71 (s, 3H), 3.57 (s, 3H), 3.27 (s, 3H), 2.95 (ddd, J=1.3, 7.8, 9.1 Hz, 1H), 2.28 (dd, J=4.6, 7.8 Hz, 1H), 1.99 (dd, J=4.6, 8.9 Hz, 1H). MS (ESI) m/z 469.1 (M+H)⁺.

### Example 10

### rac-N-[3-(hydroxymethyl)phenyl]-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide

The title compound was prepared similarly to the procedure described in Example 8 substituting (3-aminophenyl)methanol for aniline (10.7 mg, 0.02 mmol, 41.5%). ¹H NMR (400 MHz, DMSO-d₆) δ 12.42 (s, 1H), 10.05 (s, 1H), 7.61 (d, J=1.7 Hz, 1H), 7.59-7.55 (m, 1H), 7.47 (d, J=1.4 Hz, 1H), 7.25 (t, J=7.8 Hz, 1H), 7.09 (td, J=1.2, 7.7 Hz, 1H), 6.99 (t, J=6.9 Hz, 2H), 6.72-6.65 (m, 2H), 6.44 (dd, J=1.2, 7.6 Hz, 1H), 5.21 (t, J=5.7 Hz, 1H), 4.46 (d, J=5.6 Hz, 2H), 3.58 (s, 3H), 3.27 (s, 3H), 2.96 (td, J=1.2, 7.7, 8.4 Hz, 1H), 2.28 (dd, J=4.7, 7.9 Hz, 1H), 1.99 (dd, J=4.6, 8.9 Hz, 1H). MS (ESI) m/z 469.1 (M+H)⁺.

### Example 11

### rac-N-cyclopropyl-4-[(1R,2S)-6′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

### Example 11A

### 6-fluoro-1-methylindoline-2,3-dione

A solution of 6-fluoroindoline-2,3-dione (1 g, 6.06 mmol, SCRC) in anhydrous N,N-dimethylformamide (40 mL) was cooled to 0° C., whereupon NaH (60% dispersion in mineral oil, 0.283 g, 7.09 mmol) was added in one portion and the reaction was stirred for 5 minutes. Iodomethane (0.443 mL, 7.09 mmol) was added and the reaction was stirred at 0° C. for 30 minutes. The mixture was then poured into saturated aqueous NH₄Cl and extracted with ethyl acetate. The combined organic portions were washed with water and brine, dried (MgSO₄), filtered, and concentrated to give the crude product (1.2 g, 88% purity), which was used without further purification.

### Example 11B

### 3-diazo-6-fluoro-1-methylindolin-2-one

To a stirring suspension of 4-methylbenzenesulfonohydrazide (1.247 g, 6.70 mmol, SCRC) in methanol (30 mL) at room temperature was added Example 11A (1.2 g, 6.70 mmol) in one portion. All the material dissolved after the addition and a yellow precipitate began to form within 10 minutes. After 3 hours the reaction was concentrated to a yellow solid. To this were added N-benzyl-N,N-diethylethanaminium chloride (0.031 g, 0.134 mmol, SCRC), dichloromethane (12 mL) and aqueous NaOH (12 mL, 4.00 mmol, 1M in water). The resulting mixture was stirred vigorously and heated at 40° C. for 15 hours, then the reaction mixture was diluted with ethyl acetate and water and the layers were separated. The organic phase was washed with water and brine, dried with MgSO₄, filtered, and concentrated under reduced pressure. The residue was then purified via flash chromatography (ISCO Combiflash, 0-100% ethyl acetate/heptanes, 40 g Redisep silica column) to yield the title compound (0.448 g, 2.344 mmol, 35% yield) as a solid.

### Example 11C

### rac-ethyl 4-[(1R,2S)-6′-fluoro-′-methyl-2′-oxo-1′,2′-dihydrospiro[cyclopropane-1,3′-indol]-2-yl]-6-methyl-1-(4-methylbenzene-1-sulfonyl)-7-oxo-6,7-dihydro-1H-pyrrolo[2,3-c]pyridine-2-carboxylate

A solution of Example 11B (119 mg, 0.624 mmol) in dichloromethane (5 mL) was added over 12 hours via syringe pump through a reflux condenser to a solution of Example 1G (250 mg, 0.624 mmol) and rhodium(II) acetate dimer (2.76 mg, 6.24 μmol) in dichloromethane (10 mL) at 45° C. After an additional 6 hours the reaction was concentrated and the crude residue was purified via flash chromatography (ISCO Combiflash, 50-100% ethyl acetate/heptanes, 40 g Redisep silica column) to provide the title compound (150 mg, 0.266 mmol, 42.6% yield).

### Example 11D

### rac-N-cyclopropyl-4-[(1R,2S)-6′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

Aqueous NaOH (0.134 mL, 2.54 mmol, 50% in water) was added to a solution of Example 11C (200 mg, 0.355 mmol) in dioxane (4.2 mL) and water (1.4 mL). The reaction was then heated at 90° C. for 1 hour. After cooling to room temperature the reaction was acidified with 1M HCl and extracted three times with ethyl acetate. The combined organic phases were washed with brine, dried with MgSO₄, filtered, and concentrated under reduced pressure. The resulting residue was taken up in N,N-dimethylformamide (6 mL), cooled in an ice bath, and treated with HATU (162 mg, 0.426 mmol) and N,N-diisopropylethylamine (0.093 mL, 0.532 mmol). After 30 minutes cyclopropanamine (30.4 mg, 0.532 mmol) was added, and then after 15 minutes the reaction mixture was warmed to room temperature and stirred for an additional 1 hour. The reaction mixture was diluted with ethyl acetate, washed with saturated NaHCO₃ and brine, dried with Na₂SO₄, filtered, and concentrated under reduced pressure. The crude residue was purified by reverse phase HPLC (Waters)(Bridge 21×250 mm column, gradient 25-55% acetonitrile/10 mM NH₄HCO₃, flow rate 25 mL/minute) to afford the title compound (60.2 mg, 0.143 mmol, 40.3% yield). ¹H NMR (400 MHz, CDCl₃) δ 7.89 (s, 1H), 6.91 (s, 1H), 6.68 (s, 1H), 6.62 (dd, J=8.8, 2.1 Hz, 1H), 6.41-6.31 (m, 1H), 6.12 (dd, J=8.2, 5.2 Hz, 1H), 3.74 (s, 3H), 3.34 (s, 3H), 3.14 (t, J=8.4 Hz, 1H), 2.78 (s, 1H), 2.21 (dd, J=9.0, 4.6 Hz, 1H), 1.90 (dd, J=7.7, 4.7 Hz, 1H), 0.86 (t, J=6.2 Hz, 2H), 0.63 (s, 2H).

### Example 12

### rac-4-[(1R,2S)-6′-chloro-1′-methyl-2′-oxo-1′,2′-dihydrospiro[cyclopropane-1,3′-indol]-2-yl]-N-cyclopropyl-6-methyl-7-oxo-6,7-dihydro-1H-pyrrolo[2,3-c]pyridine-2-carboxamide

### Example 12A

### 6-chloro-1-methylindoline-2,3-dione

A solution of 6-chloroindoline-2,3-dione (1 g, 5.51 mmol, SCRC) in anhydrous N,N-dimethylformamide (40 mL) was cooled to 0° C., whereupon NaH (60% dispersion in mineral oil, 0.258 g, 6.44 mmol) was added in one portion and the reaction was stirred for 5 minutes. Iodomethane (0.403 mL, 6.44 mmol) was added and the reaction mixture was stirred at 0° C. for 30 minutes. The mixture was then poured into saturated aqueous NH₄Cl and extracted with ethyl acetate. The combined organic portions were washed with water and brine, dried (MgSO₄), filtered, and concentrated to give the crude product (1.2 g), which was used without further purification.

### Example 12B

### 6-chloro-3-diazo-1-methylindolin-2-one

To a stirring suspension of 4-methylbenzenesulfonohydrazide (1.143 g, 6.13 mmol, SCRC) in methanol (30 mL) at room temperature was added Example 12A (1.2 g, 6.13 mmol) in one portion. All the material dissolved after the addition and a yellow precipitate began to form within 10 minutes. After 3 hours the reaction mixture was concentrated to a yellow solid. To this were added N-benzyl-N,N-diethylethanaminium chloride (0.028 g, 0.123 mmol, SCRC), dichloromethane (12 mL) and aqueous NaOH (12 mL, 4.00 mmol, 1M in water). The resulting mixture was stirred vigorously and heated at 40° C. for 15 hours, then the reaction mixture was diluted with ethyl acetate and water and the layers were separated. The organic phase was washed with water and brine, dried with MgSO₄, filtered, and concentrated under reduced pressure. The residue was then purified via flash chromatography (ISCO Combiflash, 0-100% ethyl acetate/heptanes, 40 g Redisep silica column) to yield the title compound (0.524 g, 2.147 mmol, 35% yield) as a solid.

### Example 12C

### rac-ethyl 4-[(1R,2S)-6′-chloro-1′-methyl-2′-oxo-1′,2′-dihydrospiro[cyclopropane-1,3′-indol]-2-yl]-6-methyl-1-(4-methylbenzene-1-sulfonyl)-7-oxo-6,7-dihydro-1H-pyrrolo[2,3-c]pyridine-2-carboxylate

A solution of Example 12B (62.2 mg, 0.300 mmol) in dichloromethane (5 mL) was added over 12 hours via syringe pump through a reflux condenser to a solution of Example 1G (120 mg, 0.300 mmol) and rhodium(II) acetate dimer (1.32 mg, 3.00 μmol) in dichloromethane (10 mL) at 45° C. After an additional 6 hours the reaction mixture was concentrated and the crude residue was purified via flash chromatography (ISCO Combiflash, 50-100% ethyl acetate/heptanes, 40 g Redisep silica column) to provide the title compound (100 mg, 0.171 mmol, 57.2% yield).

### Example 12D

### rac-4-[(1R,2S)-6′-chloro-1′-methyl-2′-oxo-1′,2′-dihydrospiro[cyclopropane-1,3′-indol]-2-yl]-N-cyclopropyl-6-methyl-7-oxo-6,7-dihydro-1H-pyrrolo[2,3-c]pyridine-2-carboxamide

Aqueous NaOH (0.134 mL, 2.54 mmol, 50% in water) was added to a solution of Example 12C (120 mg, 0.207 mmol) in dioxane (4.2 mL) and water (1.4 mL). The reaction was then heated to 90° C. for 1 hour. After cooling to room temperature the reaction was acidified with 1M HCl and extracted three times with ethyl acetate. The combined organic phases were washed with brine, dried with MgSO₄, and concentrated under reduced pressure. The resulting residue was taken up in N,N-dimethylformamide (6 mL), cooled in an ice bath, and treated with HATU (94 mg, 0.248 mmol) and N,N-diisopropylethylamine (0.054 mL, 0.31 mmol). After 30 minutes cyclopropanamine (17.72 mg, 0.310 mmol) was added, and then after 15 minutes the reaction was warmed to room temperature and stirred for an additional 1 hour. The reaction was diluted with ethyl acetate, washed with saturated NaHCO₃ and brine, dried with Na₂SO₄ and concentrated under reduced pressure. The crude residue was purified by reverse phase HPLC (Waters XBridge 21×250 mm column, gradient 25-55% acetonitrile/10 mM NH₄HCO₃, flow rate 25 mL/minute) to afford the title compound (22.1mg, 0.048 mmol, 23.42% yield). ¹H NMR (400 MHz, CDCl₃) δ 12.17 (s, 2H), 8.11 (s, 2H), 6.92 (s, 2H), 6.87 (s, 2H), 6.73 (s, 2H), 6.64 (d, J=8.0 Hz, 2H), 6.10 (d, J=8.0 Hz, 2H), 3.76 (s, 6H), 3.34 (s, 6H), 3.17 (d, J=8.4 Hz, 2H), 2.78 (s, 2H), 2.23 (dd, J=8.9, 4.6 Hz, 2H), 1.93 (dd, J=7.5, 4.8 Hz, 3H), 0.85 (d, J=6.2 Hz, 4H), 0.63 (s, 4H).

### Example 13

### rac-N-cyclopropyl-4-[(1R,2S)-1′,6′-dimethyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

### Example 13A

### 1,6-dimethylindoline-2,3-dione

A solution of 6-methylindoline-2,3-dione (1 g, 6.21 mmol, SCRC) in anhydrous N,N-dimethylformamide (40 mL) was cooled to 0° C., whereupon NaH (60% dispersion in mineral oil, 0.290 g, 7.26 mmol) was added in one portion and the reaction was stirred for 5 minutes. Iodomethane (0.454 mL, 7.26 mmol) was added and the reaction was mixture stirred at 0° C. for 30 minutes. The mixture was then poured into saturated aqueous NH₄Cl and extracted with ethyl acetate. The combined organic portions were washed with water and brine, dried (MgSO₄), filtered, and concentrated to give the crude product (1.2 g), which was used without further purification.

### Example 13B

### 3-diazo-1,6-dimethylindolin-2-one

To a stirring suspension of 4-methylbenzenesulfonohydrazide (1.087 g, 5.84 mmol, SCRC) in methanol (30 mL) at room temperature was added Example 13A (1.023 g, 5.84 mmol) in one portion. All the material dissolved after the addition and a yellow precipitate began to form within 10 minutes. After 3 hours the reaction was concentrated to a yellow solid. To this were added N-benzyl-N,N-diethylethanaminium chloride (0.027 g, 0.117 mmol, SCRC), dichloromethane (12 mL) and aqueous NaOH (12 mL, 4.00 mmol, 1M in water). The resulting mixture was stirred vigorously and heated at 40° C. for 15 hours, then the reaction was diluted with ethyl acetate and water and the layers were separated. The organic phase was washed with water and brine, dried with MgSO₄, filtered, and concentrated under reduced pressure. The residue was then purified via flash chromatography (ISCO Combiflash, 0-100% ethyl acetate/heptanes, 40 g Redisep silica column) to yield the title compound (0.382 g, 2.043 mmol, 35% yield) as a solid.

### Example 13C

### rac-ethyl 4-[(1R,2S)-1′,6′-dimethyl-2′-oxo-1′,2′-dihydrospiro[cyclopropane-1,3′-indol]-2-yl]-6-methyl-1-(4-methylbenzene-1-sulfonyl)-7-oxo-6,7-dihydro-1H-pyrrolo[2,3-c]pyridine-2-carboxylate

A solution of Example 13B (56.1 mg, 0.300 mmol) in dichloromethane (5 mL) was added over 12 hours via syringe pump through a reflux condenser to a solution of Example 1G (120 mg, 0.300 mmol) and rhodium(II) acetate dimer (1.32 mg, 3.00 μmol) in dichloromethane (10 mL) at 45° C. After an additional 6 hours the reaction mixture was concentrated and the crude residue was purified via flash chromatography (ISCO Combiflash, 50-100% ethyl acetate/heptanes, 40 g Redisep silica column) to provide the title compound (120 mg, 0.214 mmol, 71.6% yield).

### Example 13D

### rac-N-cyclopropyl-4-[(1R,2S)-1′,6′-dimethyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

Aqueous NaOH (0.134 mL, 2.54 mmol, 50% in water) was added to a solution of Example 13C (120 mg, 0.214 mmol) in dioxane (4.2 mL) and water (1.4 mL). The reaction mixture was then heated at 90° C. for 1 hour. After cooling to room temperature the reaction mixture was acidified with 1M HCl and extracted three times with ethyl acetate. The combined organic phases were washed with brine, dried with MgSO₄, filtered, and concentrated under reduced pressure. The resulting residue was taken up in N,N-dimethylformamide (6 mL), cooled in an ice bath, and treated with HATU (98 mg, 0.257 mmol) and N,N-diisopropylethylamine (0.056 mL, 0.32 mmol). After 30 minutes cyclopropanamine (17.72 mg, 0.310 mmol) was added, and then after 15 minutes the reaction mixture was warmed to room temperature and stirred for an additional 1 hour. The reaction mixture was diluted with ethyl acetate, washed with saturated NaHCO₃ and brine, dried with Na₂SO₄, filtered, and concentrated under reduced pressure. The crude residue was purified by reverse phase HPLC (Waters)(Bridge 21×250 mm column, gradient 25-55% acetonitrile/10 mM NH₄HCO₃, flow rate 25 mL/minute) to afford the title compound (66.3mg, 0.153 mmol, 71.5% yield). ¹H NMR (400 MHz, CDCl₃) δ 11.91 (s, 1H), 7.87 (s, 1H), 6.89 (s, 1H), 6.71 (d, J=5.9 Hz, 2H), 6.49 (d, J=7.6 Hz, 1H), 6.10 (d, J=7.6 Hz, 1H), 3.74 (s, 3H), 3.34 (s, 3H), 3.13 (t, J=8.3 Hz, 1H), 2.78 (s, 1H), 2.29 (s, 3H), 2.18 (dd, J=8.9, 4.4 Hz, 1H), 1.88 (dd, J=7.7, 4.6 Hz, 1H), 0.86 (d, J=5.9 Hz, 2H), 0.62 (s, 2H).

### Example 14

### rac-N-cyclopropyl-4-[(1R,2S)-6′-methoxy-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

### Example 14A

### 6-methoxy-1-methylindoline-2,3-dione

A solution of 6-methoxyindoline-2,3-dione (1 g, 5.64 mmol, SCRC) in anhydrous N,N-dimethylformamide (40 mL) was cooled to 0° C., whereupon NaH (60% dispersion in mineral oil, 0.264 g, 6.60 mmol) was added in one portion and the reaction was stirred for 5 minutes. Iodomethane (0.413 mL, 6.60 mmol) was added and the reaction was stirred at 0° C. for 30 minutes. The mixture was then poured into saturated aqueous NH₄Cl and extracted with ethyl acetate. The combined organic portions were washed with water and brine, dried (MgSO₄), filtered, and concentrated to give the crude product (1.079 g), which was used without further purification.

### Example 14B

### 3-diazo-6-methoxy-1-methylindolin-2-one

To a stirring suspension of 4-methylbenzenesulfonohydrazide (1.108 g, 5.96 mmol, SCRC) in methanol (30 mL) at room temperature was added Example 14A (1.079 g, 5.64 mmol) in one portion. All the material dissolved after the addition and a yellow precipitate began to form within 10 minutes. After 3 hours the reaction was concentrated to a yellow solid. To this were added N-benzyl-N,N-diethylethanaminium chloride (0.026 g, 0.116 mmol, SCRC), dichloromethane (12 mL) and aqueous NaOH (12 mL, 4.00 mmol, 1M in water). The resulting mixture was stirred vigorously and heated at 40° C. for 15 hours, then the reaction was diluted with ethyl acetate and water and the layers were separated. The organic phase was washed with water and brine, dried with MgSO₄, filtered, and concentrated under reduced pressure. The residue was then purified via flash chromatography (ISCO Combiflash, 0-100% ethyl acetate/heptanes, 40 g Redisep silica column) to yield the title compound (0.412 g, 2.028 mmol, 35% yield) as a solid.

### Example 14C

### rac-ethyl 4-[(1R,2S)-6′-methoxy-′-methyl-2′-oxo-1′,2′-dihydrospiro[cyclopropane-1,3′-indol]-2-yl]-6-methyl-1-(4-methylbenzene-1-sulfonyl)-7-oxo-6,7-dihydro-1H-pyrrolo[2,3-c]pyridine-2-carboxylate

A solution of Example 14B (120 mg, 0.591 mmol) in dichloromethane (5 mL) was added over 12 hours via syringe pump through a reflux condenser to a solution of Example 1G (236 mg, 0.591 mmol) and rhodium(II) acetate dimer (2.61 mg, 5.91 μmol) in dichloromethane (10 mL) at 45° C. After an additional 6 hours the reaction mixture was concentrated and the crude residue was purified via flash chromatography (ISCO Combiflash, 50-100% ethyl acetate/heptanes, 40 g Redisep silica column) to provide the title compound (120 mg, 0.208 mmol, 35.3% yield).

### Example 14D

### rac-N-cyclopropyl-4-[(1R,2S)-6′-methoxy-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

Aqueous NaOH (0.134 mL, 2.54 mmol, 50% in water) was added to a solution of Example 14C (120 mg, 0.208 mmol) in dioxane (4.2 mL) and water (1.4 mL). The reaction was then heated at 90° C. for 1 hour. After cooling to room temperature the reaction mixture was acidified with 1M HCl and extracted three times with ethyl acetate. The combined organic phases were washed with brine, dried with MgSO₄, filtered, and concentrated under reduced pressure. The resulting residue was taken up in N,N-dimethylformamide (6 mL), cooled in an ice bath, and treated with HATU (95 mg, 0.250 mmol) and N,N-diisopropylethylamine (0.055 mL, 0.313 mmol). After 30 minutes cyclopropanamine (17.85 mg, 0.313 mmol) was added, and then after 15 minutes the reaction mixture was warmed to room temperature and stirred for an additional 1 hour. The reaction mixture was diluted with ethyl acetate, washed with saturated NaHCO₃ and brine, dried with Na₂SO₄, filtered, and concentrated under reduced pressure. The crude residue was purified by reverse phase HPLC (Waters)(Bridge 21×250 mm column, gradient 25-55% acetonitrile/10 mM NH₄HCO₃, flow rate 25 mL/minute) to afford the title compound (45.2 mg, 0.105 mmol, 50.1% yield). ¹H NMR (400 MHz, CDCl₃) δ 11.98 (s, 1H), 7.94 (s, 1H), 6.89 (s, 1H), 6.73 (s, 1H), 6.47 (d, J=1.9 Hz, 1H), 6.19 (dd, J=8.3, 2.0 Hz, 1H), 6.09 (d, J=8.2 Hz, 1H), 3.73 (s, 6H), 3.33 (s, 3H), 3.09 (t, J=8.2 Hz, 1H), 2.79 (s, 1H), 2.16 (dd, J=8.9, 4.5 Hz, 1H), 1.85 (dd, J=7.6, 4.6 Hz, 1H), 0.85 (t, J=6.2 Hz, 2H), 0.62 (s, 2H).

### Example 15

### rac-N-cyclopropyl-6-methyl-4-[(1R,2S)-1′-methyl-2′-oxo-6′-(trifluoromethoxy)-1′,2′-dihydrospiro[cyclopropane-1,3′-indol]-2-yl]-7-oxo-6,7-dihydro-1H-pyrrolo[2,3-c]pyridine-2-carboxamide

### Example 15A

### 1-methyl-6-(trifluoromethoxy)indoline-2,3-dione

A solution of 6-(trifluoromethoxy)indoline-2,3-dione (1 g, 4.33 mmol, SCRC) in anhydrous N,N-dimethylformamide (20 mL) was cooled to 0° C., whereupon NaH (60% dispersion in mineral oil, 0.202 g, 5.06 mmol) was added in one portion and the reaction was stirred for 5 minutes. Iodomethane (0.317 mL, 5.06 mmol) was added and the reaction was stirred at 0° C. for 30 minutes. The mixture was then poured into saturated aqueous NH₄Cl and extracted with ethyl acetate. The combined organic portions were washed with water and brine, dried (MgSO₄), filtered, and concentrated to give the crude product (1.061 g), which was used without further purification.

### Example 15B

### 3-diazo-1-methyl-6-(trifluoromethoxy)indolin-2-one

To a stirring suspension of 4-methylbenzenesulfonohydrazide (0.806 g, 4.33 mmol, SCRC) in methanol (30 mL) at room temperature was added Example 15A (1.061 g, 4.33 mmol) in one portion. All the material dissolved after the addition and a yellow precipitate began to form within 10 minutes. After 3 hours the reaction mixture was concentrated to a yellow solid. To this were added N-benzyl-N,N-diethylethanaminium chloride (0.020 g, 0.087 mmol, SCRC), dichloromethane (12 mL) and aqueous NaOH (12 mL, 4.00 mmol, 1M in water). The resulting mixture was stirred vigorously and heated at 40° C. for 15 hours, then the reaction was diluted with ethyl acetate and water and the layers were separated. The organic phase was washed with water and brine, dried with MgSO₄, filtered, and concentrated under reduced pressure. The residue was then purified via flash chromatography (ISCO Combiflash, 0-100% ethyl acetate/heptanes, 40 g Redisep silica column) to yield the title compound (0.390 g, 1.515 mmol, 35% yield) as a solid.

### Example 15C

### rac-ethyl 6-methyl-1-(4-methylbenzene-1-sulfonyl)-4-[(1R,2S)-1′-methyl-2′-oxo-6′-(trifluoromethoxy)-1′,2′-dihydrospiro[cyclopropane-1,3′-indol]-2-yl]-7-oxo-6,7-dihydro-1H-pyrrolo[2,3-c]pyridine-2-carboxylate

A solution of Example 15B (121 mg, 0.469 mmol) in dichloromethane (5 mL) was added over 12 hours via syringe pump through a reflux condenser to a solution of Example 1G (188 mg, 0.469 mmol) and rhodium(II) acetate dimer (2.075 mg, 4.69 μmol) in dichloromethane (10 mL) at 45° C. After an additional 6 hours the reaction mixture was concentrated and the crude residue was purified via flash chromatography (ISCO Combiflash, 50-100% ethyl acetate/heptanes, 40 g Redisep silica column) to provide the title compound (200 mg, 0.318 mmol, 67.7% yield).

### Example 15D

### rac-N-cyclopropyl-6-methyl-4-[(1R,2S)-1′-methyl-2′-oxo-6′-(trifluoromethoxy)-1′,2′-dihydrospiro[cyclopropane-1,3′-indol]-2-yl]-7-oxo-6,7-dihydro-1H-pyrrolo[2,3-c]pyridine-2-carboxamide

Aqueous NaOH (0.134 mL, 2.54 mmol, 50% in water) was added to a solution of Example 15C (200mg, 0.318 mmol) in dioxane (4.2 mL) and water (1.4 mL). The reaction was then heated at 90° C. for 1 hour. After cooling to room temperature the reaction mixture was acidified with 1M HCl and extracted three times with ethyl acetate. The combined organic phases were washed with brine, dried with MgSO₄, filtered, and concentrated under reduced pressure. The resulting residue was taken up in N,N-dimethylformamide (6 mL), cooled in an ice bath, and treated with HATU (145 mg, 0.381 mmol) and N,N-diisopropylethylamine (0.083 mL, 0.476 mmol). After 30 minutes cyclopropanamine (27.2 mg, 0.476 mmol) was added, and then after 15 minutes the reaction mixture was warmed to room temperature and stirred for an additional 1 hour. The reaction mixture was diluted with ethyl acetate, washed with saturated NaHCO₃ and brine, dried with Na₂SO₄, filtered, and concentrated under reduced pressure. The crude residue was purified by reverse phase HPLC (Waters)(Bridge 21×250 mm column, gradient 25-55% acetonitrile/10 mM NH₄HCO₃, flow rate 25 mL/minute) to afford the title compound (15.7mg, 0.031 mmol, 9.81% yield). ¹H NMR (400 MHz, CDCl₃) δ 7.65 (s, 1H), 6.92 (s, 1H), 6.75 (s, 1H), 6.63 (s, 1H), 6.54 (s, 1H), 6.20 (d, J=8.2 Hz, 1H), 3.74 (s, 3H), 3.37 (s, 3H), 3.19 (d, J=8.2 Hz, 1H), 2.79 (s, 1H), 2.25 (dd, J=8.9, 4.6 Hz, 1H), 1.95 (dd, J=7.7, 4.7 Hz, 1H), 0.87 (d, J=6.2 Hz, 2H), 0.63 (s, 2H).

### Example 16

### rac-N-cyclopropyl-4-[(1R,2S)-5′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

### Example 16A

### 6-methyl-7-oxo-4-vinyl-6,7-dihydro-1H-pyrrolo[2,3-c]pyridine-2-carboxylic acid

To a solution of Example 1G (4.20 g, 10.5 mmol) in dioxane (40 mL) was added 1M sodium hydroxide (31.5 mL, 31.5 mmol). The reaction mixture was heated at 70° C. for 2 hours, cooled, adjusted to pH 3 by addition of 1M HCl. The precipitate was collected by filtration and washed with water and dried to give the title compound (2.25 g, 98%).

### Example 16B

### N-cyclopropyl-6-methyl-7-oxo-4-vinyl-6,7-dihydro-1H-pyrrolo[2,3-c]pyridine-2-carboxamide

Example 16A (2.01 g, 9.20 mmol), HATU (3.85 g, 10.1 mmol) and triethylamine (3.85 mL, 27.6 mmol) were combined in dimethyl sulfoxide (20 mL). The reaction mixture was stirred at room temperature for 5 minutes, then cyclopropylamine was added (0.713 mL, 10.12 mmol) and the reaction mixture was stirred at room temperature for 20 hours. The mixture was partitioned with ethyl acetate and water. The organic layer was washed with saturated aqueous sodium chloride, dried with anhydrous sodium sulfate, filtered and concentrated. The residue was triturated with 10% ethyl acetate in heptanes to give the title compound (1.79 g, 76%).

### Example 16C

### N-(5-fluoro-2-oxoindolin-3-ylidene)-4-methylbenzenesulfonohydrazide

A mixture of 5-fluoroindoline-2,3-dione (1.65 g, 10.0 mmol) and 4-methylbenzenesulfonohydrazide (1.86 g, 10.0 mmol) in methanol (20 mL) was stirred at 60° C. for 2 hours, then cooled. The solid was collected via filtration and washed with methanol and dried to give the title compound (3.15 g, 94%).

### Example 16D

### 3-diazo-5-fluoroindolin-2-one

Example 16C (3.13 g, 9.40 mmol) and 1M sodium hydroxide in water (18.8 mL, 18.8 mmol) were combined in tetrahydrofuran (10 mL). The reaction mixture was stirred at 50° C. for 2 hours, cooled, carefully neutralized with excess dry ice, warmed to room temperature. The solid was collected via filtration and washed with water and dried to give the title compound (1.61 g, 97%).

### Example 16E

### 3-diazo-5-fluoro-1-methylindolin-2-one

Example 16D (1.60 g, 9.03 mmol), iodomethane (0.734 mL, 11.7 mmol) and potassium carbonate (1.87 g, 13.5 mmol) were combined in N,N-dimethylformamide (6 mL). The reaction mixture was stirred at room temperature for 20 hours, diluted with water, and stirred for 5 minutes. The solid was collected via filtration, washed with water and dried to provide the title compound (1.56 g, 90%).

### Example 16F

### rac-N-cyclopropyl-4-[(1R,2S)-5′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

Example 16B (129 mg, 0.500 mmol), Example 16E (143 mg, 0.750 mmol) and rhodium(II) acetate dimer (2.2 mg, 5.0 μmol) were combined in tetrahydrofuran (10 mL). The reaction mixture was heated at 45° C. for 16 hours. Additional Example 16E (48 mg, 0.25 mmol) was added and heating was continued at 45° C. for another 24 hours. The reaction mixture was cooled and partitioned with ethyl acetate and water. The aqueous layer was extracted with ethyl acetate. The combined organic ethyl acetate layers were washed with saturated aqueous sodium chloride, dried with anhydrous sodium sulfate, treated with 3-mercaptopropyl functionalized silica gel, filtered, and concentrated. The residue was purified by flash chromatography (silica gel, 2-6% methanol in dichloromethane) to give the title compound (138 mg, 66%).¹H NMR (500 MHz, DMSO-d₆) δ 12.13 (s, 1H), 8.31 (d, J=4.1 Hz, 1H), 7.44 (d, J=1.2 Hz, 1H), 7.02-6.92 (m, 2H), 6.43 (s, 1H), 6.26 (dd, J=8.8, 2.6 Hz, 1H), 3.57 (s, 3H), 3.26 (s, 3H), 2.99-2.94 (m, 1H), 2.76-2.71 (m, 1H), 2.35 (dd, J=7.9, 4.7 Hz, 1H), 2.02 (dd, J=9.0, 4.7 Hz, 1H), 0.70-0.62 (m, 2H), 0.51-0.44 (m, 2H). (ESI+) m/z 421 (M+H)⁺.

### Example 17

### N-cyclopropyl-4-[(1S,2R)-5′-fluoro-′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

The product from Example 16F was purified by chiral chromatography on ChiralPak IC column eluting with 40% methanol in carbon dioxide. The title compound was the second-eluting enantiomer. ¹H NMR (400 MHz, DMSO-d₆) δ 12.09 (s, 1H), 8.27 (d, J=4.1 Hz, 1H), 7.41 (d, J=1.2 Hz, 1H), 7.00-6.87 (m, 2H), 6.40 (s, 1H), 6.23 (dd, J=8.8, 2.6 Hz, 1H), 3.54 (s, 3H), 3.24 (s, 3H), 2.93 (t, J=8.4 Hz, 1H), 2.75-2.67 (m, 1H), 2.31 (dd, J=8.0, 4.8 Hz, 1H), 2.00 (dd, J=9.0, 4.7 Hz, 1H), 0.68-0.57 (m, 2H), 0.50-0.38 (m, 2H). (ESI+) m/z 421 (M+H)⁺.

### Example 18

### N-cyclopropyl-4-[(1R,2S)-5′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

The product from Example 16F was purified by chiral chromatography on ChiralPak IC column eluting with 40% methanol in carbon dioxide. The title compound was the first-eluting enantiomer. ¹H NMR (400 MHz, DMSO-d₆) δ 12.09 (s, 1H), 8.27 (d, J=4.1 Hz, 1H), 7.41 (d, J=1.3 Hz, 1H), 7.00-6.86 (m, 2H), 6.40 (s, 1H), 6.24 (dd, J=8.8, 2.6 Hz, 1H), 3.54 (s, 3H), 3.24 (s, 3H), 2.93 (t, J=8.3 Hz, 1H), 2.76-2.66 (m, 1H), 2.31 (dd, J=8.0, 4.8 Hz, 1H), 2.00 (dd, J=9.0, 4.7 Hz, 1H), 0.68-0.57 (m, 2H), 0.50-0.39 (m, 2H). (ESI+) m/z 421 (M+H)⁺.

### Example 19

### rac-N-cyclopropyl-6-methyl-7-oxo-4-[(1R,2S)-2′-oxo-1′,2′-dihydrospiro[cyclopropane-1,3′-indol]-2-yl]-6,7-dihydro-1H-pyrrolo[2,3-c]pyridine-2-carboxamide

A solution of 3-diazoindolin-2-one (95 mg, 0.60 mmol) in dichloromethane (2 mL) was added dropwise to the mixture of Example 16B (129 mg, 0.500 mmol) and rhodium (II) acetate dimer (2.2 mg, 5.0 μmol) in dichloromethane (8 mL). The reaction mixture was heated at 45° C. for 3 hours, cooled and concentrated. The residue was purified by flash chromatography (silica gel, 2-6% methanol in dichloromethane) to give the title compound (64 mg, 33%). ¹H NMR (400 MHz, DMSO-d₆) δ 12.05 (s, br, 1H), 10.56 (s, 1H), 8.30 (d, J=4.2 Hz, 1H), 7.40 (d, J=1.3 Hz, 1H), 7.00-6.93 (m, 1H), 6.81 (d, J=7.7 Hz, 1H), 6.62-6.56 (m, 1H), 6.45 (s, 1H), 6.36 (d, J=7.3 Hz, 1H), 3.55 (s, 3H), 2.88-2.79 (m, 1H), 2.78-2.68 (m, 1H), 2.19 (dd, J=7.9, 4.6 Hz, 1H), 1.92 (dd, J=8.9, 4.5 Hz, 1H), 0.68-0.61 (m, 2H), 0.51-0.42 (m, 2H). (ESI+) m/z 389 (M+H)⁺.

### Example 20

### N-cyclopropyl-6-methyl-7-oxo-4-[(1S,2R)-2′-oxo-1′,2′-dihydrospiro[cyclopropane-1,3′-indol]-2-yl]-6,7-dihydro-1H-pyrrolo[2,3-c]pyridine-2-carboxamide

The product from Example 19 was purified by chiral chromatography on a YMC-SA column eluting with 10-51% of 1:1 mixture of methanol and ethanol in carbon dioxide. The title compound was the first-eluting enantiomer. ¹H NMR (500 MHz, DMSO-d₆) δ 12.03 (s, br, 1H), 10.56 (s, 1H), 8.31 (d, J=4.1 Hz, 1H), 7.40 (s, 1H), 7.00-6.95 (m, 1H), 6.81 (d, J=7.7 Hz, 1H), 6.62-6.56 (m, 1H), 6.44 (s, 1H), 6.36 (d, J=7.5 Hz, 1H), 3.55 (s, 3H), 2.86-2.82 (m, 1H), 2.77-2.68 (m, 1H), 2.19 (dd, J=7.9, 4.6 Hz, 1H), 1.92 (dd, J=8.9, 4.6 Hz, 1H), 0.68-0.62 (m, 2H), 0.50-0.44 (m, 2H). (ESI+) m/z 389 (M+H)⁺.

### Example 21

### N-cyclopropyl-6-methyl-7-oxo-4-[(1R,2S)-2′-oxo-1′,2′-dihydrospiro[cyclopropane-1,3′-indol]-2-yl]-6,7-dihydro-1H-pyrrolo[2,3-c]pyridine-2-carboxamide

The product from Example 19 was purified by chiral chromatography on a YMC-SA column eluting with 10-51% of 1:1 mixture of methanol and ethanol in carbon dioxide. The title compound was the second-eluting enantiomer. ¹H NMR (400 MHz, DMSO-d₆) δ 12.01 (s, br, 1H), 10.55 (s, 1H), 8.28 (d, J=4.1 Hz, 1H), 7.38 (s, 1H), 6.99-6.92 (m, 1H), 6.79 (d, J=7.7 Hz, 1H), 6.62-6.55 (m, 1H), 6.42 (s, 1H), 6.34 (d, J=7.4 Hz, 1H), 3.53 (s, 3H), 2.82 (t, J=8.3 Hz, 1H), 2.76-2.65 (m, 1H), 2.18 (dd, J=7.8, 4.6 Hz, 1H), 1.90 (dd, J=8.9, 4.6 Hz, 1H), 0.69-0.59 (m, 2H), 0.51-0.40 (m, 2H). (ESI+) m/z 389 (M+H)⁺.

### Example 22

### rac-N-cyclopropyl-4-[(1R,2S)-1′-ethyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

### Example 22A

### 1-ethylindoline-2,3-dione

A mixture of indoline-2,3-dione (0.5 g, 3.40 mmol, Aldrich), potassium carbonate (0.705 g, 5.10 mmol), and iodoethane (0.412 mL, 5.10 mmol) in N,N-dimethylformamide (6.80 mL) was stirred at room temperature. After 22 hours the reaction was poured into 75 mL of water and extracted with ethyl acetate. The organic phase was washed with water and brine, dried with MgSO₄, filtered, and concentrated under reduced pressure to yield the title compound (505 mg, 2.88 mmol, 85% yield) as a solid. ¹H NMR (400 MHz, DMSO-d₆) δ 7.65 (td, J=7.8, 1.4 Hz, 1H), 7.53 (dd, J=7.4, 1.3 Hz, 1H), 7.18 (d, J=8.0 Hz, 1H), 7.11 (td, J=7.5, 0.9 Hz, 1H), 3.69 (q, J=7.2 Hz, 2H), 1.17 (t, J=7.2 Hz, 3H).

### Example 22B

### 3-diazo-1-ethylindolin-2-one

A solution of Example 22A (0.505 g, 2.88 mmol) and 4-methylbenzenesulfonohydrazide (0.537 g, 2.88 mmol, Aldrich) in methanol (12 mL) was stirred at room temperature. After 2 hours the resulting mixture was concentrated to a yellow solid and tetrahydrofuran (12 mL) was added. A NaOH solution (28.8 mL, 5.77 mmol, 0.2 M in water) was then added dropwise via addition funnel. After stirring for 18 hours the reaction was diluted with ethyl acetate and water. The layers were separated and the organic phase was washed with brine, dried with MgSO₄, filtered, and concentrated under reduced pressure. The crude residue was then purified via flash chromatography (ISCO Combiflash, 0-50% ethyl acetate/heptanes, 40 g Redisep silica column) to yield the title compound (273 mg, 1.458 mmol, 50.6% yield) as oil. ¹H NMR (501 MHz, CDCl₃) δ 7.26-7.19 (m, 2H), 7.11 (td, J=7.6, 1.0 Hz, 1H), 6.97 (dt, J=7.8, 0.9 Hz, 1H), 3.92 (q, J=7.2 Hz, 2H), 1.33 (t, J=7.2 Hz, 3H).

### Example 22C

### rac-ethyl 4-[(1R,2S)-1′-ethyl-2′-oxo-1′,2′-dihydrospiro[cyclopropane-1,3′-indol]-2-yl]-6-methyl-1-(4-methylbenzene-1-sulfonyl)-7-oxo-6,7-dihydro-1H-pyrrolo [2,3-c]pyridine-2-carboxylate

A solution of Example 22B (45 mg, 0.240 mmol) in dichloromethane (1 mL) was added over 1 hour via syringe pump to a solution of Example 1G (74.0 mg, 0.185 mmol) and rhodium(II) acetate dimer (0.409 mg, 0.925 μmol) in dichloromethane (2 mL) at room temperature. After stirring for 18 hours the reaction mixture was concentrated under reduced pressure and the crude residue was purified via flash chromatography (ISCO Combiflash, 50-100% ethyl acetate/heptanes, 40 g Redisep silica column) to yield the title compound (87 mg, 0.155 mmol, 84% yield) as solid. ¹H NMR (501 MHz, CDCl₃) δ 8.52-8.43 (m, 2H), 7.44-7.37 (m, 2H), 7.20 (td, J=7.8, 1.2 Hz, 1H), 7.00 (d, J=1.4 Hz, 1H), 6.94 (d, J=7.8 Hz, 1H), 6.75 (td, J=7.6, 1.0 Hz, 1H), 6.48 (s, 1H), 6.26-6.19 (m, 1H), 4.40 (q, J=7.1 Hz, 2H), 3.94 (ddp, J=21.3, 14.3, 7.2 Hz, 2H), 3.60 (s, 3H), 3.03 (ddd, J=9.1, 7.7, 1.4 Hz, 1H), 2.46 (s, 3H), 2.25 (dd, J=9.0, 4.6 Hz, 1H), 1.83 (dd, J=7.7, 4.6 Hz, 1H), 1.38 (q, J=7.2 Hz, 6H); MS (ESI+) m/z 560.1 (M+H)⁺.

### Example 22D

### rac-N-cyclopropyl-4-[(1R,2S)-1′-ethyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

A mixture of Example 22C (86 mg, 0.154 mmol) and NaOH (0.461 mL, 0.461 mmol, 1M aqueous solution) in dioxane (1.5 mL) was heated to 80° C. After 1 hour the reaction mixture was cooled to room temperature and acidified with 15 mL of 1M HCl. The resulting precipitate was collected by filtration and washed with water. N,N-dimethylformamide was added to the residue and the mixture was cooled in an ice bath before HATU (70.1 mg, 0.184 mmol) and N,N-diisopropylethylamine (0.067 mL, 0.384 mmol) were added. After 20 minutes cyclopropylamine (21.93 mg, 0.384 mmol) was added in one portion and the reaction mixture was allowed to warm to room temperature. After 18 hours the solution was diluted with ethyl acetate and washed with a saturated aqueous NaHCO₃ solution and brine. The organic phase was dried with MgSO₄, filtered, and concentrated under reduced pressure. The crude residue was then purified via reverse-phase HPLC (5-95% acetonitrile/water with 0.1% trifluoroacetic acid) to yield the title compound (11.9 mg, 0.029 mmol, 18.59% yield) as solid. ¹H NMR (400 MHz, DMSO-d₆) δ 12.03 (s, 1H), 8.24 (d, J=4.2 Hz, 1H), 7.40 (d, J=1.3 Hz, 1H), 7.10-6.93 (m, 2H), 6.64 (td, J=7.5, 1.3 Hz, 1H), 6.42-6.38 (m, 1H), 6.37-6.34 (m, 1H), 3.81 (q, J=7.1 Hz, 2H), 3.53 (s, 3H), 2.87 (ddd, J=9.1, 7.9, 1.3 Hz, 1H), 2.70 (tq, J=7.7, 3.9 Hz, 1H), 2.23 (dd, J=7.9, 4.7 Hz, 1H), 1.98 (dd, J=8.9, 4.7 Hz, 1H), 1.18 (t, J=7.2 Hz, 3H), 0.63 (td, J=7.0, 4.8 Hz, 2H), 0.46-0.38 (m, 2H).MS (ESI−) m/z 415.1 (M−H)⁻.

### Example 23

### rac-N-cyclopropyl-6-methyl-7-oxo-4-[(1R,2S)-2′-oxo-′-(2,2,2-trifluoroethyl)-1′,2′-dihydrospiro[cyclopropane-1,3′-indol]-2-yl]-6,7-dihydro-1H-pyrrolo[2,3-c]pyridine-2-carboxamide

### Example 23A

### 1-(2,2,2-trifluoroethyl)indoline-2,3-dione

A suspension of NaH (0.598 g, 14.95 mmol, 60% suspension in mineral oil) in N,N-dimethylformamide (20 mL) was cooled in an ice bath. A solution of indoline-2,3-dione (2 g, 13.59 mmol, Aldrich) in N,N-dimethylformamide (7 mL) was then added dropwise. After 30 minutes 1,1,1-trifluoro-2-iodoethane (1.541 mL, 15.63 mmol) was added and the reaction was heated to 80° C. After 20 hours the reaction mixture was poured into a saturated aqueous NH₄Cl solution and extracted with ethyl acetate. The organic phase was washed with water and brine, dried with MgSO₄, filtered, and concentrated. The crude residue was then purified via flash chromatography (ISCO Combiflash, 0-75% ethyl acetate/heptanes 80 g Redisep silica column) to yield the title compound (722 mg, 3.15 mmol, 23.18% yield) as solid. ¹H NMR (400 MHz, DMSO-d₆) δ 7.69 (td, J=7.8, 1.4 Hz, 1H), 7.58 (ddd, J=7.5, 1.4, 0.6 Hz, 1H), 7.29 (dq, J=8.2, 0.8 Hz, 1H), 7.17 (td, J=7.5, 0.8 Hz, 1H), 4.61 (q, J=9.4 Hz, 2H).

### Example 23B

### 3-diazo-1-(2,2,2-trifluoroethyl)indolin-2-one

Example 23A (0.722 g, 3.15 mmol) and 4-methylbenzenesulfonohydrazide (0.587 g, 3.15 mmol, Aldrich) were combined in methanol (16 mL) and stirred at room temperature. After 2 hours the resulting mixture was concentrated. A NaOH solution (8 mL, 8.00 mmol, 1M in water) and dichloromethane (8 mL) were added to the resulting residue and the mixture was heated to 40° C. After 18 hours the reaction was diluted with ethyl acetate and washed with water and brine. The organic phase was dried with MgSO₄, filtered, and concentrated. The crude residue was then purified via flash chromatography (ISCO Combiflash, 0-100% ethyl acetate/heptanes, 40 g Redisep silica column) to yield the title compound (157 mg, 0.651 mmol, 20.66% yield) as solid. ¹H NMR (501 MHz, CDCl₃) δ 7.29-7.24 (m, 2H), 7.21-7.16 (m, 1H), 7.08-7.03 (m, 1H), 4.45 (q, J=8.6 Hz, 2H).

### Example 23C

### rac-ethyl 6-methyl-1-(4-methylbenzene-1-sulfonyl)-7-oxo-4-[(1R,SR)-2′-oxo-1′-(2,2,2-trifluoroethyl)-1′,2′-dihydrospiro[cyclopropane-1,3′-indol]-2-yl]-6,7-dihydro-1H-pyrrolo[2,3-c]pyridine-2-carboxylate

A solution of Example 23B (60 mg, 0.249 mmol) in dichloromethane (2 mL) was added over 12 hours via syringe pump to a solution of Example 1G (77 mg, 0.191 mmol) and rhodium(II) acetate dimer (0.846 mg, 1.914 μmol) in dichloromethane (4 mL) at 45° C. After 18 hours the reaction mixture was concentrated and the crude residue was purified via flash chromatography (ISCO Combiflash, 50-100% ethyl acetate/heptanes, 40 g Redisep silica column) to yield the title compound (114 mg, 0.186 mmol, 97% yield) as solid. ¹H NMR (400 MHz, CDCl₃) δ 8.52-8.45 (m, 2H), 7.40-7.36 (m, 2H), 7.20 (td, J=7.8, 1.2 Hz, 1H), 7.04 (d, J=1.4 Hz, 1H), 6.98 (d, J=7.9 Hz, 1H), 6.79 (td, J=7.6, 1.0 Hz, 1H), 6.39 (s, 1H), 6.27-6.20 (m, 1H), 4.63 (dq, J=15.3, 8.9 Hz, 1H), 4.45-4.23 (m, 3H), 3.08 (ddd, J=9.2, 7.8, 1.5 Hz, 1H), 2.44 (s, 3H), 2.32 (dd, J=9.0, 4.7 Hz, 1H), 1.91 (dd, J=7.8, 4.8 Hz, 1H); MS (ESI+) m/z 614.0 (M+H)⁺.

### Example 23D

### rac-N-cyclopropyl-6-methyl-7-oxo-4-[(1R,2S)-2′-oxo-′-(2,2,2-trifluoroethyl)-1′,2′-dihydrospiro[cyclopropane-1,3′-indol]-2-yl]-6,7-dihydro-1H-pyrrolo[2,3-c]pyridine-2-carboxamide

Aqueous NaOH (0.557 mL, 0.557 mmol, 1M in water) was added to a solution of Example 23C (114 mg, 0.186 mmol) in dioxane (1 mL) at room temperature. The reaction mixture was then heated to 80° C. After 90 minutes the reaction mixture was acidified with 15 mL of 1M HCl and extracted three times with ethyl acetate. The combined organic phases were dried with MgSO₄, filtered, and concentrated under reduced pressure. The resulting residue was taken up in N,N-dimethylformamide (1.3 mL) and cooled in an ice bath. HATU (85 mg, 0.223 mmol) and N,N-diisopropylethylamine (0.049 mL, 0.279 mmol) were added sequentially. After 20 minutes cyclopropylamine (15.91 mg, 0.279 mmol) was added in one portion and the reaction mixture was allowed to warm to room temperature. After an additional 1 hour the reaction mixture was diluted with ethyl acetate and washed twice with a saturated aqueous NaHCO₃ solution and once with brine. The organic phase was dried with MgSO₄, filtered, and concentrated under reduced pressure. The crude residue was purified via reverse phase HPLC (5-95% acetonitrile/water with 0.5% trifluoroacetic acid) to yield the title compound (21.6 mg, 0.046 mmol, 24.71% yield) as solid. ¹H NMR (501 MHz, DMSO-d₆) δ 12.08 (d, J=2.3 Hz, 1H), 8.22 (d, J=4.1 Hz, 1H), 7.45 (d, J=1.3 Hz, 1H), 7.33-7.04 (m, 3H), 6.73 (td, J=7.5, 1.1 Hz, 1H), 6.46 (dd, J=7.6, 1.2 Hz, 1H), 6.35 (d, J=2.2 Hz, 1H), 4.75 (qd, J=9.3, 5.9 Hz, 2H), 3.56 (s, 3H), 3.02-2.93 (m, 1H), 2.72 (tp, J=7.6, 3.9 Hz, 1H), 2.39-2.32 (m, 1H), 2.09 (dd, J=9.0, 4.8 Hz, 1H), 0.68-0.59 (m, 2H), 0.44 (dt, J=7.0, 4.3 Hz, 2H); MS (ESI+) m/z 471.0 (M+H)⁺.

### Example 24

### N-cyclopropyl-6-methyl-7-oxo-4-[(1S,2R)-2′-oxo-′-(2,2,2-trifluoroethyl)-1′,2′-dihydrospiro[cyclopropane-1,3′-indol]-2-yl]-6,7-dihydro-1H-pyrrolo[2,3-c]pyridine-2-carboxamide

The enantiomers of Example 23D (103 mg) were separated by chiral HPLC (Waters Instrument, Daicel IC column (20×250 mm), 50 heptane with 0.1% diethylamine/ethanol at 20 mL/minute) to yield the title compound (45 mg, 0.096 mmol, 43.7% yield) with a retention time of 14.0 minutes. ¹H NMR (501 MHz, DMSO-d₆) δ 12.08 (d, J=2.3 Hz, 1H), 8.22 (d, J=4.1 Hz, 1H), 7.45 (d, J=1.3 Hz, 1H), 7.33-7.04 (m, 3H), 6.73 (td, J=7.5, 1.1 Hz, 1H), 6.46 (dd, J=7.6, 1.2 Hz, 1H), 6.35 (d, J=2.2 Hz, 1H), 4.75 (qd, J=9.3, 5.9 Hz, 2H), 3.56 (s, 3H), 3.02-2.93 (m, 1H), 2.72 (tp, J=7.6, 3.9 Hz, 1H), 2.39-2.32 (m, 1H), 2.09 (dd, J=9.0, 4.8 Hz, 1H), 0.68-0.59 (m, 2H), 0.44 (dt, J=7.0, 4.3 Hz, 2H); MS (ESI+) m/z 471.0 (M+H)⁺.

### Example 25

### N-cyclopropyl-6-methyl-7-oxo-4-[(1R,2S)-2′-oxo-′-(2,2,2-trifluoroethyl)-1′,2′-dihydrospiro[cyclopropane-1,3′-indol]-2-yl]-6,7-dihydro-1H-pyrrolo[2,3-c]pyridine-2-carboxamide

Following the purification condition as described in Example 24 the title compound (47 mg, 0.100 mmol, 45.6% yield) was isolated as solid with a retention time of 7.9 minutes. ¹H NMR (501 MHz, DMSO-d₆) δ 12.08 (d, J=2.3 Hz, 1H), 8.22 (d, J=4.1 Hz, 1H), 7.45 (d, J=1.3 Hz, 1H), 7.33-7.04 (m, 3H), 6.73 (td, J=7.5, 1.1 Hz, 1H), 6.46 (dd, J=7.6, 1.2 Hz, 1H), 6.35 (d, J=2.2 Hz, 1H), 4.75 (qd, J=9.3, 5.9 Hz, 2H), 3.56 (s, 3H), 3.02-2.93 (m, 1H), 2.72 (tp, J=7.6, 3.9 Hz, 1H), 2.39-2.32 (m, 1H), 2.09 (dd, J=9.0, 4.8 Hz, 1H), 0.68-0.59 (m, 2H), 0.44 (dt, J=7.0, 4.3 Hz, 2H); MS (ESI+) m/z 471.0 (M+H)⁺.

### Example 26

### rac-N-cyclopropyl-4-[(1R,2S)-1′-cyclopropyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

### Example 26A

### 1-cyclopropylindoline-2,3-dione

A suspension of copper (II) acetate (545 mg, 3.00 mmol) and 2,2′-bipyridine (469 mg, 3.00 mmol) in 1,2-dichloroethane (20 mL) was heated at 70° C. for 5 minutes. This mixture was then poured into a stirring suspension of indoline-2,3-dione (441 mg, 3 mmol), cyclopropylboronic acid (515 mg, 6.00 mmol), and sodium carbonate (636 mg, 6.00 mmol) in 1,2-dichloroethane (10 mL). The resulting mixture was heated to 70° C. and stirred open to air. After 2 days the reaction was cooled to room temperature and quenched with a saturated aqueous NH₄Cl solution and water. The resulting mixture was extracted three times with dichloromethane. The combined organic layers were washed with brine, dried with MgSO₄, filtered, and concentrated under reduced pressure. The crude residue was then purified via flash chromatography (ISCO Combiflash, dichloromethane, 40 g Redisep silica column) to yield the title compound (364 mg, 1.944 mmol, 64.8% yield) as solid. ¹H NMR (400 MHz, DMSO-d₆) δ 7.67 (td, J=7.8, 1.4 Hz, 1H), 7.51 (dd, J=7.3, 1.3 Hz, 1H), 7.22 (dd, J=7.9, 0.8 Hz, 1H), 7.12 (td, J=7.5, 0.9 Hz, 1H), 2.68 (tt, J=7.1, 3.8 Hz, 1H), 0.99 (ddd, J=7.2, 4.1, 3.1 Hz, 2H), 0.85-0.77 (m, 2H); MS (ESI+) m/z 188.0 (M+H)⁺.

### Example 26B

### 1-cyclopropyl-3-diazoindolin-2-one

A solution of Example 26A (0.147 g, 0.785 mmol) and 4-methylbenzenesulfonohydrazide (0.146 g, 0.785 mmol, Aldrich) in methanol (3 mL) was stirred at room temperature for 1 hour. The mixture was then concentrated and benzyltriethylammonium chloride (8.94 mg, 0.039 mmol), dichloromethane (2.4 mL) and NaOH (2.356 mL, 2.356 mmol, 1M in water) were added to the resulting residue. The reaction was heated at 45° C. for 15 hours. The reaction mixture was then diluted with ethyl acetate and washed twice with water and once with brine. The organic phase was dried with MgSO₄, filtered, and concentrated under reduced pressure. The crude residue was purified via flash chromatography (ISCO Combiflash, 0-50% ethyl acetate/heptanes, 40 g Redisep silica column) to yield the title compound (46.7 mg, 0.234 mmol, 29.9% yield) as solid. ¹H NMR (400 MHz, CDCl₃) δ 7.56-7.47 (m, 1H), 7.34-7.25 (m, 1H), 7.25-7.03 (m, 2H), 2.76 (dtt, J=14.3, 7.2, 3.8 Hz, 1H), 1.13 (td, J=7.4, 5.3 Hz, 2H), 1.03-0.94 (m, 2H).

### Example 26C

### rac-ethyl 4-[(1R,2S)-1′-cyclopropyl-2′-oxo-1′,2′-dihydrospiro[cyclopropane-1,3′-indol]-2-yl]-6-methyl-1-(4-methylbenzene-1-sulfonyl)-7-oxo-6,7-dihydro-1H-pyrrolo[2,3-c]pyridine-2-carboxylate

A solution of Example 26B (40 mg, 0.201 mmol) in dichloromethane (1 mL) was added over 3 hours via syringe pump to a solution of Example 1G (67.0 mg, 0.167 mmol) and rhodium(II) acetate dimer (0.370 mg, 0.837 μmol) in dichloromethane (2.5 mL) at room temperature. After 18 hours the reaction mixture was concentrated under reduced pressure and the crude residue was purified via flash chromatography (ISCO Combiflash, 50-100% ethyl acetate/heptanes, 12 g Redisep silica column to yield the title compound (90.2 mg, 0.158 mmol, 94% yield) as solid. ¹H NMR (400 MHz, CDCl₃) δ 8.48 (d, J=8.2 Hz, 2H), 7.40 (d, J=8.1 Hz, 2H), 7.26-7.14 (m, 2H), 6.97 (d, J=1.3 Hz, 1H), 6.76 (td, J=7.2, 1.8 Hz, 1H), 6.48 (d, J=0.9 Hz, 1H), 6.20 (d, J=7.5 Hz, 1H), 4.41 (q, J=7.1 Hz, 2H), 3.58 (s, 3H), 3.06-2.90 (m, 1H), 2.80 (tt, J=7.2, 3.8 Hz, 1H), 2.46 (s, 3H), 2.21 (dd, J=9.0, 4.5 Hz, 1H), 1.79 (dd, J=7.7, 4.6 Hz, 1H), 1.41 (dd, J=7.5, 6.7 Hz, 3H), 1.21-1.11 (m, 2H), 1.03 (p, J=4.0 Hz, 2H); MS (ESI+) m/z 572.3 (M+H)⁺.

### Example 26D

### rac-N-cyclopropyl-4-[(1R,2S)-1′-cyclopropyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

A mixture of Example 26C (50 mg, 0.087 mmol) and NaOH (0.262 mL, 0.262 mmol, 1M in water) in dioxane (0.75 mL) was heated to 80° C. After 90 minutes the reaction mixture was acidified with 4M HCl (0.3 mL) in dioxane and then concentrated under reduced pressure. The resulting residue was then taken up in N,N-dimethylformamide (1 mL) and cooled in an ice bath. N,N-diisopropylethylamine (0.076 mL, 0.437 mmol) and HATU (39.9 mg, 0.105 mmol) were then added sequentially. After 20 minutes cyclopropanamine (12.48 mg, 0.219 mmol) was added and the solution was allowed to warm to room temperature. After 18 hours the reaction mixture was diluted with ethyl acetate and washed with a saturated aqueous NaHCO₃ solution and brine. The organic phase was then dried with MgSO₄, filtered, and concentrated under reduced pressure. The crude residue was purified via reverse-phase HPLC (5-95% acetonitrile/water with 0.1% trifluoroacetic acid) to yield the title compound (3.5 mg, 8.17 μmol, 9.34% yield) as solid. ¹H NMR (501 MHz, DMSO-d₆) δ 12.04 (s, 1H), 8.26 (d, J=4.2 Hz, 1H), 7.39 (d, J=1.3 Hz, 1H), 7.10-7.06 (m, 2H), 6.67 (ddd, J=7.6, 5.5, 3.2 Hz, 1H), 6.43-6.28 (m, 2H), 3.54 (s, 3H), 2.87-2.82 (m, 1H), 2.75 (dtt, J=27.8, 7.2, 3.7 Hz, 2H), 2.21 (dd, J=7.9, 4.7 Hz, 1H), 1.94 (dd, J=8.9, 4.7 Hz, 1H), 1.07-1.00 (m, 2H), 0.88-0.80 (m, 2H), 0.65 (tt, J=6.9, 3.3 Hz, 2H), 0.49-0.41 (m, 2H); MS (ESI+) m/z 429.2 (M+H)⁺.

### Example 27

### rac-N-cyclopropyl-4-[(1R,2S)-1′-(2-methoxyethyl)-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

### Example 27A

### rac-ethyl 6-methyl-1-(4-methylbenzene-1-sulfonyl)-7-oxo-4-[(1R,2S)-2′-oxo-1′,2′-dihydrospiro[cyclopropane-1,3′-indol]-2-yl]-6,7-dihydro-1H-pyrrolo[2,3-c]pyridine-2-carboxylate

A vial containing Example 1G (110 mg, 0.275 mmol), 3-diazoindolin-2-one (54.6 mg, 0.343 mmol, prepared as described in J. Am. Chem. Soc., 2005, 127, 11505), and rhodium(II) acetate dimer (0.607 mg, 1.373 μmol) was evacuated and backfilled three times with nitrogen before being charged with dichloromethane (5.5 mL). The reaction mixture was heated at 50° C. for 2 hours. After cooling to room temperature the reaction mixture was concentrated and the resulting residue was purified via flash chromatography (ISCO Combiflash, 0-5% methanol/dichloromethane, 12 g Redisep Gold silica column) to yield the title compound (123 mg, 0.231 mmol, 84% yield) as solid. ¹H NMR (400 MHz, CDCl₃) δ 8.51-8.40 (m, 2H), 7.82 (s, 1H), 7.40 (d, J=8.2 Hz, 2H), 7.15 (td, J=7.7, 1.1 Hz, 1H), 7.02 (d, J=1.4 Hz, 1H), 6.94 (d, J=7.8 Hz, 1H), 6.75 (td, J=7.6, 1.0 Hz, 1H), 6.55 (s, 1H), 6.21 (d, J=7.5 Hz, 1H), 4.42 (qd, J=7.1, 1.6 Hz, 2H), 3.60 (s, 3H), 3.05 (t, J=8.2 Hz, 1H), 2.46 (s, 3H), 2.27 (dd, J=9.0, 4.6 Hz, 1H), 1.86 (dd, J=7.8, 4.6 Hz, 1H), 1.41 (t, J=7.1 Hz, 3H)MS (ESI+) m/z 532.1 (M+H)⁺.

### Example 27B

### rac-ethyl 4-[(1R,2S)-1′-(2-methoxyethyl)-2′-oxo-1′,2′-dihydrospiro[cyclopropane-1,3′-indol]-2-yl]-6-methyl-1-(4-methylbenzene-1-sulfonyl)-7-oxo-6,7-dihydro-1H-pyrrolo[2,3-c]pyridine-2-carboxylate

1-Bromo-2-methoxyethane (11.49 μL, 0.122 mmol, Aldrich) was added to a mixture of Example 27A (50 mg, 0.094 mmol), potassium carbonate (19.50 mg, 0.141 mmol), and tetrabutylammonium iodide (1.737 mg, 4.70 μmol) in N,N-dimethylformamide (376 μL) at room temperature. The reaction was heated at 80° C. for 18 hours. After cooling to room temperature the reaction mixture was diluted with ethyl acetate and washed with a saturated aqueous NaHCO₃ solution and brine. The organic phase was dried with MgSO₄, filtered, and concentrated under reduced pressure. The crude residue was purified via reverse phase HPLC (5-95% acetonitrile/water with 0.1% trifluoroacetic acid) to yield the title compound (16 mg, 0.027 mmol, 28.8% yield). ¹H NMR (400 MHz, CDCl₃) δ 8.54-8.37 (m, 2H), 7.42-7.32 (m, 2H), 7.15 (dtd, J=15.1, 7.8, 1.2 Hz, 1H), 7.04-6.94 (m, 1H), 6.77-6.58 (m, 1H), 6.52 (d, J=14.0 Hz, 1H), 6.32-6.14 (m, 1H), 5.35-5.15 (m, 1H), 4.49-4.22 (m, 2H), 4.18-3.89 (m, 2H), 3.78-3.63 (m, 2H), 3.59 (d, J=13.1 Hz, 3H), 3.39 (d, J=5.3 Hz, 3H), 3.25 (d, J=0.9 Hz, 1H), 3.06 (dddd, J=34.8, 9.2, 7.8, 1.5 Hz, 1H), 2.45 (s, 2H), 2.24 (dd, J=9.0, 4.5 Hz, 1H), 1.86 (ddd, J=33.1, 7.8, 4.6 Hz, 1H), 1.36 (dt, J=15.2, 7.1 Hz, 3H); MS (ESI+) m/z 590.2 (M+H)⁺.

### Example 27C

### rac-N-cyclopropyl-4-[(1R,2S)-1′-(2-methoxyethyl)-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide

Aqueous NaOH (0.1 mL, 0.100 mmol, 1M in water) was added to a solution of Example 27B (16 mg, 0.027 mmol) in dioxane (0.3 mL) and the reaction was heated to 80° C. After 90 minutes the reaction was diluted with ethyl acetate and acidified with 1M HCl. The layers were separated and the organic phase was washed with brine, dried with MgSO₄, filtered, and concentrated. The crude residue was then taken up in N,N-dimethylformamide (0.300 mL) and HATU (12.38 mg, 0.033 mmol) and N,N-diisopropylethylamine (9.48 μL, 0.054 mmol) were added. After 20 minutes cyclopropylamine (3.10 mg, 0.054 mmol) was added. After stirring for 18 hours the reaction was diluted with ethyl acetate and washed with a saturated aqueous NaHCO₃ solution and brine. The organic phase was dried with MgSO₄, filtered, and concentrated under reduced pressure. The crude residue was purified via flash chromatography (ISCO Combiflash, 2-6% methanol/dichloromethane, 4 g Redisep silica column) to yield the title compound (3.3 mg, 7.39 μmol, 27.2% yield) as solid. ¹H NMR (400 MHz, DMSO-d₆) δ 12.05 (s, 1H), 8.20 (s, 1H), 7.39 (s, 1H), 7.08-6.97 (m, 2H), 6.64 (ddd, J=8.3, 5.9, 2.9 Hz, 1H), 6.46-6.34 (m, 2H), 3.95 (q, J=6.1 Hz, 2H), 3.58 (t, J=5.6 Hz, 2H), 3.53 (s, 3H), 3.24 (s, 3H), 2.87 (t, J=8.4 Hz, 1H), 2.70 (d, J=4.0 Hz, OH), 2.25 (dd, J=8.0, 4.7 Hz, 1H), 1.98 (dd, J=9.0, 4.7 Hz, 1H), 0.62 (td, J=7.0, 4.7 Hz, 2H), 0.48-0.33 (m, 2H); MS (ESI−) m/z 447.2 (M−H)⁻.

### Example 28

### rac-4-{(1R,2S)-1′-[(4-aminophenyl)methyl]-2′oxo-1′,2′-dihydrospiro[cyclopropane-1,3′-indol]-2-yl}-N-cyclopropyl-6-methyl-7-oxo-6,7-dihydro-1H-pyrrolo [2,3-c]pyridine-2-carboxamide

### Example 28A

### rac-ethyl 6-methyl-1-(4-methylbenzene-1-sulfonyl)-4-{(1R,2S)-1′-[(4-nitrophenyl)methyl]-2′-oxo-1′,2′-dihydrospiro[cyclopropane-1,3′-indol]-2-yl}-7-oxo-6,7-dihydro-1H-pyrrolo[2,3-c]pyridine-2-carboxylate

A mixture of Example 27A (0.39 g, 0.734 mmol), 1-(bromomethyl)-4-nitrobenzene (0.182 g, 0.844 mmol, Aldrich), and cesium carbonate (0.311 g, 0.954 mmol) in N,N -dimethylformamide (2.93 mL) was stirred at room temperature. After 3 days the reaction mixture was diluted with ethyl acetate and water. The layers were separated and the organic phase was washed with brine, dried with MgSO₄, filtered, and concentrated under reduced pressure. The crude residue was purified via flash chromatography (ISCO Combiflash, 0-100% ethyl acetate/heptanes, 40 g Redisep silica column) to yield the title compound (352 mg, 0.528 mmol, 72.0% yield) as solid. ¹H NMR (400 MHz, CDCl₃) δ 8.53-8.42 (m, 2H), 8.34-8.18 (m, 2H), 7.57-7.47 (m, 2H), 7.40 (d, J=8.3 Hz, 2H), 7.12 (td, J=7.8, 1.2 Hz, 1H), 7.06 (d, J=1.4 Hz, 1H), 6.78 (td, J=7.6, 1.0 Hz, 1H), 6.73 (d, J=7.8 Hz, 1H), 6.51 (s, 1H), 6.28 (dd, J=7.6, 1.3 Hz, 1H), 5.28-5.05 (m, 2H), 4.44 (q, J=7.1 Hz, 2H), 3.62 (s, 3H), 3.14 (ddd, J=9.2, 7.8, 1.4 Hz, 1H), 2.46 (s, 3H), 2.34 (dd, J=9.1, 4.6 Hz, 1H), 1.94 (dd, J=7.8, 4.7 Hz, 1H), 1.40 (t, J=7.1 Hz, 3H); MS (ESI+) m/z 667.1 (M+H)⁺.

### Example 28B

### rac-6-methyl-1-(4-methylbenzene-1-sulfonyl)-4-{(1R,2S)-1′-[(4-nitrophenyl)methyl]-2′-oxo-1′,2′-dihydrospiro[cyclopropane-1,3′-indol]-2-yl}-7-oxo-6,7-dihydro-1H-pyrrolo[2,3-c]pyridine-2-carboxylic acid

Aqueous NaOH (2.112 mL, 2.112 mmol, 1M in water) was added to a solution of Example 28A (0.352 g, 0.528 mmol) in dioxane (6 mL) and the reaction was heated to 80° C. After 2 hours the reaction mixture was cooled to room temperature and poured into 1M HCl (100 mL). The resulting mixture was filtered and the collected solid was washed twice with water and then dried in a vacuum oven to give the title compound (176 mg, 0.363 mmol, 68.8% yield) as solid. ¹H NMR(501 MHz, DMSO-d₆) δ 13.04 (s, 1H), 12.54 (d, J=2.2 Hz, 1H), 8.27-8.22 (m, 2H), 7.57-7.53 (m, 2H), 7.48 (d, J=1.3 Hz, 1H), 7.01-6.97 (m, 1H), 6.87 (d, J=7.9 Hz, 1H), 6.67 (td, J=7.5, 0.9 Hz, 1H), 6.45 (dd, J=7.5, 1.1 Hz, 1H), 6.36 (d, J=2.1 Hz, 1H), 5.29 (d, J=16.7 Hz, 1H), 5.10 (d, J=16.7 Hz, 1H), 3.56 (s, 3H), 3.05-2.99 (m, 1H), 2.36 (dd, J=7.9, 4.7 Hz, 1H), 2.12 (dd, J=9.0, 4.7 Hz, 1H); MS (ESI−) m/z 482.9 (M−H)⁻.

### Example 28C

### rac-N-cyclopropyl-6-methyl-4-{(1R,2S)-1′-[(4-nitrophenyl)methyl]-2′-oxo-1′,2′-dihydrospiro[cyclopropane-1,3′-indol]-2-yl}-7-oxo-6,7-dihydro-1H-pyrrolo[2,3-c]pyridine-2-carboxamide

N,N-Diisopropylethylamine (0.116 mL, 0.665 mmol) was added to a solution of Example 28B (161 mg, 0.332 mmol) and HATU (145 mg, 0.382 mmol) in N,N-dimethylformamide (2 mL) at room temperature. After 20 minutes cyclopropylamine (37.9 mg, 0.665 mmol) was added. After stirring at room temperature for 2 hours the reaction was diluted with ethyl acetate and washed with a saturated aqueous NaHCO₃ solution and brine. The organic phase was then dried with MgSO₄, filtered, and concentrated under reduced pressure. The crude residue was purified via flash chromatography (ISCO Combiflash, 0-10% methanol/dichloromethane, 40 g Redisep silica column) to yield the title compound (70 mg, 0.134 mmol, 40.2% yield). ¹H NMR(400 MHz, DMSO-d₆) δ 12.11 (s, 1H), 8.31 (d, J=4.2 Hz, 1H), 8.25 (d, J=8.7 Hz, 2H), 7.57 (d, J=8.5 Hz, 2H), 7.48 (d, J=1.2 Hz, 1H), 7.01-6.92 (m, 1H), 6.82 (d, J=7.8 Hz, 1H), 6.71-6.61 (m, 1H), 6.53 (s, 1H), 6.50-6.43 (m, 1H), 5.30 (d, J=16.6 Hz, 1H), 5.07 (dd, J=17.9, 7.6 Hz, 1H), 3.03 (t, J=8.4 Hz, 1H), 2.78 (dq, J=7.4, 4.0 Hz, 1H), 2.39 (dd, J=8.0, 4.8 Hz, 1H), 2.12 (dd, J=9.0, 4.8 Hz, 1H), 0.69 (t, J=4.1 Hz, 2H), 0.57-0.43 (m, 2H); MS (ESI+) m/z 524.2 (M+H)⁺.

### Example 28D

### rac-4-{(1R,2S)-1′-[(4-aminophenyl)methyl]-2′oxo-1′,2′-dihydrospiro[cyclopropane-1,3′-indol]-2-yl}-N-cyclopropyl-6-methyl-7-oxo-6,7-dihydro-1H-pyrrolo [2,3-c]pyridine-2-carboxamide

Ammonium chloride (71.5 mg, 1.337 mmol) and zinc dust (87 mg, 1.337 mmol) were added to a solution of Example 28C (70 mg, 0.134 mmol) in acetone (1.2 mL) and water (0.200 mL) at room temperature. After 1 hour the reaction mixture was filtered through diatomaceous earth, washing with ethyl acetate, and concentrated. The crude residue was then purified via reverse-phase HPLC (5-95% acetonitrile/water with 0.5% trifluoroacetic acid) to yield the title compound (16.8 mg, 0.034 mmol, 25.5% yield) as solid. ¹H NMR (501 MHz, DMSO-d₆) δ 12.07 (s, 1H), 8.29 (d, J=4.2 Hz, 1H), 7.44 (d, J=1.2 Hz, 1H), 7.04-6.93 (m, 3H), 6.83 (d, J=7.9 Hz, 1H), 6.63 (td, J=7.6, 1.1 Hz, 1H), 6.55-6.46 (m, 3H), 6.43 (dd, J=7.6, 1.2 Hz, 1H), 4.94 (s, 2H), 4.90-4.72 (m, 2H), 3.56 (s, 3H), 3.02-2.93 (m, 1H), 2.77 (tq, J=7.7, 4.0 Hz, 1H), 2.30 (dd, J=8.0, 4.7 Hz, 1H), 2.03 (dd, J=8.9, 4.6 Hz, 1H), 0.77-0.60 (m, 2H), 0.59-0.39 (m, 2H); MS (ESI−) m/z 492.0 (M−H)⁻.

### Example 29

### rac-4-[(1R,2S)-7′-bromo-1′-methyl-2′-oxo-1′,2′-dihydrospiro[cyclopropane-1,3′-indol]-2-yl]-N-cyclopropyl-6-methyl-7-oxo-6,7-dihydro-1H-pyrrolo[2,3-c]pyridine-2-carboxamide

### Example 29A

### rac-N-(7-bromo-2-oxoindolin-3-ylidene)-4-methylbenzenesulfonohydrazide

Methanol (100 mL) was added to a mixture of 7-bromoindoline-2,3-dione (9 g, 39.8 mmol) and 4-methylbenzenesulfonohydrazide (7.42 g, 39.8 mmol). The solution was stirred at 60° C. for 16 hours. The solution was cooled to room temperature. The solids were collected by filtration to provide the crude title compound (13.9 g, 35.2 mmol, 88%). ¹H NMR (501 MHz, DMSO-d₆) δ 11.47 (s, 1H), 7.87-7.80 (m, 2H), 7.53 (dd, J=1.0, 8.2 Hz, 1H), 7.45-7.39 (m, 3H), 7.01-6.94 (m, 1H), 2.36 (s, 3H).

### Example 29B

### 7-bromo-3-diazoindolin-2-one

Sodium hydroxide (1 M aqueous, 88 mL, 88 mmol) was added to a solution of Example 29A (13.89 g, 35.2 mmol) in tetrahydrofuran (150 mL). The solution was stirred at 50° C. for 2 hours. The solution was neutralized with acetic acid. The solution was partitioned between ethyl acetate and water and the organic fraction was collected and concentrated. The residue was washed with water and dried in a vacuum to provide the crude title compound (10.1 g, 42.4 mmol, >100% collected) which was carried forward without further purification. ¹H NMR (400 MHz, DMSO-d₆) δ 10.93 (s, 1H), 7.43-7.38 (m, 1H), 7.24 (dd, J=1.0, 8.2 Hz, 1H), 6.93 (d, J=7.8 Hz, 1H).

### Example 29C

### 7-bromo-3-diazo-1-methylindolin-2-one

Iodomethane (447 mg, 3.15 mmol) was added to a solution of Example 29B (500 mg, 2.10 mmol) and potassium carbonate (871 mg, 6.30 mmol) in N,N-dimethyl formamide (5 mL). The solution was stirred at room temperature for 15 hours. Water (5 mL) was added, and the precipitate was collected via filtration to provide the title compound (139 mg, 0.55 mmol, 26%). ¹H NMR (400 MHz, DMSO-d₆) δ 7.46 (dd, J=1.1, 7.6 Hz, 1H), 7.29 (dd, J=1.1, 8.1 Hz, 1H), 7.00-6.89 (m, 1H), 3.54 (s, 3H).

### Example 29D

### rac-4-[(1R,2S)-7′-bromo-1′-methyl-2′-oxo-1′,2′-dihydrospiro[cyclopropane-1,3′-indol]-2-yl]-N-cyclopropyl-6-methyl-7-oxo-6,7-dihydro-1H-pyrrolo[2,3-c]pyridine-2-carboxamide

A solution of Example 29C (139 mg, 0.55 mmol) in dichloromethane (1 mL) was added over 5 minutes to a solution of Example 16B (118 mg, 0.46 mmol) and rhodium(II) acetate dimer (1.0 mg. 2.29 umol) in dichloromethane (2.5 mL) under a nitrogen atmosphere. The solution was stirred for 16 hours at 40° C. then cooled to room temperature and concentrated. Purification via flash chromatography (silica gel, 0-10% methanol/dichloromethane) provided the title compound (310 mg, 0.44 mmol, 95%).¹H NMR (400 MHz, DMSO-d₆) δ 12.11 (s, 1H), 8.30 (d, J=4.1 Hz, 1H), 7.43 (d, J=1.3 Hz, 1H), 7.22 (dd, J=1.1, 8.1 Hz, 1H), 6.66-6.55 (m, 1H), 6.45-6.37 (m, 2H), 3.60 (s, 3H), 3.54 (s, 3H), 3.02-2.93 (m, 1H), 2.73 (td, J=3.7, 7.3 Hz, 1H), 2.32 (dd, J=4.8, 8.1 Hz, 1H), 2.05 (dd, J=4.7, 9.0 Hz, 1H), 0.66 (td, J=4.6, 7.0 Hz, 2H), 0.47 (dt, J=4.3, 6.8 Hz, 2H). MS (ESI) m/z 481.0 (M+H)⁺.

### Example 30

### rac-(1R,2S)-1′-methyl-2-(6-methyl-7-oxo-6,7-dihydro-1H-pyrrolo[2,3-c]pyridin-4-yl)spiro[cyclopropane-1,3′-indol]-2′(1H)-one

### Example 30A

### 4-bromo-1-tosyl-1H-pyrrolo[2,3-c]pyridin-7(6H)-one

A solution of 4M HCl in dioxane (50 mL, 200 mmol) was added to a solution of Example 1C (35 g, 92 mmol) in dioxane at room temperature. The reaction mixture was then stirred at 40° C. for 16 hours. After cooling to room temperature the reaction was concentrated and the resulting residue was triturated with diethyl ether. The solid was collected by filtration, rinsed with additional diethyl ether, and dried to provide the title compound (34.0 g, 93 mmol, 101% yield) as solid. ¹H NMR (501 MHz, CD₃OD) δ 8.05 (d, J=3.3 Hz, 1H), 7.93 (dd, J=8.6, 2.6 Hz, 2H), 7.37 (d, J=7.9 Hz, 2H), 7.24 (d, J=2.7 Hz, 1H), 6.63 (d, J=3.3 Hz, 1H), 2.41 (d, J=2.7 Hz, 3H); LCMS (ESI+) m/z 369.1 (M+H)⁺.

### Example 30B

### 4-bromo-6-methyl-1-tosyl-1H-pyrrolo[2,3-c]pyridin-7(6H)-one

A mixture of Example 30A (34.0 g, 93 mmol), potassium carbonate (32.0 g, 231 mmol) and iodomethane (14.41 mL, 231 mmol) in N,N-dimethylformamide (300 mL) was stirred at room temperature for 16 hours. The reaction mixture was diluted with water and the resulting precipitate was collected by vacuum filtration, rinsed with water, and dried in a vacuum oven to provide the title compound (36.4 g, 94 mmol, 101% yield) as solid. ¹H NMR (300 MHz, DMSO-d₆) δ 8.04 (d, J=3.5 Hz, 1H), 7.96-7.90 (m, 2H), 7.78 (s, 1H), 7.44-7.38 (m, 2H), 6.58 (d, J=3.5 Hz, 1H), 3.38 (s, 3H), 2.37 (s, 3H); LCMS (ESI+) m/z 381.1 (M+H)^{+.}

### Example 30C

### 6-methyl-4-(4,4,5,5-tetramethyl-1,3,2-dioxaborolan-2-yl)-1-tosyl-1H-pyrrolo[2,3-c]pyridin-7(6H)-one

A mixture of Example 30B (53.0 g, 139 mmol), bis(pinacolato)diboron (70.6 g, 278 mmol), potassium acetate (30.0 g, 306 mmol), Pd₂(dba)₃ (3.18 g, 3.48 mmol) and X-phos (6.63 g, 13.90 mmol) in dioxane (500 mL) was stirred at 90° C. for 16 hours under an argon atmosphere. The reaction was concentrated and the resulting residue was purified by chromatography (silica gel, 25-80% ethyl acetate in petroleumether). The resulting material was triturated with a minimal amount of hexanes (30 mL) and the particulate solid was collected by filtration, rinsed with a minimal amount of hexanes, and dried to constant mass to afford the title compound (55 g, 128 mmol, 92% yield) as solid. ¹H NMR (300 MHz, DMSO-d₆) δ 7.96 (d, J=3.4 Hz, 1H), 7.93-7.83 (m, 2H), 7.70 (s, 1H), 7.47-7.25 (m, 2H), 6.80 (d, J=3.4 Hz, 1H), 3.42 (s, 3H), 2.36 (s, 3H), 1.28 (s, 12H).LCMS (ESI+) m/z 428.7 (M+H)⁺.

### Example 30D

### 6-methyl-1-tosyl-4-vinyl-1H-pyrrolo[2,3-c]pyridin-7(6H)-one

Example 30C (2.5 g, 5.84 mmol), tris(dibenzylideneacetone)dipalladium(0) (0.053 g, 0.058 mmol), 1,3,5,7-tetramethyl-6-phenyl-2,4,8-trioxa-6-phosphaadamantane (0.041 g, 0.140 mmol, Aldrich), and potassium phosphate (3.10 g, 14.59 mmol) were combined under a nitrogen atmosphere. The reaction flask was then charged with dioxane (23.35 mL), bromoethene (17.51 mL, 17.51 mmol, 1M in tetrahydrofuran, Aldrich), and water (5.84 mL) and heated to 60° C. After 2 hours the reaction was diluted with ethyl acetate and washed with water and brine. The organic phase was dried with MgSO₄, filtered, and concentrated under reduced pressure. The crude residue was then purified via flash chromatography (ISCO Combiflash, 50-100% ethyl acetate/heptanes, 80 g Redisep silic column) to yield the title compound (1.35 g, 4.11 mmol, 70.4% yield) as solid. ¹H NMR (400 MHz, CDCl₃) δ 8.05-7.99 (m, 2H), 7.96 (d, J=3.5 Hz, 1H), 7.35-7.29 (m, 2H), 7.09 (d, J=0.7 Hz, 1H), 6.71 (d, J=3.6 Hz, 1H), 6.60 (ddd, J=17.6, 11.2, 0.8 Hz, 1H), 5.59 (dd, J=17.7, 0.9 Hz, 1H), 5.25 (dd, J=11.2, 0.8 Hz, 1H), 3.53 (s, 3H), 2.41 (s, 3H); MS (ESI+) m/z 329.0 (M+H)⁺.

### Example 30E

### rac-(1R,2S)-1′-methyl-2-[6-methyl-1-(4-methylbenzene-1-sulfonyl)-7-oxo-6,7-dihydro-1H-pyrrolo[2,3-c]pyridin-4-yl]spiro[cyclopropane-1,3′-indol]-2′(1H)-one

A solution of 3-diazo-1-methylindolin-2-one (0.975 g, 5.63 mmol, prepared as described in Org. Lett., 2016, 18, 1358-1361) in dichloromethane (10 mL) was added over 3 hours via syringe pump to a solution of Example 30D (1.54 g, 4.69 mmol) and rhodium(II) acetate dimer (10.36 mg, 0.023 mmol) in dichloromethane (35 mL) at room temperature. After 30 minutes the reaction was concentrated and the crude residue was purified via flash chromatography (ISCO Combiflash, 60-100% ethyl acetate/heptanes, 120 g Redisep silica column) to yield the title compound (2.225 g, 4.70 mmol, 100% yield) as solid. ¹H NMR (400 MHz, CDCl₃) δ 7.98-7.91 (m, 2H), 7.69 (d, J=3.4 Hz, 1H), 7.35-7.25 (m, 2H), 7.18 (td, J=7.7, 1.2 Hz, 1H), 6.90 (d, J=7.8 Hz, 1H), 6.70 (td, J=7.6, 1.0 Hz, 1H), 6.17 (dd, J=7.5, 1.1 Hz, 1H), 6.01 (d, J=3.5 Hz, 1H), 3.53 (s, 3H), 3.37 (s, 3H), 3.03 (ddd, J=9.0, 7.7, 1.5 Hz, 1H), 2.41 (s, 3H), 2.21 (dd, J=8.9, 4.5 Hz, 1H), 1.82 (dd, J=7.7, 4.5 Hz, 1H); MS (ESI+) m/z 474.2 (M+H)⁺.

### Example 30F

### rac-(1R,2S)-1′-methyl-2-(6-methyl-7-oxo-6,7-dihydro-1H-pyrrolo[2,3-c]pyridin-4-yl)spiro[cyclopropane-1,3′-indol]-2′(1H)-one

Aqueous NaOH (0.253 mL, 0.253 mmol, 1M in water) was added to a solution of Example 30E (40 mg, 0.084 mmol) in dioxane (0.75 mL) at room temperature. The reaction was then heated to 80° C. for 1 hour. After cooling to room temperature the reaction mixture was diluted with ethyl acetate and 1M aqueous HCl. The layers were separated and the organic phase was washed with brine, dried with MgSO₄, filtered, and concentrated under reduced pressure. The crude residue was purified via flash chromatography (ISCO Combiflash, 2-5% methanol/dichloromethane, 12 g Redisep Gold silica column) to yield the title compound (18.8 mg, 0.059 mmol, 69.7% yield) as solid. ¹H NMR (400 MHz, DMSO-d₆) δ 11.85 (s, 1H), 7.33 (d, J=1.3 Hz, 1H), 7.05 (td, J=7.7, 1.2 Hz, 1H), 7.02 (t, J=2.7 Hz, 1H), 6.97-6.92 (m, 1H), 6.64 (td, J=7.5, 1.0 Hz, 1H), 6.35 (dd, J=7.4, 1.1 Hz, 1H), 5.75-5.66 (m, 1H), 3.52 (s, 3H), 3.23 (s, 3H), 2.88 (ddd, J=9.0, 7.8, 1.3 Hz, 1H), 2.20 (dd, J=7.9, 4.6 Hz, 1H), 1.96 (dd, J=9.0, 4.6 Hz, 1H); MS (ESI−) m/z 318.0 (M−H)⁻.

### Example 31

### rac-(1R,2S)-2-(2-cyclopropyl-6-methyl-7-oxo-6,7-dihydro-1H-pyrrolo[2,3-c]pyridin-4-yl)-1′-methylspiro[cyclopropane-1,3′-indol]-2′(1H)-one

### Example 31A

### 2-iodo-6-methyl-1-tosyl-4-vinyl-1H-pyrrolo[2,3-c]pyridin-7(6H)-one

A solution of lithium N,N-diisopropylamide (LDA; 4.60 mL, 9.20 mmol, 2M in tetrahydrofuran/heptane, Aldrich) was added dropwise to a solution of Example 30D (2.015 g, 6.14 mmol) in tetrahydrofuran (40 mL) at −78° C. After 45 minutes a solution of iodine (2.336 g, 9.20 mmol) in tetrahydrofuran (10 mL) was added to the reaction mixture dropwise before warming to room temperature. After 1 hour the reaction was quenched with a saturated aqueous Na₂S₂O₃ solution and the resulting mixture was extracted with ethyl acetate. The organic phase was washed with brine, dried with MgSO₄, filtered, and concentrated under reduced pressure. The crude material was purified via flash chromatography (ISCO Combilfash, 0-20% ethyl acetate/dichloromethane, 120 g Redisep silica column) to yield the title compound (1.423 g, 3.13 mmol, 51.0% yield) as solid. ¹H NMR (400 MHz, CDCl₃) δ 8.37-8.14 (m, 2H), 7.44-7.32 (m, 2H), 7.10 (d, J=10.8 Hz, 2H), 6.54 (ddd, J=17.7, 11.2, 0.7 Hz, 1H), 5.54 (dd, J=17.7, 0.7 Hz, 1H), 5.24 (dd, J=11.2, 0.7 Hz, 1H), 3.56 (s, 3H), 2.44 (s, 3H); MS (ESI+) m/z 455.0 (M+H)⁺.

### Example 31B

### rac-(1R,2S)-2-[2-iodo-6-methyl-1-(4-methylbenzene-1-sulfonyl)-7-oxo-6,7-dihydro-1H-pyrrolo[2,3-c]pyridin-4-yl]-1′-methylspiro[cyclopropane-1,3′-indol]-2′(1H)-one

A solution of 3-diazo-1-methylindolin-2-one (0.651 g, 3.76 mmol, prepared as described in Org. Lett., 2016, 18, 1358-1361) in dichloromethane (10 mL) was added over 1 hour via syringe pump to a solution of Example 31A (1.423 g, 3.13 mmol) and rhodium(II) acetate dimer (6.92 mg, 0.016 mmol) in dichloromethane (50 mL) at room temperature. After 90 minutes the reaction was concentrated under reduced pressure and the crude residue was purified via flash chromatography (ISCO Combiflash, 50-100% ethyl acetate/heptanes, 120 g Redsep silica column) to yield the title compound (1.73 g, 2.89 mmol, 92% yield) as solid. ¹H NMR (500 MHz, CDCl₃) δ 8.29-8.21 (m, 2H), 7.38 (d, J=8.1 Hz, 2H), 7.23 (td, J=7.7, 1.2 Hz, 1H), 6.99-6.88 (m, 2H), 6.78 (td, J=7.6, 1.0 Hz, 1H), 6.51 (s, 1H), 6.24 (dd, J=7.6, 1.3 Hz, 1H), 3.56 (s, 3H), 3.40 (s, 3H), 3.00 (ddd, J=9.1, 7.7, 1.4 Hz, 1H), 2.46 (s, 3H), 2.22 (dd, J=9.0, 4.5 Hz, 1H), 1.81 (dd, J=7.7, 4.5 Hz, 1H); MS (ESI+) m/z 600.1 (M+H)⁺.

### Example 31C

### rac-(1R,2S)-2-[2-cyclopropyl-6-methyl-1-(4-methylbenzene-1-sulfonyl)-7-oxo-6,7-dihydro-1H-pyrrolo[2,3-c]pyridin-4-yl]-1′-methylspiro[cyclopropane-1,3′-indol]-2′(1H)-one

Example 31B (40 mg, 0.067 mmol), cyclopropylboronic acid (17.20 mg, 0.200 mmol, Aldrich), tris(dibenzylideneacetone)dipalladium(0) (1.222 mg, 1.335 μmol), 1,3,5,7-tetramethyl-6-phenyl-2,4,8-trioxa-6-phosphaadamantane (0.936 mg, 3.20 μmol, Aldrich), and potassium phosphate (35.4 mg, 0.167 mmol) were combined in a 4 mL vial with stir bar and septum cap. The vial was evacuated and backfilled with nitrogen three times and then charged with dioxane (1068 μL) and water (267 μL). The reaction was heated at 60° C. for 18 hours then diluted with ethyl acetate and washed with water and brine. The organic phase was dried with MgSO₄, filtered, and concentrated under reduced pressure. The crude residue was purified via reverse-phase HPLC (5-95% acetonitrile/water with 0.1% trifluoroacetic acid) to yield the title compound (23.6 mg, 0.046 mmol, 68.9% yield) as solid. ¹H NMR (400 MHz, CDCl₃) δ 8.04-7.94 (m, 2H), 7.31 (d, J=8.1 Hz, 2H), 7.20 (td, J=7.8, 1.2 Hz, 1H), 6.92 (d, J=7.8 Hz, 1H), 6.72 (td, J=7.6, 1.0 Hz, 1H), 6.19-6.11 (m, 1H), 5.59 (d, J=0.9 Hz, 1H), 3.55 (d, J=4.0 Hz, 3H), 3.40 (s, 3H), 3.00 (ddd, J=9.1, 7.7, 1.4 Hz, 1H), 2.57-2.45 (m, 1H), 2.42 (s, 3H), 2.24 (dd, J=9.0, 4.6 Hz, 1H), 1.82 (dd, J=7.7, 4.5 Hz, 1H), 1.07-0.91 (m, 2H), 0.54 (ddd, J=11.5, 5.3, 2.5 Hz, 1H), 0.44 (ddd, J=9.6, 5.3, 2.0 Hz, 1H); MS (ESI+) m/z 514.2 (M+H)⁺.

### Example 31D

### rac-(1R,2S)-2-(2-cyclopropyl-6-methyl-7-oxo-6,7-dihydro-1H-pyrrolo[2,3-c]pyridin-4-yl)-1′-methylspiro[cyclopropane-1,3′-indol]-2′(1H)-one

A mixture of LiOH (3.5 mg, 0.141 mmol) and Example 31C (24 mg, 0.047 mmol) in dioxane (0.45 mL) and water (0.15 mL) was heated at 100° C. for 18 hours. After cooling to room temperature the reaction mixture was partitioned between ethyl acetate and 1M HCl. The layers were separated and the organic phase was washed with brine, dried with MgSO₄, filtered, and concentrated under reduced pressure. The crude residue was then purified via reverse phase HPLC (5-95% acetonitrile/water with 0.1% trifluoroacetic acid) to yield the title compound (5.9 mg, 0.016 mmol, 35.1% yield) as solid. ¹H NMR (501 MHz, DMSO-d₆) δ 11.62 (s, 1H), 7.29 (d, J=1.2 Hz, 1H), 7.09 (td, J=7.7, 1.2 Hz, 1H), 6.98 (d, J=7.7 Hz, 1H), 6.68 (t, J=7.5 Hz, 1H), 6.39 (d, J=7.4 Hz, 1H), 5.39 (d, J=2.1 Hz, 1H), 3.50 (s, 3H), 3.24 (s, 3H), 2.82 (t, J=8.5 Hz, 1H), 2.20 (dd, J=7.9, 4.6 Hz, 1H), 1.94 (dd, J=9.0, 4.6 Hz, 1H), 1.81 (tt, J=8.4, 5.1 Hz, 1H), 0.87-0.73 (m, 2H), 0.66-0.48 (m, 2H); MS (ESI+) m/z 360.1 (M+H)⁺.

### Example 32

### rac-tert-butyl 3-{6-methyl-4-[(1R,2S)-1′-methyl-2′oxo-1′,2′-dihydrospiro[cyclopropane-1,3′-indol]-2-yl]-7-oxo-6,7-dihydro-1H-pyrrolo [2,3-c]pyridin-2-yl}azetidine-1-carboxylate

### Example 32A

### rac-tert-butyl 3-{6-methyl-1-(4-methylbenzene-1-sulfonyl)-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridin-2-yl}azetidine-1-carboxylate

Zinc dust (21.81 mg, 0.334 mmol, oven-dried, Aldrich) was added under a nitrogen atmosphere to a 4 mL vial with stir bar and septum cap. The vial was charged with tetrahydrofuran (0.25 mL) and 1,2-dibromoethane (2.156 μL, 0.025 mmol) and then placed in a preheated metal heating block at 65° C. for 10 minutes. After cooling to room temperature, chlorotrimethylsilane (3.20 μL, 0.025 mmol) was added, followed after 30 minutes by the addition of tent-butyl 3-iodoazetidine-1-carboxylate (46.6 μL, 0.250 mmol). The reaction mixture was then stirred at room temperature for 45 minutes. Tris(dibenzylideneacetone)dipalladium(0) (3.82 mg, 4.17 μmol), 1,3,5,7-tetramethyl-6-phenyl-2,4,8-trioxa-6-phosphaadamantane (2.93 mg, 10.01 μmol, Aldrich), and Example 31B (100 mg, 0.167 mmol) were combined in a separate 4 mL vial with stir bar and septum cap. The vial was evacuated and backfilled three times with nitrogen and then charged with tetrahydrofuran (0.75 mL). After stirring at room temperature for 5 minutes the prepared zinc reagent solution was added via syringe. The reaction was then heated to 65° C. After stirring for 18 hours the reaction was diluted with ethyl acetate and washed with a saturated aqueous NaHCO₃ solution and brine. The organic phase was then dried with MgSO₄, filtered, and concentrated under reduced pressure. The crude residue was purified via flash chromatography (ISCO Combiflash, 60-100% ethyl acetate/heptanes, 12 g Redisep Gold silica column) to yield the title compound (11.6 mg, 0.018 mmol, 11.06% yield) as solid. ¹H NMR (500 MHz, CDCl₃) δ 7.91-7.85 (m, 2H), 7.33 (d, J=8.2 Hz, 2H), 7.21 (td, J=7.7, 1.2 Hz, 1H), 7.03 (d, J=1.5 Hz, 1H), 6.95 (d, J=7.8 Hz, 1H), 6.77-6.68 (m, 1H), 6.20-6.15 (m, 1H), 5.91 (s, 1H), 4.27 (d, J=9.0 Hz, 3H), 3.92-3.81 (m, 1H), 3.68 (s, 1H), 3.54 (s, 3H), 3.44 (s, 3H), 3.03 (ddd, J=9.2, 7.7, 1.5 Hz, 1H), 2.45 (s, 3H), 2.30 (dd, J=8.9, 4.6 Hz, 1H), 1.87 (dd, J=7.6, 4.6 Hz, 1H), 1.53 (s, 9H); LC/MS (ESI+) m/z 629.2 (M+H)⁺.

### Example 32B

### rac-tert-butyl 3-{6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo [2,3-c]pyridin-2-yl}azetidine-1-carboxylate

Aqueous NaOH (0.070 mL, 0.070 mmol, 1M in water) was added to a solution of Example 32A (11 mg, 0.017 mmol) in dioxane (0.25 mL) and the mixture was then heated at 100° C. for 18 hours. After cooling to room temperature the reaction was diluted with ethyl acetate, washed with a saturated aqueous NH₄Cl solution, dried with MgSO₄, filtered, and concentrated under reduced pressure. The crude residue was then purified via reverse phase HPLC (5-95% acetonitrile/water with 0.1% trifluoroacetic acid) to yield the title compound (3.7 mg, 7.80 μmol, 44.6% yield) as solid. ¹H NMR (500 MHz, DMSO-d₆) δ 11.99 (d, J=2.2 Hz, 1H), 7.37 (d, J=1.3 Hz, 1H), 7.22 (s, 1H), 7.14-7.08 (m, 2H), 7.03-6.96 (m, 2H), 6.71 (td, J=7.5, 1.0 Hz, 1H), 6.44 (dd, J=7.4, 1.1 Hz, 1H), 5.77 (d, J=2.1 Hz, 1H), 4.03 (s, 3H), 3.87-3.70 (m, 3H), 2.90 (ddd, J=9.1, 7.9, 1.3 Hz, 1H), 2.26 (dd, J=8.0, 4.6 Hz, 1H), 1.99 (dd, J=9.0, 4.6 Hz, 1H), 1.39 (s, 9H); MS (ESI+) m/z 474.9 (M+H)⁺.

### Example 33

### rac-(1R,2S)-1′-methyl-2-[6-methyl-2-(1-methyl-1H-pyrazol-4-yl)-7-oxo-6,7-dihydro-1H-pyrrolo[2,3-c]pyridin-4-yl]spiro[cyclopropane-1,3′-indol]-2′(1H)-one

### Example 33A

### 4-bromo-2-iodo-7-methoxy-1-tosyl-1H-pyrrolo[2,3-c]pyridine

A lithium N,N-diisopropylamide solution (18.36 mL, 36.7 mmol, 2M in tetrahydrofuran/heptanes) was added dropwise to a mixture of Example 1C (10 g, 26.2 mmol) in tetrahydrofuran (100 mL) at −78° C. After stirring for 90 minutes a solution of iodine (15.31 g, 60.3 mmol) in tetrahydrofuran (40 mL) was added dropwise. After 2 hours the reaction mixture was quenched with an aqueous Na₂S₂O₃ solution, extracted four times with dichloromethane, dried over Na₂SO₄, and concentrated in vacuo. The residue was triturated with dichloromethane and the filtered solid was collected and dried in vacuo to give the title compound (8.5 g, 15.08 mmol, 57.5% yield) as solid. ¹H NMR (500 MHz, DMSO-d₆) δ 7.98 (s, 1H), 7.91 (d, J=8.2 Hz, 2H), 7.52 (d, J=8.1 Hz, 2H), 7.16 (s, 1H), 3.82 (s, 3H), 2.41 (s, 3H); MS (ESI+) m/z 506.8 (M+H)⁺.

### Example 33B

### 4-methylphenyl 4-bromo-2-iodo-7-oxo-6,7-dihydro-1H-pyrrolo[2,3-c]pyridine-1-sulfonate

Sodium iodide (4.73 g, 31.5 mmol) and chlorotrimethylsilane (3.43 g, 31.5 mmol) were sequentially added to a mixture of Example 33A (10 g, 19.72 mmol) in acetonitrile (250 mL). The reaction was stirred at room temperature for 30 minutes and then water (0.178 mL, 9.86 mmol) was added. The resulting mixture was then stirred at 65° C. for 3 hours. After cooling to room temperature, the mixture was filtered and the resulting solid was washed with dichloromethane and dried in vacuo to provide the title compound. The dichloromethane extract was concentrated and then purified by column chromatography (silica gel, dichloromethane:methanol=20:1) to provide a second batch of the product. The combined batches provided the product (7.7 g, 15.12 mmol, 77% yield). ¹H NMR (500 MHz, DMSO-d₆) δ 11.51 (s, 1H), 8.10-8.05 (m, 2H), 7.51-7.44 (m, 2H), 7.37 (s, 1H), 6.94 (s, 1H), 2.40 (s, 3H); MS (ESI−) m/z 490.8 (M−H)⁻.

### Example 33C

### 4-bromo-2-iodo-6-methyl-1-tosyl-1H-pyrrolo[2,3-c]pyridin-7(6H)-one

Sodium hydride (60% dispersion in mineral oil, 0.812 g, 20.30 mmol) was added in portions to a solution of Example 33B (7.7 g, 15.62 mmol) in N,N-dimethylformamide (100 mL) at 0° C. After 30 minutes iodomethane (1.269 mL, 20.30 mmol) was added dropwise to the mixture. The reaction was stirred at room temperature for 3 hours and then quenched with an aqueous NH₄Cl solution. The resulting suspension was filtered and the filter cake was dissolved in dichloromethane, dried over Na₂SO₄, filtered and concentrated under reduced pressure. The residue was dissolved in dichloromethane (˜150 mL), and then methanol (˜150 mL) was added. The solution was partially concentrated until a significant amount of solid precipitated. The formed solid was collected by filtration and dried under vacuum to give the title compound (6.2 g, 11.98 mmol, 77% yield) as solid. ¹H NMR (500 MHz, DMSO-d₆) δ 8.12-8.04 (m, 2H), 7.82 (s, 1H), 7.52-7.40 (m, 2H), 6.95 (s, 1H), 3.40 (s, 3H), 2.41 (s, 3H); MS (ESI+) m/z 506.8 (M+H)⁺.

### Example 33D

### 4-bromo-6-methyl-2-(1-methyl-1H-pyrazol-4-yl)-1-tosyl-1H-pyrrolo[2,3-c]pyridin-7(6H)-one

A mixture of Example 33C (5 g, 9.86 mmol), 1-methyl-4-(4,4,5,5-tetramethyl-1,3,2-dioxaborolan-2-yl)-1H-pyrazole (2.154 g, 10.35 mmol, SCRC), 1,3,5,7-tetramethyl-8-phenyl-2,4,6-trioxa-8-phosphaadamantane (0.288 g, 0.986 mmol, Aldrich), tris(dibenzylideneacetone)dipalladium(0) (0.226 g, 0.246 mmol), and potassium phosphate dibasic (2.58 g, 14.79 mmol) in dioxane (60 mL) and water (15 mL) was heated to 50° C. for 3 hours. The reaction mixture was diluted with water and extracted with ethyl acetate. The organic layer was washed with brine, dried over Na₂SO₄, filtered, and concentrated. The residue was purified by flash column chromatography (silica gel, ethyl acetate/petroleum ether=1:1 to 2:1) to give the title compound (4 g, 8.67 mmol, 88% yield) as solid. ¹H NMR (400 MHz, DMSO-d₆) δ 7.97 (d, J=0.8 Hz, 1H), 7.88-7.73 (m, 3H), 7.58 (d, J=0.8 Hz, 1H), 7.43-7.31 (m, 2H), 6.47 (s, 1H), 3.85 (s, 3H), 3.37 (s, 3H), 2.36 (s, 3H); MS (ESI+) m/z 461.0 (M+H)⁺.

### Example 33E

### 6-methyl-2-(1-methyl-1H-pyrazol-4-yl)-4-(4,4,5,5-tetramethyl-1,3,2-dioxaborolan-2-yl)-1-tosyl-1H-pyrrolo[2,3-c]pyridin-7(6H)-one

A mixture of Example 33D (3.48 g, 7.54 mmol), 4,4,4′,4′,5,5,5′,5′-octamethyl-2,2′-bis(1,3,2-dioxaborolane) (5.75 g, 22.63 mmol), potassium acetate (2.59 g, 26.4 mmol), tris(dibenzylideneacetone)dipalladium(0) (0.138 g, 0.151 mmol) and 2-(dicyclohexylphosphino)-2′,4′,6′-triisopropylbiphenyl (0.302 g, 0.634 mmol) in dioxane (50 mL) was degassed and backfilled with nitrogen before heating at 70° C. for 21 hours. The mixture was then partitioned between ethyl acetate and water and the aqueous layer was extracted with ethyl acetate. The combined organic phases were concentrated and the resulting residue was purified by flash chromatography (20 g silica gel column, 20% to 100% ethyl acetate/heptanes) to give the title compound (3.8 g, 7.47 mmol, 99% yield) as solid. ¹H NMR (400 MHz, DMSO-d₆) δ 7.96 (d, J=0.7 Hz, 1H), 7.77 (dd, J=6.7, 1.7 Hz, 3H), 7.53 (d, J=0.8 Hz, 1H), 7.40-7.34 (m, 2H), 6.65 (s, 1H), 3.87 (s, 3H), 3.43 (s, 3H), 2.37 (s, 3H), 1.26 (s, 12H); MS (ESI+) m/z 474.9 (M+H)⁺.

### Example 33F

### 6-methyl-2-(1-methyl-1H-pyrazol-4-yl)-1-tosyl-4-vinyl-1H-pyrrolo[2,3-c]pyridin-7(6H)-one

Example 33E (500 mg, 0.983 mmol), tris(dibenzylideneacetone)dipalladium(0) (18.01 mg, 0.020 mmol), 1,3,5,7-tetramethyl-6-phenyl-2,4,8-trioxa-6-phosphaadamantane (13.80 mg, 0.047 mmol) and potassium phosphate tribasic (522 mg, 2.459 mmol) were combined in a 20 mL vial with stir bar and septum cap. The vial was evacuated and backfilled three times with nitrogen before being charged with dioxane (3934 μL), bromoethene (2950 μL, 2.95 mmol, 1M in tetrahydrofuran, Aldrich), and water (983 μL). The resulting mixture was then heated at 60° C. for 4 hours. After cooling to room temperature the reaction was diluted with ethyl acetate and washed with water and brine. The organic phase was then dried with MgSO₄, filtered, and concentrated under reduced pressure. The crude residue was then purified via flash chromatography (ISCO Combiflash, 50-100% ethyl acetate/heptanes, 40 g Redisep silica column) to yield the title compound (352 mg, 0.862 mmol, 88% yield) as solid. ¹H NMR (400 MHz, CDCl₃) δ 7.98-7.88 (m, 2H), 7.63 (s, 1H), 7.56 (s, 1H), 7.30 (d, J=8.3 Hz, 2H), 7.18 (s, 1H), 6.68-6.51 (m, 2H), 5.58 (dd, J=17.7, 0.9 Hz, 1H), 5.30-5.17 (m, 1H), 3.99 (s, 3H), 3.60 (s, 3H), 2.43 (s, 3H); MS (ESI+) m/z 409.0 (M+H)⁺.

### Example 33G

### rac-(1R,2S)-1′-methyl-2-[6-methyl-1-(4-methylbenzene-1-sulfonyl)-2-(1-methyl-1H-pyrazol-4-yl)-7-oxo-6,7-dihydro-1H-pyrrolo[2,3-c]pyridin-4-yl]spiro[cyclopropane-1,3′-indol]-2′(1H)-one

A solution of 3-diazo-1-methylindolin-2-one (338 mg, 1.951 mmol, prepared as described in Org. Lett., 2016, 18, 1358-1361) in 1,2-dichloroethane (5 mL) was added over 12 hours via syringe pump to a solution of Example 33F (613 mg, 1.501 mmol) and rhodium(II) acetate dimer (3.32 mg, 7.50 μmol) in 1,2-dichloroethane (25 mL) at 90° C. After heating for 4 hours the reaction was concentrated and the crude residue was purified via flash chromatography (ISCO Combiflash, 0-5% methanol/dichloromethane, 80 g Redisep Gold silica column) to yield the title compound (0.277 g, 0.500 mmol, 33.3% yield) as solid. ¹H NMR (501 MHz, DMSO-d₆) δ 7.79 (s, 1H), 7.74-7.69 (m, 2H), 7.65 (d, J=1.2 Hz, 1H), 7.37 (d, J=0.8 Hz, 1H), 7.37-7.31 (m, 2H), 7.13 (td, J=7.7, 1.2 Hz, 1H), 7.03-6.95 (m, 1H), 6.72 (td, J=7.6, 1.0 Hz, 1H), 6.36 (dd, J=7.6, 1.1 Hz, 1H), 5.96 (s, 1H), 5.74 (s, 1H), 3.81 (s, 3H), 3.45 (s, 3H), 3.22 (s, 3H), 2.85 (ddd, J=9.0, 7.7, 1.2 Hz, 1H), 2.36 (s, 3H), 2.21 (dd, J=7.8, 4.7 Hz, 1H), 1.96 (dd, J=9.0, 4.7 Hz, 1H); MS (ESI+) m/z 554.1 (M+H)⁺.

### Example 33H

### rac-(1R,2S)-1′-methyl-2-[6-methyl-2-(1-methyl-1H-pyrazol-4-yl)-7-oxo-6,7-dihydro-1H-pyrrolo[2,3-c]pyridin-4-yl]spiro[cyclopropane-1,3′-indol]-2′(1H)-one

Aqueous NaOH (1.00 mL, 1.00 mmol, 1M in water) was added to a solution of Example 33G (0.277 g, 0.500 mmol) in dioxane (3 mL) and the reaction was heated at 80° C. for 10 hours. After cooling to room temperature the reaction was diluted with dichloromethane and washed with a saturated aqueous NH₄Cl solution. The combined organic phase was dried with MgSO₄, filtered, and concentrated under reduced pressure. The crude residue was purified via flash chromatography (ISCO Combiflash, 2-10% methanol/dichloromethane, 40 g Redisep Gold silica column) to yield the title compound (89 mg, 0.223 mmol, 44.5% yield) as solid. ¹H NMR (400 MHz, DMSO-d₆) δ 12.00 (s, 1H), 8.10 (s, 1H), 7.86 (d, J=0.7 Hz, 1H), 7.35 (d, J=1.2 Hz, 1H), 7.07 (td, J=7.7, 1.2 Hz, 1H), 6.97 (d, J=7.7 Hz, 1H), 6.69 (td, J=7.5, 1.1 Hz, 1H), 6.44 (dd, J=7.5, 1.2 Hz, 1H), 5.95 (d, J=2.0 Hz, 1H), 3.78 (s, 3H), 3.55 (s, 3H), 3.26 (s, 3H), 2.95-2.84 (m, 1H), 2.25 (dd, J=7.9, 4.6 Hz, 1H), 1.97 (dd, J=8.9, 4.6 Hz, 1H); MS (ESI+) m/z 400.1 (M+H)⁺.

### Example 34

### (1S,2R)-1′-methyl-2-[6-methyl-2-(1-methyl-1H-pyrazol-4-yl)-7-oxo-6,7-dihydro-1H-pyrrolo[2,3-c]pyridin-4-yl]spiro[cyclopropane-1,3′-indol]-2′(1H)-one

The enantiomers of Example 33H (89 mg) were separated by preparative chiral SFC using a Pic SFC Hybrid 10-100 instrument. The mobile phase consisted of 30% 80:20 isopropanol:acetonitrile in CO₂ (83 mL/minute, 150 bar, 30° C.) and the stationary phase was a. YMC-SA, 30×150 mm, column (5 p.m particles). The title compound (30 mg, 0.075 mmol, 33.7% yield) was isolated as solid with retention time of 5.2-6.8 minutes. ¹H NMR (400 MHz, DMSO-d₆) δ 12.00 (s, 1H), 8.10 (s, 1H), 7.86 (d, J=0.7 Hz, 1H), 7.35 (d, J=1.2 Hz, 1H), 7.07 (td, J=7.7, 1.2 Hz, 1H), 6.97 (d, J=7.7 Hz, 1H), 6.69 (td, J=7.5, 1.1 Hz, 1H), 6.44 (dd, J=7.5, 1.2 Hz, 1H), 5.95 (d, J=2.0 Hz, 1H), 3.78 (s, 3H), 3.55 (s, 3H), 3.26 (s, 3H), 2.95-2.84 (m, 1H), 2.25 (dd, J=7.9, 4.6 Hz, 1H), 1.97 (dd, J=8.9, 4.6 Hz, 1H); MS (ESI+) m/z 400.1 (M+H)⁺.

### Example 35

### (1R,2S)-1′-methyl-2-[6-methyl-2-(1-methyl-1H-pyrazol-4-yl)-7-oxo-6,7-dihydro-1H-pyrrolo[2,3-c]pyridin-4-yl]spiro[cyclopropane-1,3′-indol]-2′(1H)-one

Following the procedure of Example 34, the title compound (30 mg, 0.075 mmol, 33.7% yield) was isolated as solid with retention time of 7.2-9.1 minutes. ¹H NMR (400 MHz, DMSO-d₆) δ 12.00 (s, 1H), 8.10 (s, 1H), 7.86 (d, J=0.7 Hz, 1H), 7.35 (d, J=1.2 Hz, 1H), 7.07 (td, J=7.7, 1.2 Hz, 1H), 6.97 (d, J=7.7 Hz, 1H), 6.69 (td, J=7.5, 1.1 Hz, 1H), 6.44 (dd, J=7.5, 1.2 Hz, 1H), 5.95 (d, J=2.0 Hz, 1H), 3.78 (s, 3H), 3.55 (s, 3H), 3.26 (s, 3H), 2.95-2.84 (m, 1H), 2.25 (dd, J=7.9, 4.6 Hz, 1H), 1.97 (dd, J=8.9, 4.6 Hz, 1H); MS (ESI+) m/z 400.1 (M+H)⁺.

### Example 36

### rac-(1R,2S)-2-[2-(1,3-dimethyl-1H-pyrazol-4-yl)-6-methyl-7-oxo-6,7-dihydro-1H-pyrrolo[2,3-c]pyridin-4-yl]-1′-methylspiro[cyclopropane-1,3′-indol]-2′(1H)-one

### Example 36A

### rac-(1R,2S)-2-[2-(1,3-dimethyl-1H-pyrazol-4-yl)-6-methyl-1-(4-methylbenzene-1-sulfonyl)-7-oxo-6,7-dihydro-1H-pyrrolo[2,3-c]pyridin-4-yl]-1′-methylspiro[cyclopropane-1,3′-indol]-2′(1H)-one

1,3-Dimethyl-4-(4,4,5,5-tetramethyl-1,3,2-dioxaborolan-2-yl)-1H-pyrazole (33.3 mg, 0.150 mmol), Example 31B (75 mg, 0.125 mmol), tris(dibenzylideneacetone)dipalladium(0) (2.291 mg, 2.502 μmol), 1,3,5,7-tetramethyl-6-phenyl-2,4,8-trioxa-6-phophaadamantane (1.755 mg, 6.01 μmol, Aldrich), and potassium phosphate tribasic (66.4 mg, 0.313 mmol) were combined in a 4 mL vial with stir bar and septum cap. The vessel was evacuated and backfilled three times with nitrogen before being charged with dioxane (1.0 mL) and water (0.2 mL). The vial was then heated at 60° C. in a metal heating block. After 18 hours the reaction was diluted with ethyl acetate and washed with water and brine. The organic phase was dried with MgSO₄, filtered, and concentrated under reduced pressure. The crude residue was purified via flash chromatography (ISCO Combiflash, 0-10% methanol/dichloromethane, 12 g Redisep Gold silica column to yield the title compound (9.6 mg, 0.017 mmol, 13.52% yield) as solid. ¹H NMR (400 MHz, CDCl₃) δ 7.73-7.61 (m, 2H), 7.39 (s, 1H), 7.32 (dd, J=7.8, 1.3 Hz, 1H), 7.25 (t, J=7.5 Hz, 3H), 6.99-6.79 (m, 2H), 6.25 (s, 1H), 6.13 (q, J=3.2 Hz, 1H), 5.32 (dt, J=5.7, 2.2 Hz, 1H), 3.91 (s, 3H), 3.33 (s, 3H), 3.19 (ddd, J=16.6, 3.9, 2.2 Hz, 1H), 2.71-2.60 (m, 1H), 2.42 (s, 3H), 2.33 (s, 3H), 2.06 (s, 3H); MS (ESI+) m/z 568.0 (M+H)⁺.

### Example 36B

### rac-(1R,2S)-2-[2-(1,3-dimethyl-1H-pyrazol-4-yl)-6-methyl-7-oxo-6,7-dihydro-1H-pyrrolo[2,3-c]pyridin-4-yl]-1′-methylspiro[cyclopropane-1,3′-indol]-2′(1H)-one

A solution of Example 36A (9.3 mg, 0.016 mmol) and NaOH (0.164 mL, 0.164 mmol, 1M in water) in dioxane (0.64 mL) was heated to 80° C. After 18 hours the reaction was diluted with ethyl acetate and washed with a saturated aqueous NH₄Cl solution and brine. The organic phase was dried with MgSO₄, filtered, and concentrated under reduced pressure. The crude residue was then purified via reverse phase HPLC (5-95% acetonitrile/water with 0.1% trifluoroacetic acid) to yield the title compound (5.6 mg, 0.014 mmol, 83% yield) as solid. ¹H NMR (400 MHz, DMSO-d₆) δ 11.75 (d, J=2.2 Hz, 1H), 8.05 (s, 1H), 7.29 (td, J=7.7, 1.3 Hz, 1H), 7.13-7.03 (m, 2H), 6.91 (td, J=7.6, 1.1 Hz, 1H), 6.36 (d, J=2.2 Hz, 1H), 5.98 (td, J=3.3, 2.1 Hz, 1H), 5.03 (td, J=3.6, 1.9 Hz, 1H), 3.74 (s, 3H), 3.20 (s, 3H), 2.93-2.87 (m, 1H), 2.52 (dd, J=3.5, 1.9 Hz, 1H), 2.29 (s, 3H), 2.18 (s, 3H), MS (ESI−) m/z 412.0 (M−H)⁻.

### Example 37

### rac-(1R,2S)-1′-methyl-2-[6-methyl-7-oxo-2-(1H-pyrazol-3-yl)-6,7-dihydro-1H-pyrrolo[2,3-c]pyridin-4-yl]spiro[cyclopropane-1,3′-indol]-2′(1H)-one

### Example 37A

### rac-(1R,2S)-1′-methyl-2-[6-methyl-1-(4-methylbenzene-1-sulfonyl)-7-oxo-2-(1H-pyrazol-3-yl)-6,7-dihydro-1H-pyrrolo[2,3-c]pyridin-4-yl]spiro[cyclopropane-1,3′-indol]-2′(1H)-one

To a round bottom flask containing a stir bar was added Example 31B (250 mg, 0.417 mmol), 3-(4,4,5,5-tetramethyl-1,3,2-dioxaborolan-2-yl)-1H-pyrazole (121 mg, 0.626 mmol), PdCl₂(dppf)-CH₂Cl₂ adduct (17.03 mg, 0.021 mmol), and sodium carbonate (111 mg, 1.043 mmol). The flask was evacuated and backfilled three times with nitrogen and then charged with tetrahydrofuran (4.0 mL) and water (1.0 mL). The resulting mixture was stirred at 70° C. for 16 hours. The solution was poured into ethyl acetate and the organic fraction was washed with water and concentrated. Purification of the residue via flash chromatography (30-80% ethyl acetate/petroleumether) afforded the title compound (69 mg, 0.121 mmol, 29.1% yield). MS (ESI+) m/z 565.2 (M+H)⁺.

### Example 37B

### rac-(1R,2S)-1′-methyl-2-[6-methyl-7-oxo-2-(1H-pyrazol-3-yl)-6,7-dihydro-1H-pyrrolo[2,3-c]pyridin-4-yl]spiro[cyclopropane-1,3′-indol]-2′(1H)-one

The title compound was prepared according to the procedure used for the preparation of Example 39A, substituting Example 37A for Example 31B. Purification by reverse-phase HPLC provided the title compound and Example 38. ¹H NMR (400 MHz, CDCl₃) δ 7.65 (s, 1H), 7.61 (s, 1H), 7.18-7.08 (m, J=7.1 Hz, 2H), 6.96 (d, J=7.8 Hz, 1H), 6.72 (t, 1H), 6.60 (s, 1H), 6.38-6.27 (m,2H), 3.70 (s, 3H), 3.40 (s, 3H), 3.17 (t, J=8.3 Hz, 1H), 2.22 (dd, J=8.9, 4.6 Hz, 1H), 2.16-2.09 (m, J=7.8, 4.7 Hz, 1H), 1.29 (s, 1H). MS (ESI+) m/z 386.2 (M+H)⁺.

### Example 38

### rac-(1R,2R)-1′-methyl-2-[6-methyl-7-oxo-2-(1H-pyrazol-3-yl)-6,7-dihydro-1H-pyrrolo[2,3-c]pyridin-4-yl]spiro[cyclopropane-1,3′-indol]-2′(1H)-one

The title compound was isolated from the reverse-phase HPLC purification as described in Example 37B. ¹H NMR (400 MHz, CD₃OD) δ 7.57 (d, J=2.3 Hz, 1H), 7.47-7.45 (m, 1H), 7.35 (t, J=7.1 Hz, 1H), 7.16 (t, J=7.6 Hz, 1H), 7.09 (d, J=8.8 Hz, 2H), 7.00 (d, J=7.9 Hz, 1H), 6.55 (d, J=2.2 Hz, 1H), 6.19 (s, 1H), 4.00 (s, 1H), 3.70 (s, 3H), 3.15 (s, 3H), 2.32 (dd, J=8.4, 4.9 Hz, 1H), 2.23-2.15 (m, 1H), 2.02 (s, 1H), 1.62(s, 1H).MS (ESI+). m/z 386.2 (M+H)⁺.

### Example 39

### rac-(1R,2S)-1′-methyl-2-[6-methyl-2-(3-methyl-1,2-oxazol-5-yl)-7-oxo-6,7-dihydro-1H-pyrrolo[2,3-c]pyridin-4-yl]spiro[cyclopropane-1,3′-indol]-2′(1H)-one

### Example 39A

### rac-(1R,2S)-2-(2-iodo-6-methyl-7-oxo-6,7-dihydro-1H-pyrrolo[2,3-c]pyridin-4-yl)-1′-methylspiro[cyclopropane-1,3′-indol]-2′(1H)-one

To a mixture of Example 31B (300 mg, 0.500 mmol) in dioxane (20 mL) and water (5 mL) was added NaOH (200 mg, 5.00 mmol). The resulting mixture was stirred at about 60° C. for about 2 hours. The reaction mixture was diluted with ethyl acetate (about 50 mL), washed with water and brine, dried over sodium sulfate, filtered and concentrated. The crude product was triturated with ethyl acetate to give the title compound (200 mg, 0.427 mmol, 85% yield) as solid. MS (ESI+) m/z 446.1 (M+H)⁺.

### Example 39B

### rac-(1R,2S)-1′-methyl-2-[6-methyl-2-(3-methyl-1,2-oxazol-5-yl)-7-oxo-6,7-dihydro-1H-pyrrolo[2,3-c]pyridin-4-yl]spiro[cyclopropane-1,3′-indol]-2′(1H)-one

A mixture of Example 39A (140 mg, 0.314 mmol), (3-methylisoxazol-5-yl)boronic acid (80 mg, 0.629 mmol), Pd(Ph₃P)₄ (363 mg, 0.314 mmol) and sodium bicarbonate (26.4 mg, 0.314 mmol) in N,N-dimethylformamide (2 mL) and water (1 mL) was heated at 110° C. under microwave heating conditions for 2 hours. The reaction mixture was diluted with water and extracted with ethyl acetate (3 x 20 mL). The combined organic layers were washed with water and brine, dried with sodium sulfate, filtered and concentrated. The residue was purified by reverse-phase HPLC to provide the title compound (1.8 mg, 4.27 μmol, 1.358% yield) and Example 40. ¹H NMR (400 MHz, CD₃OD+CDCl₃) δ 7.31 (d, J=1.3 Hz, 1H), 7.15-7.09 (m, 1H), 6.99 (d, J=7.8 Hz, 1H), 6.69 (t, J=7.6 Hz, 1H), 6.56 (s, 1H), 6.35 (d, J=7.2 Hz, 1H), 6.29 (s, 1H), 3.69 (s, 3H), 3.39 (s, 3H), 3.10 (t, J=9.0 Hz, 1H), 2.29 (s, 3H), 2.23-2.16 (m, 2H). MS (ESI+) m/z 401 (M+H)⁺.

### Example 40

### rac-(1R,2R)-1′-methyl-2-[6-methyl-2-(3-methyl-1,2-oxazol-5-yl)-7-oxo-6,7-dihydro-1H-pyrrolo[2,3-c]pyridin-4-yl]spiro[cyclopropane-1,3′-indol]-2′(1H)-one

Example 40 was isolated from the reverse-phase HPLC purification as described in Example 39B. ¹H NMR (400 MHz, CDCl₃+CD₃OD) δ 7.31 (s, 1H), 7.11 (s, 1H), 7.02 (s, 2H), 6.94 (d, J=8.0 Hz, 1H), 6.39 (s, 1H), 6.21 (s, 1H), 3.63 (s, 3H), 3.09 (s, 3H), 3.03 (s, 1H), 2.27 (s, 3H), 2.21-2.06 (m, 2H). MS (ESI+) m/z 401 (M+H)⁺.

### Example 41

### rac-(1R,2S)-1′-methyl-2-[6-methyl-2-(1-methyl-1H-imidazol-2-yl)-7-oxo-6,7-dihydro-1H-pyrrolo[2,3-c]pyridin-4-yl]spiro[cyclopropane-1,3′-indol]-2′(1H)-one

To a round bottom flask with stir bar was added Example 39A (150 mg, 0.337 mmol), 1-methyl-2-(tributylstannyl)-1H-imidazole (125 mg, 0.337 mmol), bis(triphenylphosphine)palladium(II) chloride (11.82 mg, 0.017 mmol), copper(I) iodide (3.21 mg, 0.017 mmol) and lithium chloride (35.7 mg, 0.842 mmol). The flask was evacuated and backfilled three times with nitrogen and then charged with N,N-dimethylformamide (5 mL). The resulting mixture was heated at 100° C. using a Biotage microwave reactor for 2 hours. The mixture was partitioned between ethyl acetate and water. The organic layer was separated, dried over sodium sulfate and concentrated. The residue was purified by HPLC to give the title compound (12.2 mg, 0.031 mmol, 9.07% yield) as solid. ¹H NMR (400 MHz, CDCl₃) δ 12.01 (s, 1H), 7.44 (d, J=18.5 Hz, 2H), 7.11 (d, J=0.9 Hz, 1H), 6.94 (d, J=1.2 Hz, 1H), 6.85 (d, J=7.8 Hz, 1H), 6.67 (s, 1H),6.24 (d, J=7.4 Hz, 1H), 5.89 (d, J=1.5 Hz, 1H), 3.65 (s, 3H), 3.52 (s, 3H), 3.36 (s, 3H), 3.17 (s, 1H), 2.25 (s, 1H), 1.97-1.93 (m, 1H). MS (ESI+) m/z 400.1 (M+H)⁺.

### Example 42

### rac-(1R,2S)-1′-methyl-2-[6-methyl-2-(1,3-oxazol-2-yl)-7-oxo-6,7-dihydro-1H-pyrrolo[2,3-c]pyridin-4-yl]spiro[cyclopropane-1,3′-indol]-2′(1H)-one

Example 42 was prepared according to the procedure used for the preparation of Example 41, substituting 2-(tributylstannyl)oxazole for 1-methyl-2-(tributylstannyl)-1H-imidazole. ¹H NMR (400 MHz, CDCl₃) δ 7.70 (s, 1H), 7.13 (t, 1H), 6.93-6.85 (m, 2H), 6.70 (t, 1H), 6.64 (s, 1H), 6.25 (d, J=7.5 Hz, 1H), 3.70 (s, 3H), 3.38 (s,3H), 3.24-3.16 (m, 1H), 2.29-2.18 (m, 1H), 1.95-1.90 (m, 1H). MS (ESI+) m/z 387.1 (M+H)⁺.

### Example 43

### rac-(1R,2S)-1′-methyl-2-[6-methyl-2-(1-methyl-1H-imidazol-4-yl)-7-oxo-6,7-dihydro-1H-pyrrolo[2,3-c]pyridin-4-yl]spiro[cyclopropane-1,3′-indol]-2′(1H)-one

Example 43 was prepared according to the procedure used for the preparation of Example 41, substituting 1-methyl-4-(tributylstannyl)-1H-imidazole for 1-methyl-2-(tributylstannyl)-1H-imidazole. ¹H NMR (400 MHz, CDCl₃) δ 7.33 (s, 1H), 7.14 (s, 1H), 6.99 (s, 1H), 6.77 (s, 2H), 6.57 (s, 1H), 6.15 (s, 2H), 3.59 (d, J=6.2 Hz, 3H), 3.51 (d, J=6.1 Hz, 3H),3.23 (d, J=6.0 Hz, 3H), 3.06-2.97 (m, 1H), 2.10-2.04 (m, 1H), 1.91-1.82 (m, 1H). MS (ESI+) m/z 400.2 (M+H)⁺.

### Example 44

### rac-(1R,2S)-1′-methyl-2-[6-methyl-2-(1-methyl-1H-imidazol-5-yl)-7-oxo-6,7-dihydro-1H-pyrrolo[2,3-c]pyridin-4-yl]spiro[cyclopropane-1,3′-indol]-2′(1H)-one

Example 43 was prepared according to the procedure used for the preparation of Example 41, substituting 1-methyl-5-(tributylstannyl)-1H-imidazole for 1-methyl-2-(tributylstannyl)-1H-imidazole. ¹H NMR (400 MHz, CDCl₃) δ 12.26 (s, 1H), 7.46 (d, J=8.4 Hz, 2H), 7.10 (dd, J=7.7, 1.0 Hz, 1H), 6.94 (d, J=1.3 Hz, 1H), 6.85 (d, J=7.7 Hz, 1H), 6.67(td, J=7.6, 0.7 Hz, 1H), 6.24 (d, J=7.1 Hz, 1H), 5.89 (s, 1H), 3.65 (s, 3H), 3.52 (s, 3H), 3.36 (s, 3H), 3.17 (s, 1H), 2.25 (dd, J=8.9, 4.4 Hz, 1H), 1.94 (dd, J=7.7, 4.4 Hz, 1H). MS (ESI+) m/z 400.1 (M+H)⁺.

### Example 45

### rac-(1R,2S)-1′-methyl-2-[6-methyl-7-oxo-2-(pyridin-2-yl)-6,7-dihydro-1H-pyrrolo[2,3-c]pyridin-4-yl]spiro[cyclopropane-1,3′-indol]-2′(1H)-one

### Example 45A

### rac-(1R,2S)-1′-methyl-2-[6-methyl-1-(4-methylbenzene-1-sulfonyl)-7-oxo-2-(pyridin-2-yl)-6,7-dihydro-1H-pyrrolo[2,3-c]pyridin-4-yl]spiro[cyclopropane-1,3′-indol]-2′(1H)-one

Example 45A was prepared according to the procedure used for the preparation of Example 41, substituting Example 31B for Example 39A and 2-(tributylstannyl)pyridine for 1-methyl-2-(tributylstannyl)-1H-imidazole. MS (ESI+) m/z 401 (M+H)⁺.

### Example 45B

### rac-(1R,2S)-1′-methyl-2-[6-methyl-7-oxo-2-(pyridin-2-yl)-6,7-dihydro-1H-pyrrolo[2,3-c]pyridin-4-yl]spiro[cyclopropane-1,3′-indol]-2′(1H)-one

A solution of Example 45A (40 mg, 0.073 mmol) in dioxane (4 mL) and water (1 mL) was treated with LiOH (17.40 mg, 0.726 mmol) and stirred at ambient temperature for 4 days while monitoring by LCMS. The mixture was diluted with ethyl acetate (20 mL) and extracted with pH7 buffer solution. The organic phase was dried over sodium sulfate, filtered and concentrated. The residue was purified by reverse-phase HPLC to give the title compound (8.8 mg, 0.022 mmol, 30.6% yield). ¹H NMR (400 MHz, CD₃OD) δ 8.56 (d, J=4.2 Hz, 1H), 7.77 (td, J=7.6, 1.3 Hz, 1H), 7.67 (d, J=8.0 Hz, 1H), 7.25 (dd, J=7.2, 5.1 Hz, 1H), 7.13 (ddd, J=7.8, 7.2, 1.5 Hz, 2H), 6.96 (d, J=7.8 Hz, 1H), 6.78-6.68 (m, 1H), 6.53 (s, 1H), 6.35 (d, J=7.5 Hz, 1H), 3.69 (d, J=5.0 Hz, 3H), 3.41 (s, 3H), 3.23-3.16 (m,1H), 2.25 (dd, J=9.0, 4.7 Hz, 1H), 2.11 (dd, J=8.6, 3.8 Hz, 1H). MS (ESI+) m/z 397.1 (M+H)⁺.

### Example 46

### rac-(1R,2S)-1′-methyl-2-[6-methyl-2-(4-methylpyridin-2-yl)-7-oxo-6,7-dihydro-1H-pyrrolo[2,3-c]pyridin-4-yl]spiro[cyclopropane-1,3′-indol]-2′(1H)-one

Example 46 was prepared according to the procedure used for the preparation of Example 45, substituting 4-methyl-2-(tributylstannyl)pyridine for 2-(tributylstannyl)pyridine. ¹H NMR (400 MHz, CDCl₃) δ 8.41 (d, J=5.0 Hz, 1H), 7.46 (s, 1H), 7.29 (d, J=7.6 Hz, 2H), 7.00 (d, J=5.1 Hz, 1H), 6.94-6.85 (m, J=17.8, 7.6 Hz, 2H), 6.79(s, 1H), 5.99-5.93 (m, J=2.1 Hz, 1H), 5.35 (s, 1H), 3.31 (s, 3H), 3.16 (d, J=12.0 Hz, 1H), 2.63 (d, J=14.5 Hz, 1H), 2.39 (d, J=3.3 Hz, 6H), 2.25-2.18 (m,1H). MS (ESI+) m/z 411.1 (M+H)⁺.

### Example 47

### rac-(1R,2S)-1′-methyl-2-[6-methyl-7-oxo-2-(pyridin-3-yl)-6,7-dihydro-1H-pyrrolo[2,3-c]pyridin-4-yl]spiro[cyclopropane-1,3′-indol]-2′(1H)-one

### Example 47A

### rac-(1R,2S)-1′-methyl-2-[6-methyl-1-(4-methylbenzene-1-sulfonyl)-7-oxo-2-(pyridin-3-yl)-6,7-dihydro-1H-pyrrolo[2,3-c]pyridin-4-yl]spiro[cyclopropane-1,3′-indol]-2′(1H)-one

A 5 mL microwave tube with stir bar was charged with Example 31B (79.5 mg, 0.133 mmol), 3-(4,4,5,5-tetramethyl-1,3,2-dioxaborolan-2-yl)pyridine (47.7 mg, 0.233 mmol), cesium carbonate (122 mg, 0.374 mmol) and [(1,3,5,7-tetramethyl-6-phenyl-2,4,6-trioxa-6-phosphaadamantane)-2-(2′-amino-1,1′-biphenyl)]palladium(II) methanesulfonate (5.8 mg, 8.76 μmol). The vessel was sealed and purged with nitrogen for 15 minutes, followed by addition of a degassed mixture of tetrahydrofuran (2 mL) and water (0.500 mL). The mixture was heated at 60° C. for 5 hours. The mixture was diluted with 40 mL of ethyl acetate and washed with saturated aqueous sodium chloride. The organic phase was dried over sodium sulfate, filtered and concentrated. After filtration and solvent removal, the residue was purified by flash chromatography (4 g silica cartridge, eluting with 10-100% 3:1 ethyl acetate:ethanol/heptanes) to provide the title compound as solid (49 mg, 67%).

### Example 47B

### rac-(1R,2S)-1′-methyl-2-[6-methyl-7-oxo-2-(pyridin-3-yl)-6,7-dihydro-1H-pyrrolo[2,3-c]pyridin-4-yl]spiro[cyclopropane-1,3′-indol]-2′(1H)-one

Example 47B was prepared according to the procedure used for the preparation of Example 45B, substituting Example 47A for Example 45A. ¹H NMR (400 MHz, DMSO-d₆) δ 12.41 (s, 1H), 8.95 (dd, J=2.4, 0.8 Hz, 1H), 8.41 (dd, J=4.8, 1.6 Hz, 1H), 8.14 (ddd, J=8.0, 2.4, 1.6 Hz, 1H), 7.39 (s, 1H), 7.36 (m, 1H), 7.05 (td, J=7.7, 1.2 Hz, 1H), 6.95 (d, J=7.7 Hz, 1H), 6.67 (td, J=7.5, 1.1 Hz, 1H), 6.45-6.37 (m, 2H), 3.55 (s, 3H), 3.24 (s, 3H), 2.94 (m, 1H), 2.25 (dd, J=7.8, 4.7 Hz, 1H), 1.98 (dd, J=9.0, 4.6 Hz, 1H). MS (ESI+) m/z 397.3 (M+H)⁺.

### Example 48

### (1S,2R)-1′-methyl-2-[6-methyl-7-oxo-2-(pyridin-3-yl)-6,7-dihydro-1H-pyrrolo[2,3-c]pyridin-4-yl]spiro[cyclopropane-1,3′-indol]-2′(1H)-one

### Example 48A

### (1S,2R)-2-[2-iodo-6-methyl-1-(4-methylbenzene-1-sulfonyl)-7-oxo-6,7-dihydro-1H-pyrrolo[2,3-c]pyridin-4-yl]-1′-methylspiro[cyclopropane-1,3′-indol]-2′(1H)-one

The enantiomers of Example 31B were separated by preparative chiral SFC chromatography under the following conditions: Instrument: SFC-200 (Thar, Waters); Column: Chiralcel OD 20×250 mm, 5 μm (Diacel); Column temperature: 35° C.; Mobile phase: 65:35 CO₂/methanol (0.2% NH₄OH); Flow rate: 160 g/min; Back pressure: 100 bar; Detection wavelength: 214 nm; Cycle time: 5.3 min; Sample solution: 12 g dissolved in 650 mL methanol; Injection volume: 2.0 mL. Separation provided 4.625 g (retention time 2.8 minutes under the following conditions: Column: EnantioPak OD 4.6 x 100 mm, 5 μm; Column temperature: 39.9° C.; Mobile phase: 70:30 CO₂/methanol (0.2% Methanol Ammonia); CO₂ flow rate: 2.8 g/min; Back pressure: 118 bar; Detection wavelength: 230 nm; 38% yield; 97.6% ee) of Example 48A. ^{I}IINNIR (400 MHz, CDCl₃) δ 8.22 (d, J=8.4 Hz, 2H), 7.35 (d, J=8.2 Hz, 2H), 7.20 (t, J=1.0 Hz, 1H), 6.94-6.86 (m, J=4.5, 3.2 Hz, 2H), 6.75 (t, 1H), 6.48 (s, 1H), 6.21 (d, J=7.0 Hz, 1H), 3.53 (s, 3H), 3.36 (s, 3H), 2.96 (t, 1H), 2.42 (s, 3H), 2.19 (dd, J=9.0, 4.5 Hz, 1H), 1.78 (dd, J=7.7, 4.5 Hz, 1H). MS (ESI+) m/z 600.1 (M+H)⁺.

### Example 48B

### (1R,2S)-2-[2-iodo-6-methyl-1-(4-methylbenzene-1-sulfonyl)-7-oxo-6,7-dihydro-1H-pyrrolo[2,3-c]pyridin-4-yl]-1′-methylspiro[cyclopropane-1,3′-indol]-2′(1H)-one

The title compound (retention time 3.6 minutes under the following conditions: Column: EnantioPak OD 4.6×100 mm, 5 μm; Column temperature: 39.9° C.; Mobile phase: 70:30 CO₂/methanol (0.2% Methanol Ammonia); CO₂ flow rate: 2.8 g/min; Back pressure: 118 bar; Detection wavelength: 230 nm; 4.76 g, 39% yield, 97.1% ee) was isolated by the preparative chiral SFC chromatography as described in Example 48A. ¹H NMR (400 MHz, CDCl₃) δ 8.22 (d, J=8.4 Hz, 4H), 7.35 (d, J=8.1 Hz, 4H), 7.19 (dd, J=7.7, 1.0 Hz, 2H), 6.95-6.89 (m, J=4.5, 3.2 Hz, 4H), 6.75 (t, J=7.6 Hz, 2H), 6.48 (s, 2H), 6.21 (d, J=7.4 Hz, 2H), 5.30 (s, 1H), 3.53 (s, 6H), 3.37 (s, 6H), 2.97 (t, 2H), 2.43 (s, 6H), 2.24-2.15 (m, J=4.5 Hz, 2H), 1.86-1.75 (m, 2H). MS (ESI+) m/z 600.1 (M+H)⁺.

### Example 48C

### (1S,2R)-1′-methyl-2-[6-methyl-1-(4-methylbenzene-1-sulfonyl)-7-oxo-2-(pyridin-3-yl)-6,7-dihydro-1H-pyrrolo[2,3-c]pyridin-4-yl]spiro[cyclopropane-1,3′-indol]-2′(1H)-one

A 5 mL microwave tube with stirbar was charged with Example 48A (0.05 g, 0.083 mmol), 3-(tributylstannyl)pyridine (0.045 mL, 0.142 mmol) and bis(triphenylphosphine)palladium(II) chloride (5.85 mg, 8.34 μmol) and sealed. The vessel was purged with nitrogen for 30 minutes, followed by addition of nitrogen-sparged N,N-dimethyl formamide (0.8 mL). The mixture was heated in a Biotage microwave reactor at 120° C. for one hour, then cooled to room temperature and partitioned between ethyl acetate and water. The organic layer was washed with brine, treated with 3-mercaptopropyl-functionalized silica gel for 20 minutes, dried over anhydrous magnesium sulfate, filtered through a plug of diatomaceous earth and concentrated. The residue was purified by flash chromatography on a 25 g silica gel column eluting with 0 to 100% of a 3:1 mixture of ethyl acetate/ethanol in heptanes to provide the title compound (0.0152 g, 33% yield).

### Example 48D

### (1S,2R)-1′-methyl-2-[6-methyl-7-oxo-2-(pyridin-3-yl)-6,7-dihydro-1H-pyrrolo[2,3-c]pyridin-4-yl]spiro[cyclopropane-1,3′-indol]-2′(1H)-one

A flask charged with Example 48C (0.038 g, 0.069 mmol), lithium hydroxide-monohydrate (0.017 g, 0.413 mmol), 1,4-dioxane (1 mL) and water (0.25 mL) was heated at 80° C. for 4.5 hours. The mixture was cooled to ambient temperature and concentrated to dryness. The residue was taken up in 3 mL of 1:1 DMSO/methanol plus several drops of trifluoroacetic acid. A minimal amount of white solid was removed by syringe filtration. The filtrate was purified by reverse phase HPLC (C18, 10-70% acetonitrile/water (0.1% trifluoroacetic acid)) and the fractions dried by lyophilization to provide the trifluoroacetic acid salt of the title compound (0.039 g, 111% yield, 55.4 ee). ¹H NMR (400 MHz, DMSO-d₆) δ 12.52 (s, 1H), 9.08 (s, 1H), 8.52 (d, J=4.9 Hz, 1H), 8.38 (d, J=8.1 Hz, 1H), 7.54 (dd, J=7.9, 5.1 Hz, 1H), 7.44 (m, 1H), 7.08 (m, 1H), 6.98 (d, J=7.6 Hz, 1H), 6.70 (m, 1H), 6.53 (d, J=2.2 Hz, 1H), 6.46 (d, J=7.4 Hz, 1H), 3.59 (s, 3H), 3.28 (s, 3H), 2.97 (t, J=8.4 Hz, 1H), 2.29 (dd, J=7.8, 4.7 Hz, 1H), 2.01 (dd, J=8.9, 4.6 Hz, 1H). MS (ESI+) m/z 397.3 (M+H)⁺.

### Example 49

### (1R,2S)-1′-methyl-2-[6-methyl-7-oxo-2-(pyridin-3-yl)-6,7-dihydro-1H-pyrrolo[2,3-c]pyridin-4-yl]spiro[cyclopropane-1,3′-indol]-2′(1H)-one

### Example 49A

### (1R,2S)-1′-methyl-2-[6-methyl-1-(4-methylbenzene-1-sulfonyl)-7-oxo-2-(pyridin-3-yl)-6,7-dihydro-1H-pyrrolo[2,3-c]pyridin-4-yl]spiro[cyclopropane-1,3′-indol]-2′(1H)-one

To a solution of Example 48B (0.03 g, 0.05 mmol) in N,N-dimethyl formamide (0.5 mL) was added 3-(tributylstannyl)pyridine (0.027 mL, 0.085 mmol), cuprous iodide (1.9 mg, 10.0 μmol) and bis(triphenylphosphine)palladium(II) dichloride (3.5 mg, 5.0 μmol). The mixture was heated in a Biotage microwave reactor at 120° C. for 1 hour. The reaction mixture was partitioned between ethyl acetate and water, washed twice with brine, dried over anhydrous sodium sulfate, treated with functionalized Silica Met Thiol, filtered and concentrated. The residue was purified on silica gel eluting with 1-6% methanol in dichloromethane to afford the title compound (0.013 g, 48% yield). ¹H NMR (501 MHz, DMSO-d₆) δ 8.55 (dd, J=5.0, 1.6 Hz, 1H), 8.43 (d, J=2.2 Hz, 1H), 7.75 (m, 2H), 7.69 (m, 2H), 7.40 (ddd, J=7.9, 4.9, 0.9 Hz, 1H), 7.37 (m, 2H), 7.15 (td, J=7.7, 1.2 Hz, 1H), 7.00 (d, J=7.8 Hz, 1H), 6.71 (td, J=7.5, 1.0 Hz, 1H), 6.34 (dd, J=7.5, 1.1 Hz, 1H), 6.19 (s, 1H), 3.46 (s, 3H), 3.20 (s, 3H), 2.88 (ddd, J=9.0, 7.8, 1.3 Hz, 1H), 2.38 (s, 3H), 2.20 (dd, J=7.7, 4.7 Hz, 1H), 1.99 (dd, J=9.0, 4.7 Hz, 1H). MS (ESI+) m/z 551.2 (M+H)⁺.

### Example 49B

### (1R,2S)-1′-methyl-2-[6-methyl-7-oxo-2-(pyridin-3-yl)-6,7-dihydro-1H-pyrrolo[2,3-c]pyridin-4-yl]spiro[cyclopropane-1,3′-indol]-2′(1H)-one

A solution of Example 49A (0.045 g, 0.082 mmol) in 1,4-dioxane (0.63 mL) and aqueous sodium hydroxide (0.2 mL, 0.2 mmol, 1 M) was heated at 80° C. for 3 hours and then stirred at ambient temperature overnight. The reaction mixture was diluted with ethyl acetate, washed with saturated sodium bicarbonate solution and brine, dried over anhydrous sodium sulfate, filtered and concentrated. The crude product was purified by chromatography on silica gel eluting with 1-6% methanol in dichloromethane to give the title compound (0.012 g, 96% yield, 73.4% ee). ¹H NMR (400 MHz, DMSO-d₆) δ 12.42 (s, 1H), 8.97 (dd, J=2.4, 0.8 Hz, 1H), 8.43 (dd, J=4.7, 1.6 Hz, 1H), 8.17 (ddd, J=8.0, 2.3, 1.6 Hz, 1H), 7.41 (d, J=1.3 Hz, 1H), 7.36 (ddd, J=8.1, 4.8, 0.8 Hz, 1H), 7.07 (td, J=7.7, 1.2 Hz, 1H), 6.97 (d, J=7.7 Hz, 1H), 6.68 (td, J=7.5, 1.1 Hz, 1H), 6.44 (dd, J=7.5, 1.2 Hz, 1H), 6.41 (d, J=2.2 Hz, 1H), 3.57 (s, 3H), 3.26 (s, 3H), 2.95 (m, 1H), 2.27 (dd, J=7.9, 4.6 Hz, 1H), 1.99 (dd, J=9.0, 4.6 Hz, 1H). MS (ESI+) m/z 397.3 (M+H)⁺.

### Example 50

### rac-(1R,2S)-1′-methyl-2-[6-methyl-7-oxo-2-(pyridin-4-yl)-6,7-dihydro-1H-pyrrolo[2,3-c]pyridin-4-yl]spiro[cyclopropane-1,3′-indol]-2′(1H)-one

Example 50 was prepared according to the procedure used for the preparation of Example 41, substituting 4-(tributylstannyl)pyridine for 1-methyl-2-(tributylstannyl)-1H-imidazole. ¹H NMR (400 MHz, CDCl₃) δ 12.25 (s, 1H), 8.53 (d, J=5.4 Hz, 2H), 7.68 (d, J=6.1 Hz, 2H), 7.05 (td, J=7.7, 1.1 Hz, 1H), 6.83 (dd, J=10.4, 4.5 Hz, 2H), 6.68-6.52 (m, 1H), 6.40 (d, J=2.2 Hz, 1H), 6.20 (d, J=7.1 Hz, 1H), 3.64 (s, 3H), 3.32 (s, 3H), 3.20-3.08 (m, 1H), 2.21-2.16 (m, J=4.5 Hz,1H), 1.91-1.86 (m, 1H). MS (ESI+) m/z 397.0 (M+H)⁺.

### Example 51

### rac-(1R,2S)-1′-methyl-2-{6-methyl-7-oxo-2-[2-(trifluoromethyl)pyridin-4-yl]-6,7-dihydro-1H-pyrrolo[2,3-c]pyridin-4-yl}spiro[cyclopropane-1,3′-indol]-2′(1H)-one

### Example 51A

### rac-(1R,2S)-1′-methyl-2-{6-methyl-1-(4-methylbenzene-1-sulfonyl)-7-oxo-2-[2-(trifluoromethyl)pyridin-4-yl]-6,7-dihydro-1H-pyrrolo[2,3-c]pyridin-4-yl}spiro[cyclopropane-1,3′-indol]-2′(1H)-one

Example 31B (41.9 mg, 0.070 mmol),(2-(trifluoromethyl)pyridin-4-yl)boronic acid (20 mg, 0.105 mmol, CombiBlocks), tris(dibenzylideneacetone)dipalladium(0) (1.279 mg, 1.397 μmol), 1,3,5,7-tetramethyl-6-phenyl-2,4,8-trioxa-6-phosphaadamantane (0.980 mg, 3.35 μmol), and potassium phosphate tribasic (37.1 mg, 0.175 mmol) were combined in a 4 mL vial with stir bar and septum cap. The vial was evacuated and backfilled with nitrogen three times before being charged with dioxane (1.1 mL) and water (0.28 mL). The resulting mixture was then heated at 60° C. for 18 hours. The reaction was diluted with ethyl acetate and washed with water and brine. The organic phase was then dried with MgSO₄, filtered, and concentrated under reduced pressure. The crude residue was purified via flash chromatography (ISCO Combiflash, 50-100% ethyl acetate/heptanes, 12 g Redisep Gold silica column) to yield the title compound (27.7 mg, 0.045 mmol, 64.1% yield) as solid. ¹M NMR (400 MHz, CDCl₃) δ 8.73 (d, J=4.9 Hz, 1H), 8.03-7.97 (m, 2H), 7.44-7.38 (m, 2H), 7.37-7.31 (m, 2H), 7.22 (td, J=7.8, 1.2 Hz, 1H), 7.10 (d, J=1.4 Hz, 1H), 6.99-6.87 (m, 1H), 6.76 (td, J=7.6, 1.0 Hz, 1H), 6.22 (dt, J=7.5, 0.7 Hz, 1H), 6.03 (s, 1H), 3.62 (s, 3H), 3.35 (s, 3H), 3.03 (ddd, J=9.0, 7.6, 1.5 Hz, 1H), 2.46 (s, 3H), 2.25 (dd, J=8.9, 4.6 Hz, 1H), 1.84 (dd, J=7.7, 4.6 Hz, 1H); MS (ESI+) m/z 619.2 (M+H)⁺.

### Example 51B

### rac-(1R,2S)-1′-methyl-2-{6-methyl-7-oxo-2-[2-(trifluoromethyl)pyridin-4-yl]-6,7-dihydro-1H-pyrrolo[2,3-c]pyridin-4-yl}spiro[cyclopropane-1,3′-indol]-2′(1H)-one

Aqueous NaOH (0.113 mL, 0.113 mmol, 1M in water) was added to a solution of Example 51A (28 mg, 0.045 mmol) in dioxane (0.35 mL) and the resulting mixture was heated to 80° C. After 1 hour the reaction was cooled to room temperature and diluted with ethyl acetate. The reaction was then washed with a saturated aqueous NaHCO₃ solution and brine, dried with MgSO₄, filtered, and concentrated under reduced pressure. The crude residue was then purified via flash chromatography (ISCO Combiflash, 2-6% methanol/dichloromethane, 12 g Redisep Gold silica column) to yield the title compound (13.6 mg, 0.029 mmol, 64.7% yield) as solid. ¹H NMR (501 MHz, DMSO-d₆) δ 12.76 (s, 1H), 8.65 (d, J=5.3 Hz, 1H), 8.52-8.38 (m, 1H), 8.10 (dd, J=5.2, 1.7 Hz, 1H), 7.43 (d, J=1.2 Hz, 1H), 7.06 (td, J=7.7, 1.2 Hz, 1H), 6.97 (d, J=7.8 Hz, 1H), 6.83 (s, 1H), 6.68 (td, J=7.5, 1.0 Hz, 1H), 6.46-6.41 (m, 1H), 3.58 (s, 3H), 3.26 (s, 3H), 2.97 (ddd, J=9.0, 7.8, 1.2 Hz, 1H), 2.28 (dd, J=7.8, 4.7 Hz, 1H), 1.99 (dt, J=9.2, 4.6 Hz, 1H); MS (ESI−) m/z 463.0 (M−H)⁻.

### Example 52

### (1S,2R)-1′-methyl-2-{6-methyl-7-oxo-2-[2-(trifluoromethyl)pyridin-4-yl]-6,7-dihydro-1H-pyrrolo[2,3-c]pyridin-4-yl}spiro[cyclopropane-1,3′-indol]-2′(1H)-one

The enantiomers of Example 51B were separated by preparative chiral SFC chromatography under the following conditions: Column: ChiralPak IB 21×250 mm, 5 μm; Column temperature: 34° C.; Mobile phase: 3:1 CO₂/methanol; Flow rate: 69 g/min; Back pressure: 100 bar; Detection wavelength: 220 nm; Cycle time: 4.5 min; Sample solution: 195 mg dissolved in 19.5 mL 1:1 DMSO/methanol; Injection volume: 0.5 mL. Separation provided 0.0353 g (18% yield; 99% ee) of the title compound. ¹H NMR (400 MHz, DMSO-d₆) δ 12.80 (s, 1H), 8.68 (d, J=5.2 Hz, 1H), 8.46 (s, 1H), 8.13 (m, 1H), 7.46 (s, 1H), 7.08 (m, 1H), 6.99 (d, J=7.7 Hz, 1H), 6.86 (s, 1H), 6.70 (t, J=7.4 Hz, 1H), 6.46 (d, J=7.2 Hz, 1H), 3.60 (s, 3H), 3.29 (s, 3H), 2.99 (t, J=8.2 Hz, 1H), 2.31 (dd, J=7.8, 4.7 Hz, 1H), 2.01 (dd, J=8.9, 4.6 Hz, 1H). MS (ESI+) m/z 465.3 (M+H)⁺.

### Example 53

### (1R,2S)-1′-methyl-2-{6-methyl-7-oxo-2-[2-(trifluoromethyl)pyridin-4-yl]-6,7-dihydro-1H-pyrrolo[2,3-c]pyridin-4-yl}spiro[cyclopropane-1,3′-indol]-2′(1H)-one

The title compound (retention time 4.53 minutes, 0.0754 g, 39% yield, 90.9% ee) was isolated from the preparative chiral SFC chromatography as described in Example 52. ¹H NMR (400 MHz, DMSO-d₆) δ 12.80 (s, 1H), 8.68 (d, J=5.3 Hz, 1H), 8.46 (s, 1H), 8.13 (m, 1H), 7.46 (s, 1H), 7.08 (m, 1H), 6.99 (d, J=7.7 Hz, 1H), 6.86 (s, 1H), 6.70 (m, 1H), 6.46 (d, J=7.3 Hz, 1H), 3.60 (s, 3H), 3.29 (s, 3H), 2.99 (m, 1H), 2.31 (dd, J=7.8, 4.7 Hz, 1H), 2.02 (dd, J=8.9, 4.6 Hz, 1H). MS (ESI+) m/z 465.3 (M+H)⁺.

### Example 54

### rac-(1R,2S)-1′-methyl-2-[6-methyl-7-oxo-2-(pyrimidin-5-yl)-6,7-dihydro-1H-pyrrolo[2,3-c]pyridin-4-yl]spiro[cyclopropane-1,3′-indol]-2′(1H)-one

### Example 54A

### rac-(1R,2S)-1′-methyl-2-[6-methyl-1-(4-methylbenzene-1-sulfonyl)-7-oxo-2-(pyrimidin-5-yl)-6,7-dihydro-1H-pyrrolo[2,3-c]pyridin-4-yl]spiro[cyclopropane-1,3′-indol]-2′(1H)-one

Pyrimidine-5-boronic acid pinacol ester (30.9 mg, 0.150 mmol, Combi-Blocks), Example 31B (60 mg, 0.100 mmol), tris(dibenzylideneacetone)dipalladium(0) (1.833 mg, 2.002 μmol), 1,3,5,7-tetramethyl-6-phenyl-2,4,8-trioxa-6-phosphaadamantane (1.404 mg, 4.80 μmol), and potassium phosphate tribasic (53.1 mg, 0.250 mmol) were combined in a 4 mL vial with stir bar and septum cap. The vial was evacuated and backfilled three times with nitrogen and then charged with dioxane (834 μL) and water (167 μL). The reaction was heated to 60° C. After 2.5 hours the reaction was diluted with ethyl acetate and washed with water and brine. The organic phase was dried with MgSO₄, filtered, and concentrated under reduced pressure. The crude residue was purified via flash chromatography (ISCO Combiflash, 0-5% methanol/dichloromethane, 12 g Redisep Gold silca column) to yield the title compound (31 mg, 0.056 mmol, 56.1% yield) as solid. ¹H NMR (400 MHz, CDCl₃) δ 9.21 (d, J=3.5 Hz, 1H), 8.60 (s, 2H), 7.94-7.85 (m, 2H), 7.39-7.29 (m, 2H), 7.25-7.18 (m, 1H), 7.10 (d, J=1.5 Hz, 1H), 6.91 (dd, J=7.9, 0.9 Hz, 1H), 6.74 (td, J=7.6, 1.0 Hz, 1H), 6.21 (dt, J=7.3, 1.0 Hz, 1H), 6.00 (d, J=8.8 Hz, 1H), 3.60 (s, 3H), 3.34 (s, 3H), 3.03 (ddd, J=9.0, 7.6, 1.4 Hz, 1H), 2.44 (s, 3H), 2.23 (ddd, J=7.5, 4.7, 3.0 Hz, 1H), 1.83 (dd, J=7.6, 4.5 Hz, 1H); MS (ESI−) m/z 550.2 (M−H)⁻.

### Example 54B

### rac-(1R,2S)-1′-methyl-2-[6-methyl-7-oxo-2-(pyrimidin-5-yl)-6,7-dihydro-1H-pyrrolo[2,3-c]pyridin-4-yl]spiro[cyclopropane-1,3 ′-indol]-2′(1H)-one

A mixture of LiOH (27.4 mg, 1.142 mmol) and Example 54A (105 mg, 0.190 mmol) in dioxane (1428 μL) and water (476 μL) was heated at 100° C. for 1 hour. After cooling to room temperature, acetic acid (0.2 mL) was added to acidify the mixture and the reaction mixture was concentrated. The resulting solid was repeatedly washed with water and then dried in a vacuum oven. The crude material was recrystallized from hot N,N-dimethylformamide to yield the title compound (28 mg, 0.066 mmol, 34.4% yield) as solid. ¹H NMR (501 MHz, DMSO-d₆) δ 12.61 (s, 1H), 9.21 (s, 2H), 9.04 (s, 1H), 7.44 (d, J=1.3 Hz, 1H), 7.08 (td, J=7.7, 1.2 Hz, 1H), 7.00-6.94 (m, 1H), 6.69 (td, J=7.5, 1.0 Hz, 1H), 6.62 (s, 1H), 6.47-6.42 (m, 1H), 3.59 (s, 3H), 3.27 (s, 3H), 2.96 (ddd, J=9.2, 7.8, 1.3 Hz, 1H), 2.28 (dd, J=7.9, 4.7 Hz, 1H), 2.00 (dd, J=9.0, 4.6 Hz, 1H); MS (ESI+) m/z 398.2 (M+H)⁺.

### Example 55

### rac-(1R,2S)-1′-methyl-2-[6-methyl-7-oxo-2-(pyrimidin-4-yl)-6,7-dihydro-1H-pyrrolo[2,3-c]pyridin-4-yl]spiro[cyclopropane-1,3′-indol]-2′(1H)-one

Example 55 was prepared according to the procedure used for the preparation of Example 41, substituting 4-(tributylstannyl)pyrimidine for 1-methyl-2-(tributylstannyl)-1H-imidazole. ¹H NMR (400 MHz, CD₃OD) δ 9.08 (d, J=1.2 Hz, 1H), 8.68 (d, J=5.5 Hz, 1H), 7.78-7.73 (m, 1H), 7.29 (d, J=1.2 Hz, 1H), 7.16-7.07 (m, 1H), 6.98 (d, J=7.8 Hz, 1H), 6.74-6.65 (m, 2H), 6.37 (d, J=7.2 Hz, 1H), 3.70 (s, 3H), 3.40 (s, 3H), 3.19-3.11 (m, 1H), 2.20 (d, J=8.5 Hz, 2H). MS (ESI+) m/z 398 (M+H)⁺.

### Example 56

### rac-(1R,2S)-1′-methyl-2-[6-methyl-7-oxo-2-(pyridazin-4-yl)-6,7-dihydro-1H-pyrrolo[2,3-c]pyridin-4-yl]spiro[cyclopropane-1,3′-indol]-2′(1H)-one

Example 56 was prepared according to the procedure used for the preparation of Example 41, substituting 4-(tributylstannyl)pyridazine for 1-methyl-2-(tributylstannyl)-1H-imidazole. ¹H NMR (400 MHz, CDCl₃) δ 9.31 (s, 1H), 9.02 (s, 1H), 7.68 (d, J=2.4 Hz, 1H), 7.04 (d, J=6.7 Hz, 1H), 6.90 (s, 1H), 6.84 (d, J=7.5 Hz, 1H), 6.61(d, J=7.3 Hz, 1H), 6.46 (d, J=2.5 Hz, 1H), 6.18 (d, J=4.6 Hz, 1H), 3.59 (d, J=2.5 Hz, 3H), 3.32-3.26 (m, 3H), 3.13-3.02 (m, 1H), 2.19-2.13 (m, 1H), 1.56-1.45 (m, 1H). MS (ESI+) m/z 398.2 (M+H)⁺.

### Example 57

### rac-1,3-dimethyl-5-{6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridin-2-yl}pyrimidine-2,4(1H,3H)-dione

### Example 57A

### 6-methyl-2-(4,4,5,5-tetramethyl-1,3,2-dioxaborolan-2-yl)-1-tosyl-4-vinyl-1H-pyrrolo[2,3-c]pyridin-7(6H)-one

To a round bottom flask containing a stir bar was added Example 31A (1 g, 2.201 mmol), 4,4,5,5-tetramethyl-1,3,2-dioxaborolane (0.423 g, 3.30 mmol), Pd(Ph₃P)₄ (0.127 g, 0.110 mmol), and triethylamine (0.767 mL, 5.50 mmol). The flask was evacuated and backfilled three times with nitrogen and then charged with 2-methyltetrahydrofuran (20 mL). The resulting mixture was stirred at 85° C. for16 hours. The solution was poured into ethyl acetate and the organic fraction was washed with water. The organic fraction was concentrated. Purification via flash chromatography (30-80% ethyl acetate/petroleumether) afforded the title compound (530 mg, 1.108 mmol, 50.3% yield). ¹H NMR (400 MHz, CDCl₃) δ 8.32 (d, J=8.4 Hz, 2H), 7.32 (d, J=8.3 Hz, 2H), 7.04 (s, 1H), 6.55 (dd, J=17.7, 11.2 Hz, 1H), 5.57 (d, J=17.6 Hz, 1H), 5.20(d, J=11.2 Hz, 1H), 3.52 (s, 3H), 2.40 (s, 3H), 1.42 (s, 12H). MS (ESI+) m/z 455.1 (M+H)⁺.

### Example 57B

### 1,3-dimethyl-5-(6-methyl-7-oxo-1-tosyl-4-vinyl-6,7-dihydro-1H-pyrrolo[2,3-c]pyridin-2-yl)pyrimidine-2,4(1H,3H)-dione

A mixture of Example 57A (150 mg, 0.331 mmol), 5-iodo-1,3-dimethylpyrimidine-2,4(1H,3H)-dione (132 mg, 0.496 mmol) and Pd₂(dba)₃ (9.09 mg, 9.93 μmol) in tetrahydrofuran (2.5 mL) and water (0.625 mL) was added 1,3,5,7-tetramethyl-8-phenyl-2,4,6-trioxa-8-phosphaadamantane (9.67 mg, 0.033 mmol) and potassium phosphate (211 mg, 0.993 mmol). The resulting mixture was stirred at about 60° C. for about 18 hour. The mixture was filtered through diatomaceous earth and treated with 3-mercaptopropyl functionalized silica gel for 30 minutes, filtered and concentrated. The residue was purified by preparative HPLC to give the title compound (110 mg, 0.224 mmol, 67.8% yield) as a white solid. ¹H NMR (400 MHz, CDCl₃) δ 8.22 (d, J=8.4 Hz, 2H), 7.34 (s, 1H), 7.32 (d, J=8.2 Hz, 2H), 7.11 (s, 1H), 6.67 (s, 1H), 6.57 (dd, J=17.7, 11.3 Hz, 1H), 5.56 (d, J=17.6 Hz, 1H), 5.22 (d, J=11.2 Hz, 1H), 3.50 (s, 3H), 3.48 (s, 3H), 3.41 (s, 3H), 2.41 (s, 3H). MS (ESI+) m/z 467.1 (M+H)⁺.

### Example 57C

### rac-1,3-dimethyl-5-{6-methyl-1-(4-methylbenzene-1-sulfonyl)-4-[(1R,2S)-1′-methyl-2′oxo-1′,2′-dihydrospiro[cyclopropane-1,3′-indol]-2-yl]-7-oxo-6,7-dihydro-1H-pyrrolo[2,3-c]pyridin-2-yl}pyrimidine-2,4(1H,3H)-dione

A solution of 3-diazo-1-methylindolin-2-one (49.0 mg, 0.283 mmol) in dichloromethane (5 mL) was added over 1 hour via syringe pump to a solution of Example 57B (110 mg, 0.236 mmol) and rhodium(II) acetate dimer (2.084 mg, 4.72 μmol) in dichloromethane (5 mL) at room temperature. After an additional 1 hour, the reaction was concentrated under reduced pressure and the residue was purified by flash chromatography (silica gel, eluted with 50-100% ethyl acetate/petroleumether) to provide the title compound (50 mg, 0.078 mmol, 32.9% yield). MS (ESI+) m/z 612.2 (M+H)⁺.

### Example 57D

### rac-1,3-dimethyl-5-{6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridin-2-yl}pyrimidine-2,4(1H,3H)-dione

Magnesium (19.87 mg, 0.817 mmol) was added to a solution of Example 57C (50 mg, 0.082 mmol) in methanol (20 mL). The resulting suspension was sonicated for 6 hours, then diluted with aqueous NH₄Cl and extracted with ethyl acetate (3×5 mL). The combined organics were dried over MgSO₄, filtered and concentrated. The crude product was purified by preparative HPLC to give the title compound (5 mg, 10.93 μmol, 13.37% yield). ¹H NMR (400 MHz, CDCl₃) δ 11.10 (s, 1H), 7.62 (s, 1H), 7.15 (t, J=7.7 Hz, 1H), 6.89-6.83 (m, 2H), 6.73 (t, J=7.8 Hz, 1H), 6.32 (d, J=7.2 Hz, 1H), 6.14(d, J=2.2 Hz, 1H), 3.64 (s, 3H), 3.51 (s, 3H), 3.42 (s, 3H), 3.37 (s, 3H), 3.18 (t, J=8.0 Hz, 1H), 2.20 (dd, J=9.0, 4.4 Hz, 1H), 1.94 (dd, J=7.9, 4.4 Hz, 1H). MS (ESI+) m/z 458.1 (M+H)⁺.

### Example 58

### rac-N-cyclopropyl-4-[(1R,2S)-1′,2-dimethyl-2′-oxo-6′-(trifluoromethoxy)-1′,2′-dihydrospiro[cyclopropane-1,3′-indol]-2-yl]-6-methyl-7-oxo-6,7-dihydro-1H-pyrrolo[2,3-c]pyridine-2-carboxamide

### Example 58A

### 1-methyl-6-(trifluoromethoxy)indoline-2,3-dione

A solution of 6-(trifluoromethoxy)indoline-2,3-dione (1 g, 4.33 mmol, SCRC) in anhydrous N,N-dimethylformamide (20 mL) was cooled to 0° C., whereupon NaH (0.202 g, 5.06 mmol) was added in one portion. After stirring the mixture for 5 minutes, iodomethane (0.317 mL, 5.06 mmol) was added and the reaction was stirred at 0° C. for 30 minutes. The reaction mixture was then poured into saturated aqueous NH₄Cl and extracted with ethyl acetate. The combined organic fractions were washed with water and brine, dried with MgSO₄, filtered, and concentrated to give the title compound (1.061 g, 4.33 mmol, 100% yield), which was used without further purification.

### Example 58B

### 3-diazo-1-methyl-6-(trifluoromethoxy)indolin-2-one

Example 58A (1.061 g, 4.33 mmol) was added in one portion to a stirring suspension of 4-methylbenzenesulfonohydrazide (0.806 g, 4.33 mmol, SCRC) in methanol (30 mL) at room temperature. After 3 hours the reaction was concentrated to give a yellow solid. N-benzyl-N,N-diethylethanaminium chloride (0.020 g, 0.087 mmol, SCRC), dichloromethane (12 mL), and NaOH (12.00 mL, 4.00 mmol, 1M in water) were added to the solid and the resulting mixture was vigorously stirred and heated to 40° C. After 15 hours the reaction was diluted with ethyl acetate and water and the layers were separated. The organic phase was washed with water and brine, dried with MgSO₄, and concentrated under reduced pressure. The crude residue was then purified via flash chromatography (0-100% ethyl acetate/heptanes) to yield the title compound (0.390 g, 1.515 mmol, 35% yield) as solid. MS (ESI+) m/z 258.1 (M+H)⁺.

### Example 58C

### Ethyl 6-methyl-7-oxo-4-(prop-1-en-2-yl)-1-tosyl-6,7-dihydro-1H-pyrrolo[2,3-c]pyridine-2-carboxylate

Example 1F (300 mg, 0.662 mmol), tris(dibenzylideneacetone)dipalladium(0) (60.6 mg, 0.066 mmol), 1,3,5,7-tetramethyl-8-phenyl-2,4,6-trioxa-8-phosphaadamantane (19.35 mg, 0.066 mmol, SCRC), and potassium phosphate tribasic (280 mg, 1.324 mmol) were added to a round bottom flask containing a stir bar. The flask was evacuated and backfilled three times with nitrogen and then charged with dioxane (4 mL), 4,4,5,5-tetramethyl-2-(prop-1-en-2-yl)-1,3,2-dioxaborolane (222 mg, 1.324 mmol, SCRC) and water (1 mL). The resulting mixture was stirred at 70° C. for 16 hours. The solution was poured into ethyl acetate and the organic fraction was washed with water and concentrated. Purification via flash chromatography (30-80% ethyl acetate ethyl acetate/heptanes) afforded the title compound (220 mg, 0.434 mmol, 65.6% yield). MS (ESI+) m/z 415.1 (M+H)⁺.

### Example 58D

### rac-ethyl 4-[(1R,2S)-1′,2-dimethyl-2′-oxo-1′,2′-dihydrospiro[cyclopropane-1,3′-indol]-2-yl]-6-methyl-1-(4-methylbenzene-1-sulfonyl)-7-oxo-6,7-dihydro-1H-pyrrolo[2,3-c]pyridine-2-carboxylate

A solution of Example 58B (62.0 mg, 0.241 mmol) in dichloromethane (5 mL) was added over 12 hours via syringe pump to a solution of Example 58C (100 mg, 0.241 mmol) and rhodium(II) acetate dimer (1.066 mg, 2.413 mol) in dichloromethane (10 mL) at 45° C. LC/MS after an additional 6 hours showed almost complete conversion to the desired product. After an additional 6 hours the reaction was concentrated under reduced pressure and the crude residue was purified via flash chromatography (50-100% ethyl acetate/heptanes) to give the title compound (89 mg, 0.138 mmol, 57.3% yield). LC/MS (ESI+) m/z 644.4 (M+H)⁺.

### Example 58E

### rac-N-cyclopropyl-4-[(1R,2S)-1′,2-dimethyl-2′-oxo-6′-(trifluoromethoxy)-1′,2′-dihydrospiro[cyclopropane-1,3′-indol]-2-yl]-6-methyl-7-oxo-6,7-dihydro-1H-pyrrolo[2,3-c]pyridine-2-carboxamide

Aqueous NaOH (62.1 mg, 0.777 mmol, 50% in water) was added to a solution of Example 58D (50 mg, 0.078 mmol) in dioxane (5 mL) and water (1.25 mL). The reaction was then heated to 90° C. for 1 hour. After cooling to room temperature the reaction was acidified with 1M HCl and extracted three times with ethyl acetate (3 x 15 mL). The combined organic phases were washed with brine, dried with MgSO₄, and concentrated under reduced pressure. The resulting residue was taken up in N,N-dimethylformamide (5 mL) and cooled in an ice bath. HATU (44.3 mg, 0.117 mmol) and N,N-diisopropylethylamine (0.027 mL, 0.155 mmol) were added, followed after 30 minutes by cyclopropanamine (6.65 mg, 0.117 mmol, SCRC). After 15 minutes the reaction was warmed to room temperature and after an additional 1 hour the reaction was diluted with ethyl acetate. The reaction was then washed with a saturated aqueous NaHCO₃ solution and brine, dried with MgSO₄, filtered, and concentrated under reduced pressure. The crude residue was purified by preparative reverse phase HPLC (30-65% acetonitrile/water with 10 mM NH₄CO₃, Xbridge C18 column) to give the title compound (5.9 mg, 0.012 mmol, 15.18% yield). ¹H NMR (400 MHz, CDCl₃) δ 10.71 (s, 1H), 7.20 (d, J=8.1 Hz, 1H), 6.85-6.69 (m, 4H), 5.27 (s, 1H), 3.30 (s, 3H), 2.79 (s, 1H), 2.39 (s, 2H), 2.04 (d, J=2.8 Hz, 3H), 1.27 (s, 3H), 0.80 (s, 1H), 0.68 (s, 1H), 0.53 (s, 1H), 0.44 (s, 1H); MS (ESI+) m/z 501.1 (M+H)⁺.

### Biological Examples

### Bromodomain Domain Binding Assay

A time-resolved fluorescence resonance energy transfer (TR-FRET) assay was used to determine the affinities of compounds of the Examples listed in Table 1 for each bromodomain of BRD4. His-tagged first (BDI: amino acids K57-E168) and second (BDII: amino acids E352-M457) bromodomains of BRD4 were expressed and purified. An Alexa647-labeled BET-inhibitor was used as the fluorescent probe in the assay.

Synthesis of Alexa647-Labeled Bromodomain Inhibitor Compound
2-((6S,Z)-4-(4-chlorophenyl)-2,3,9-trimethyl-6H-thieno[3,2-f][1,2,4]triazolo[4,3-a][1,4]diazepin-6-yl)acetic acid. Methyl 2-((6S,Z)-4-(4-chlorophenyl)-2,3,9-trimethyl-6H-thieno[3,2-f][1,2,4]triazolo[4,3-a][1,4]diazepin-6-yl)acetate (WO 2006129623)(100.95 mg, 0.243 mmol was suspended in 1 mL methanol to which was added a freshly prepared solution of lithium hydroxide monohydrate (0.973 mL, 0.5 M, 0.487 mmol) and shaken at ambient temperature for 3 hours. The methanol was evaporated and the pH adjusted with aqueous hydrochloric acid (1 M, 0.5 mL, 0.5 mmol) and extracted four times with ethyl acetate. The combined ethyl acetate layers were dried over magnesium sulfate and evaporated to afford 2-((6S,Z)-4-(4-chlorophenyl)-2,3,9-trimethyl-6H-thieno[3,2-f][1,2,4]triazolo[4,3-a][1,4]diazepin-6-yl)acetic acid (85.3 mg, 87.0%); ESI-MS m/z=401.1 [(M+H)⁺] which was used directly in the next reaction.
N-(2-(2-(2-aminoethoxy)ethoxy)ethyl)-2((6S,Z)-4-(4-chlorophenyl)-2,3,9-trimethyl-6H-thieno[3,2-f][1,2,4]triazolo[4,3-a][1,4]diazepin-6-yl)acetamide bis(2,2,2-trifluoroacetate). 2-((6S,Z)-4-(4-chlorophenyl)-2,3,9-trimethyl-6H-thieno[3,2-f][1,2,4]triazolo[4,3-a][1,4]diazepin-6-yl)acetic acid)(85.3 mg, 0.213 mmol) was combined with 2,2′-(ethane-1,2-diylbis(oxy))diethanamine (Sigma-Aldrich, 0.315 mg, 2.13 mmol) were combined in 5 mL anhydrous dimethylformamide. (1H-benzo[d][1,2,3]triazol-1-yloxy)tripyrrolidin-1-ylphosphonium hexafluorophosphate(V) (PyBOB, CSBio, Menlo Park, Calif.; 332 mg, 0.638 mmol) was added and the reaction shaken at ambient temperature for 16 hours. The reaction was diluted to 6 mL with dimethylsulfoxide:water (9:1, v:v) and purified in two injections with time collection Waters Deltapak C18 200×25 mm column eluted with a gradient of 0.1% trifluoroacetic acid (v/v) in water and acetonitrile. The fractions containing the two purified products were lyophilized to afford N-(2-(2-(2-aminoethoxy)ethoxy)ethyl)-2-((6S,Z)-4-(4-chlorophenyl)-2,3,9-trimethyl-6H-thieno[3,2-f][1,2,4]triazolo[4,3-a][1,4]diazepin-6-yl)acetamide bis(2,2,2-trifluoroacetate) (134.4 mg, 82.3%); ESI-MS m/z=531.1 [(M+H)⁺]; 529.1 [(M−H)⁻] and (S,Z)-N,N′-(2,2′-(ethane-1,2-diylbis(oxy))bis(ethane-2,1-diyl))bis(2-((6S,Z)-4-(4-chlorophenyl)-2,3,9-trimethyl-6H-thieno[3,2-f][1,2,4]triazolo[4,3-a][1,4]diazepin-6-yl)acetamide) bis(2,2,2-trifluoroacetate) (3.0 mg, 1.5%); ESI-MS m/z=913.2 [(M+H)⁺]; 911.0 [(M−H)⁻].
N-(2-(2-(2-amido-(Alexa647)-ethoxy)ethoxy)ethyl)-2-((6S,Z)-4-(4-chlorophenyl)-2,3,9-trimethyl-6H-thieno[3,2-f][1,2,4]triazolo[4,3-a][1,4]diazepin-6-yl)acetamide(2,2,2-trifluoroacetate). N-(2-(2-(2-aminoethoxy)ethoxy)ethyl)-2-((6S,Z)-4-(4-chlorophenyl)-2,3,9-trimethyl-6H-thieno[3,2-f][1,2,4]triazolo[4,3-a][1,4]diazepin-6-yl)acetamide bis(2,2,2-trifluoroacetate) (5.4 mg, 0.0071 mmol) was combined with Alexa Fluor® 647 carboxylic Acid, succinimidyl ester (Life Technologies, Grand Island, N.Y.; 3 mg, 0.0024 mmol) were combined in 1 mL anhydrous dimethylsulfoxide containing diisopropylethylamine (1% v/v) and shaken at ambient temperature for 16 hours. The reaction was diluted to 3 mL with dimethylsulfoxide:water (9:1, v:v) and purified in one injection with time collection Waters Deltapak C18 200×25 mm column eluted with a gradient of 0.1% trifluoroacetic acid (v/v) in water and acetonitrile. The fractions containing the purified product were lyophilized to afford N-(2-(2-(2-amido-(Alexa647)-ethoxy)ethoxy)ethyl)-2-((6S,Z)-4-(4-chlorophenyl)-2,3,9-trimethyl-6H-thieno[3,2-f][1,2,4]triazolo[4,3-a][1,4]diazepin-6-yl)acetamide(2,2,2-trifluoroacetate) (1.8 mg); MALDI-MS m/z=1371.1, 1373.1 [(M+H)⁺] as a dark blue powder.

Assay

Compound dilution series were prepared in DMSO via an approximately 3-fold serial dilution. Compound dilutions were added directly into white, low-volume assay plates (Perkin Elmer Proxiplate 384 Plus# 6008280) using a Labcyte Echo in conjunction with Labcyte Access and Thermo Multidrop CombinL robotics. Compounds were then suspended in eight microliters (μL) of assay buffer (20 mM Sodium Phosphate, pH 6.0, 50 mM NaCl, 1 mM Ethylenediaminetetraacetic acid disodium salt dihydrate, 0.01% Triton X-100, 1 mM DL-Dithiothreitol) containing His-tagged bromodomain, Europium-conjugated anti-His antibody (Invitrogen PV5596) and Alexa-647-conjugated probe.

The final concentration of 1× assay mixture contained 2% DMSO, 12 nM His tagged BRD4 (BDI_K57-E168) and 100 nM probe or 4 nM His tagged BRD4 (BDII_E352-M457) and 30 nM probe, and 1 nM Europium-conjugated anti-His-tag antibody, and compound concentrations in the range of: 49.02 μM-0.61 nM or 0.98 μM 0.15 nM.

After a one-hour equilibration at room temperature, TR-FRET ratios were determined using an Envision multilabel plate reader (Ex 340, Em 495/520).

TR-FRET data were normalized to the means of 24 no-compound controls (“high”) and 8 controls containing 1 μM un-labeled probe (“low”). Percent inhibition was plotted as a function of compound concentration and the data were fit with the 4 parameter logistic equation to obtain IC50s. Inhibition constants (Kᵢ) were calculated from the IC₅₀s, probe K_{d} and probe concentration.

The mean Kᵢ values are reported in Table 1.

TABLE 1

| | TR-FRET | TR-FRET |
|---|---|---|
| | Binding | Binding |
| Compounds | Kᵢ: BRD4 | Kᵢ: BRD4 |
| of Example | (BDII_E352- | (BDI_K57- |
| No. | M457) (μM) | E168) (μM) |
| 1 | 0.0092 | 6.08 |
| 2 | 0.00477 | 5.42 |
| 3 | 0.00453 | 3.25 |
| 4 | 0.267 | >13 |
| 5 | 0.022 | 5.45 |
| 6 | 2.75 | >13.0 |
| 7 | 5.49 | >13.0 |
| 8 | 0.00543 | >13.0 |
| 9 | 0.00582 | 4.68 |
| 10 | 0.0035 | 2.05 |
| 11 | 0.0317 | 7.38 |
| 12 | 0.00365 | 4.53 |
| 13 | 0.00559 | 2.95 |
| 14 | 0.0102 | 4.8 |
| 15 | 0.00708 | 3.63 |
| 16 | 0.00669 | 9.95 |
| 17 | 0.00325 | 5.04 |
| 18 | 0.418 | >13.0 |
| 19 | 0.0153 | >13.0 |
| 20 | 0.0128 | 7.34 |
| 21 | 1.15 | >13.0 |
| 22 | 0.00353 | 4.0 |
| 23 | 0.00865 | >13 |
| 24 | 0.00598 | 4.21 |
| 25 | 1.17 | >13.0 |
| 26 | 0.00581 | 9.22 |
| 27 | 0.261 | >13.0 |
| 28 | 0.015 | 7.43 |
| 29 | 0.0137 | 4.84 |
| 30 | 0.174 | 3.44 |
| 31 | 0.0408 | 0.811 |
| 32 | 0.0912 | 2.41 |
| 33 | 0.00331 | 0.531 |
| 34 | 0.00288 | 0.441 |
| 35 | 0.198 | 1.3 |
| 36 | 0.283 | >13.0 |
| 37 | 0.00945 | 1.25 |
| 38 | 0.896 | >13.0 |
| 39 | 0.00408 | 0.885 |
| 40 | 0.45 | >13.0 |
| 41 | 0.0182 | 1.49 |
| 42 | 0.12 | 2.82 |
| 43 | 0.00763 | 1.84 |
| 44 | 0.0179 | 1.3 |
| 45 | 0.0276 | >13.0 |
| 46 | 2.23 | >13.0 |
| 47 | 0.00816 | 2.62 |
| 48 | 0.00628 | 1.42 |
| 49 | 0.0294 | 4.14 |
| 50 | 0.00944 | 1.28 |
| 51 | 0.00336 | 4.68 |
| 52 | 0.00204 | >13.0 |
| 53 | 0.0244 | 2.82 |
| 54 | 0.00968 | 1.08 |
| 55 | 0.0366 | 3.42 |
| 56 | 0.0179 | 2.76 |
| 57 | | |
| 58 | 1.7 | >13.0 |

### Efficacy of BET Inhibitors

The effect of compounds of the examples to inhibit the growth of SKM-1 xenograft tumors implanted in mice was evaluated. A suspension of cancer cells (5×10⁶ per 0.1 mL) prepared in RPMI culture medium (Invitrogen, Carlsbad, Calif.) was diluted 1:1 with a solution of Matrigel™ (BD Biosciences, Franklin Lakes, N.J.) and inoculated subcutaneously into the right hind flank of female SCID (Charles River Labs) mice. Randomization into treatment and vehicle control groups (8/group) occurred when the mean tumor volume reached approximately 250 mm³. Example 3 was formulated in 10% ethanol, 30% PEG 400, 60% Phosol-50PG. Example 34 was formulated as a nanosuspension (2% low viscosity hydroxypropylcellulose polymer/0.2% sodium dodecyl sulfate in water). Administration of compound or vehicle was initiated on the day following randomization and continued for 21-21 days. Tumors were measured twice a week throughout the treatment period using a pair of calipers and tumor volumes were calculated according to the formula V=L×W²/2 (V: volume, mm³; L: length, mm. W: width, mm). Tumor growth inhibition was calculated based on the mean tumor volume measured on the first day that the mean volume of the vehicle group exceeded 2000 mm³ according to the formula: % TGI=100−(100×(mean tumor volume of treatment group/mean tumor volume of control group))

Results are shown in Table 2.

TABLE 2

| Example | Dose | % |
|---|---|---|
| # | (mg/kg/d) | TGI |
| 3 | 5 | 27 |
| 3 | 15 | 33 |
| 3 | 45 | 43 |
| 3 | 100 | 58 |
| 3 | 150 | 52 |
| 34 | 38 | 42 |
| 34 | 75 | 74 |

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof. All publications, patents, and patent applications cited herein are hereby incorporated by reference in their entirety for all purposes.

1. A compound of formula (I) or a pharmaceutically acceptable salt thereof, 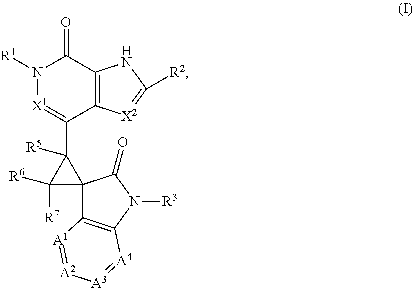
wherein
X¹ is N or C(R^{x1}) wherein R^{x1} is hydrogen, halogen, C₁-C₃ alkyl, or C₁-C₃ haloalkyl;
X² is N or CH;
R¹ is C₁-C₃ alkyl;
R² is hydrogen, G^{xi}, —C(O)OH, —NHC(O)R^{A}, or —C(O)N(H)R^{A1}; wherein
R^{A} is G^{x2}, C₁-C₆ haloalkyl, or C₁-C₆ alkyl; wherein the C₁-C₆ haloalkyl and the C₁-C₆ alkyl are optionally substituted with 1 or 2 substituents independently selected from the group consisting of —OR^{B}, —CN, G^{x2}, and —N(R^{B})₂;
R^{A1} is hydrogen, G^{x2}, C₁-C₆ haloalkyl, or C₁-C₆ alkyl; wherein the C₁-C₆ haloalkyl and the C₁-C₆ alkyl are optionally substituted with 1 or 2 substituents independently selected from the group consisting of —OR^{B}, —CN, G^{x2}, and —N(R^{B})₂;
G^{x1} and G^{x2}, at each occurrence, are each independently C₆-C₁₀ aryl, 5-11 membered heteroaryl, C₃-C₇ monocyclic cycloalkyl, C₄-C₆ monocyclic cycloalkenyl, or 4-11 membered heterocycle; each G^{x1} and G^{x2} are optionally substituted with 1, 2, or 3 substituents independently selected from the group consisting of cyclopropyl and R^{s}; wherein the cyclopropyl is optionally substituted with 1, 2, or 3 substituents independently selected from the group consisting of halogen, C₁-C₆ haloalkyl, and C₁-C₆ alkyl;
each R^{B} is independently hydrogen, C₁-C₆ alkyl, or C₁-C₆ haloalkyl;
R³ is hydrogen, C₂-C₆ alkenyl, C₂-C₆ alkynyl, C₃-C₇ monocyclic cycloalkyl, C₁-C₆ alkyl, or C₁-C₆ haloalkyl, wherein the C₁-C₆ alkyl and the C₁-C₆ haloalkyl are each optionally substituted with 1 or 2 substituents independently selected from the group consisting of —OR^{a}, —CN, —N(R^{a})₂, and phenyl; wherein the C₃-C₇ monocyclic cycloalkyl and the phenyl are each optionally substituted with 1, 2, or 3 independently selected R^{t} groups;
R^{a}, at each occurrence, is independently hydrogen, C₁-C₆ alkyl, or C₁-C₆ haloalkyl;
A¹, A², A³, and A⁴ are CR⁴ or
one or two of A¹, A², A³, and A⁴ are N, and the others are CR⁴;
R⁴, at each occurrence, is independently hydrogen, C₁-C₆ alkyl, C₂-C₆ alkenyl, C₂-C₆ alkynyl, halogen, C₁-C₆ haloalkyl, —CN,
NO₂, —OR^{x}, —OC(O)R^{y}, —OC(O)N(R^{x})₂, —S(O)₂R^{x}, —S(O)₂N(R^{x})₂, —C(O)R^{x}, —C(O)OR^{x}, —C(O)N(R^{x})₂, —C(O)N(R^{x})S(O)₂R^{y}, —N(R^{x})₂, —N(R^{x})C(O)R^{y}, —N(R^{x})S(O)₂R^{y}, —N(R^{x})C(O)O(R³), —N(R^{x})C(O)N(R^{x})₂, —N(R^{x})S(O)₂N(R^{x})₂, —(C₁-C₆ alkylenyl)-CN, —(C₁-C₆ alkylenyl)—OR^{x}, —(C₁-C₆ alkylenyl)—OC(O)R^{y}, —(C₁-C₆ alkylenyl)—OC(O)N(R^{x})₂, —(C₁-C₆ alkylenyl)-SR^{x}, —(C₁-C₆ alkylenyl)-S(O)₂R^{x}, —(C₁-C₆ alkylenyl)—S(O)₂N(R^{x})₂, —(C₁-C₆ alkylenyl)—C(O)R^{x}, —(C₁-C₆ alkylenyl)—C(O)OR^{x}, —(C₁-C₆ alkylenyl)—C(O)N(R^{x})₂, —(C₁-C₆ alkylenyl)-C(O)N(R^{x})S(O)₂R^{y}, —(C₁-C₆ alkylenyl)—N(R^{x})₂, —(C₁-C₆ alkylenyl)-N(R^{x})C(O)R^{y}, —(C₁-C₆ alkylenyl)—N(R^{x})S(O)₂R^{y}, —(C₁-C₆ alkylenyl)-N(R^{x})C(O)O(R³), —(C₁-C₆ alkylenyl)—N(R^{x})C(O)N(R^{x})₂, or —(C₁-C₆ alkylenyl)-N(R^{x})S(O)₂N(R^{x})₂;
R^{s} and R^{t}, at each occurrence, are each independently C₁-C₆ alkyl, C₂-C₆ alkenyl, C₂-C₆ alkynyl, halogen, C₁-C₆ haloalkyl, oxo, —CN,
NO₂, —OR^{x}, —OC(O)R^{y}, —OC(O)N(R^{x})₂, —SR^{x}, —S(O)₂R^{x}, —S(O)₂N(R^{x})₂, —C(O)R^{x}, —C(O)OR^{x}, —C(O)N(R^{x})₂, —C(O)N(R^{x})S(O)₂R^{y}, —N(R^{x})₂, —N(R^{x})C(O)R^{y}, —N(R^{x})S(O)₂R^{y}, —N(R^{x})C(O)O(R³), —N(R^{x})C(O)N(R^{x})₂, —N(R^{x})S(O)₂N(R^{x})₂, —(C₁-C₆ alkylenyl)-CN, —(C₁-C₆ alkylenyl)—OR^{x}, —(C₁-C₆ alkylenyl)—OC(O)R^{y}, —(C₁-C₆ alkylenyl)—OC(O)N(R^{x})₂, —(C₁-C₆ alkylenyl)-SR^{x}, —(C₁-C₆ alkylenyl)-S(O)₂R^{x}, —(C₁-C₆ alkylenyl)—S(O)₂N(R^{x})₂, —(C₁-C₆ alkylenyl)—C(O)R^{x}, —(C₁-C₆ alkylenyl)—C(O)OR^{x}, —(C₁-C₆ alkylenyl)—C(O)N(R^{x})₂, —(C₁-C₆ alkylenyl)-C(O)N(R^{x})S(O)₂R^{y}, —(C₁-C₆ alkylenyl)—N(R^{x})₂, —(C₁-C₆ alkylenyl)-N(R^{x})C(O)R^{y}, —(C₁-C₆ alkylenyl)—N(R^{x})S(O)₂R^{y}, —(C₁-C₆ alkylenyl)-N(F^{x})C(O)O(R³), —(C₁-C₆ alkylenyl)—N(R^{x})C(O)N(R^{x})₂, or —(C₁-C₆ alkylenyl)-N(R^{x})S(O)₂N(R^{x})₂;
R⁵ is hydrogen, halogen, C₁-C₃ alkyl, C₁-C₃ haloalkyl, or —(C₁-C₃ alkylenyl)—OR^{x};
R⁶ is hydrogen, halogen, C₁-C₆ alkyl, or C₁-C₆ haloalkyl;
R⁷ is hydrogen, halogen, C₁-C₆ alkyl, C₁-C₆ haloalkyl, —(C₁-C₆ alkylenyl)—OR^{x}, —(C₁-C₆ alkylenyl)—S(O)₂R^{x}, or —(C₁-C₆ alkylenyl)—S(O)₂N(R^{x})₂;
R^{x}, at each occurrence, is independently hydrogen, C₁-C₆ alkyl, or C₁-C₆ haloalkyl; and
R^{y}, at each occurrence, is independently C₁-C₆ alkyl or C₁-C₆ haloalkyl.

2. The compound of claim 1 or a pharmaceutically acceptable salt thereof, wherein
R¹ is CH₃;
X¹ is C(R^{x1}); and
X² is CH.

3. The compound of claim 1 or a pharmaceutically acceptable salt thereof, wherein
R¹ is CH₃;
A¹, A², A³, and A⁴ are CR⁴; and
each R⁴ is independently hydrogen, C₁-C₆ alkyl, halogen, C₁-C₆ haloalkyl, —CN, NO₂, or —OR^{x}.

4. The compound of claim 1 or a pharmaceutically acceptable salt thereof, wherein
R² is hydrogen, G^{x1}, —C(O)OH, or —C(O)N(H)R^{A1}.

5. The compound of claim 1 or a pharmaceutically acceptable salt thereof, wherein R² is G^{x1} or —C(O)N(H)R^{A1}.

6. The compound of claim 5 or a pharmaceutically acceptable salt thereof, wherein
R^{A1} is G^{x2}, C₁-C₆ alkyl, or C₁-C₆ haloalkyl.

7. The compound of claim 1 or a pharmaceutically acceptable salt thereof, wherein
R³ is hydrogen, C₃-C₇ monocyclic cycloalkyl, C₁-C₆ alkyl, or C₁-C₆ haloalkyl,
wherein the C₁-C₆ alkyl is optionally substituted with 1 or 2 substituents independently selected from the group consisting of —OR^{a} and phenyl;
wherein the C₃-C₇ monocyclic cycloalkyl and the phenyl are each optionally substituted with 1, 2, or 3 independently selected R^{t} groups.

8. The compound of claim 1 or a pharmaceutically acceptable salt thereof, wherein
R³ is C₁-C₆ alkyl or C₁-C₆ haloalkyl.

9. The compound of claim 1 or a pharmaceutically acceptable salt thereof, wherein
R¹ is CH₃;
X¹ is C(R^{x1});
X² is CH;
A¹, A², A³, and A⁴ are CR⁴; and
each R⁴ is independently hydrogen, C₁-C₆ alkyl, halogen, C₁-C₆ haloalkyl, —CN, NO₂, or —OR^{x}.

10. The compound of claim 1 or a pharmaceutically acceptable salt thereof, wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x}; and
R² is hydrogen, G^{x1}, —C(O)OH, or —C(O)N(H)R^{A1}.

11. The compound of claim 1 or a pharmaceutically acceptable salt thereof, wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x}; and
R² is G^{x1} wherein G^{x1} is monocyclic heteroaryl or C₃-C₇ monocyclic cycloalkyl; each of which is optionally substituted with 1, 2, or 3 independently selected R^{s} groups.

12. The compound of claim 1 or a pharmaceutically acceptable salt thereof, wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x}; and
R² is —C(O)N(H)R^{A1} wherein R^{A1} is C₁-C₆ alkyl or G^{x2}.

13. The compound of claim 1 or a pharmaceutically acceptable salt thereof, wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x}; and
R³ is hydrogen, C₃-C₇ monocyclic cycloalkyl, C₁-C₆ alkyl, or C₁-C₆ haloalkyl, wherein the C₁-C₆ alkyl is optionally substituted with one substituent selected from the group consisting of —OR^{a} and phenyl; wherein the C₃-C₇ monocyclic cycloalkyl and the phenyl are each optionally substituted with 1, 2, or 3 independently selected R^{t} groups.

14. The compound of claim 1 or a pharmaceutically acceptable salt thereof, wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x}; and
R³ is C₁-C₆ alkyl or C₁-C₆ haloalkyl.

15. The compound of claim 1, or a pharmaceutically acceptable salt thereof, wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x};
R² is G^{x1} or C(O)N(H)R^{A1}; and
R³ is C₁-C₆ alkyl.

16. The compound of claim 1, or a pharmaceutically acceptable salt thereof, wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x};
R² is G^{x1} wherein W¹ is monocyclic heteroaryl which is optionally substituted with 1, 2, or 3 independently selected R^{s} groups; and
R³ is C₁-C₆ alkyl.

17. The compound of claim 1, or a pharmaceutically acceptable salt thereof, wherein
R¹ is CH₃;
X¹ is C(R^{x1});
X² is CH;
A¹, A², A³, and A⁴ are CR⁴;
each R⁴ is independently hydrogen, C₁-C₆ alkyl, halogen, C₁-C₆ haloalkyl, —CN, NO₂, or —OR^{x};
R² is —C(O)N(H)R^{A1} wherein R^{A1} is G^{x2};
G^{x2} is C₃-C₇ monocyclic cycloalkyl optionally substituted with 1, 2, or 3 independently selected R^{s} groups; and
R³ is C₁-C₆ alkyl.

18. The compound of claim 1 of formula (I-a), or a pharmaceutically acceptable salt thereof, 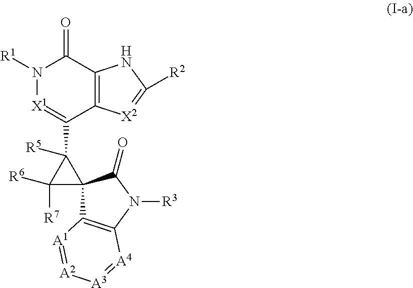
wherein R¹, R², R³, R⁵, R⁶, R⁷, X¹, X², A¹, A², A³, and A⁴ are as set forth in claim 1.

19. The compound of claim 1 or a pharmaceutically acceptable salt thereof, wherein the compound is selected from the group consisting of
rac-N-ethyl-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide;
rac-N-cyclopropyl-6-methyl-4-[(1R,2S)-1′-methyl-2′oxo-1′,2′-dihydrospiro[cyclopropane-1,3′-indol]-2-yl]-7-oxo-6,7-dihydro-1H-pyrrolo[2,3-c]pyridine-2-carboxamide;
N-cyclopropyl-6-methyl-4-[(1S,2R)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide;
N-cyclopropyl-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide;
rac-N-(3,3-difluorocyclobutyl)-6-methyl-4-[(1R,2S)-1′-methyl-2′oxo-1′,2′-dihydrospiro[cyclopropane-1,3′-indol]-2-yl]-7-oxo-6,7-dihydro-1H-pyrrolo[2,3-c]pyridine-2-carboxamide;
6-methyl-4-[(1S,2R)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxylic acid;
6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxylic acid;
rac-6-methyl-4-[(1R,2S)-1′-methyl-2′-oxo-1′,2′-dihydrospiro[cyclopropane-1,3′-indol]-2-yl]-7-oxo-N-phenyl-6,7-dihydro-1H-pyrrolo[2,3-c]pyridine-2-carboxamide;
rac-N-(4-methoxyphenyl)-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide;
rac-N-[3-(hydroxymethyl)phenyl]-6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridine-2-carboxamide;
rac-N-cyclopropyl-4-[(1R,2S)-6′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;
rac-4-[(1R,2S)-6′-chloro-′-methyl-2′-oxo-1′,2′-dihydrospiro[cyclopropane-1,3′-indol]-2-yl]-N-cyclopropyl-6-methyl-7-oxo-6,7-dihydro-1H-pyrrolo[2,3-c]pyridine-2-carboxamide;
rac-N-cyclopropyl-4-[(1R,2S)-1′,6′-dimethyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;
rac-N-cyclopropyl-4-[(1R,2S)-6′-methoxy-′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;
rac-N-cyclopropyl-6-methyl-4-[(1R,2S)-1′-methyl-2′-oxo-6′-(trifluoromethoxy)-1′,2′-dihydrospiro[cyclopropane-1,3′-indol]-2-yl]-7-oxo-6,7-dihydro-1H-pyrrolo[2,3-c]pyridine-2-carboxamide;
rac-N-cyclopropyl-4-[(1R,2S)-5′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;
N-cyclopropyl-4-[(1S,2R)-5′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;
N-cyclopropyl-4-[(1R,2S)-5′-fluoro-1′-methyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;
rac-N-cyclopropyl-6-methyl-7-oxo-4-[(1R,2S)-2′-oxo-1′,2′-dihydrospiro[cyclopropane-1,3′-indol]-2-yl]-6,7-dihydro-1H-pyrrolo[2,3-c]pyridine-2-carboxamide;
N-cyclopropyl-6-methyl-7-oxo-4-[(1S,2R)-2′-oxo-1′,2′-dihydrospiro[cyclopropane-1,3′-indol]-2-yl]-6,7-dihydro-1H-pyrrolo[2,3-c]pyridine-2-carboxamide;
N-cyclopropyl-6-methyl-7-oxo-4-[(1R,2S)-2′-oxo-1′,2′-dihydrospiro[cyclopropane-1,3′-indol]-2-yl]-6,7-dihydro-1H-pyrrolo[2,3-c]pyridine-2-carboxamide;
rac-N-cyclopropyl-4-[(1R,2S)-1′-ethyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;
rac-N-cyclopropyl -6-methyl-7-oxo-4-[(1R,2S)-2′-oxo-′-(2,2,2-trifluoroethyl)-1′,2′-dihydrospiro[cyclopropane-1,3′-indol]-2-yl]-6,7-dihydro-1H-pyrrolo[2,3-c]pyridine-2-carboxamide;
N-cyclopropyl-6-methyl-7-oxo-4-[(1S,2R)-2′oxo-′-(2,2,2-trifluoroethyl)-1′,2′-dihydrospiro[cyclopropane-1,3′-indol]-2-yl]-6,7-dihydro-1H-pyrrolo[2,3-c]pyridine-2-carboxamide;
N-cyclopropyl-6-methyl-7-oxo-4-[(1R,2S)-2′-oxo-1′-(2,2,2-trifluoroethyl)-1′,2′-dihydrospiro[cyclopropane-1,3′-indol]-2-yl]-6,7-dihydro-1H-pyrrolo[2,3-c]pyridine-2-carboxamide;
rac-N-cyclopropyl-4-[(1R,2S)-1′-cyclopropyl-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;
rac-N-cyclopropyl -4-[(1R,2S)-1′-(2-methoxyethyl)-2′-oxo-1′,2′-dihydrospiro[cyclopropane-1,3′-indol]-2-yl]-6-methyl-7-oxo-6,7-dihydro-1H-pyrrolo[2,3-c]pyridine-2-carboxamide;
rac-4-{(1R,2S)-1′-[(4-aminophenyl)methyl]-2′-oxo-1′,2′-dihydrospiro[cyclopropane-1,3′-indol]-2-yl}-N-cyclopropyl-6-methyl-7-oxo-6,7-dihydro-1H-pyrrolo[2,3-c]pyridine-2-carboxamide;
rac-4-[(1R,2S)-7′-bromo-1′-methyl-2′-oxo-1′,2′-dihydrospiro[cyclopropane-1,3′-indol]-2-yl]-N-cyclopropyl-6-methyl-7-oxo-6,7-dihydro-1H-pyrrolo[2,3-c]pyridine-2-carboxamide;
rac-(1R,2S)-1′-methyl-2-(6-methyl-7-oxo-6,7-dihydro-1H-pyrrolo[2,3-c]pyridin-4-yl)spiro[cyclopropane-1,3′-indol]-2′(1H)-one;
rac-(1R,2S)-2-(2-cyclopropyl-6-methyl-7-oxo-6,7-dihydro-1H-pyrrolo[2,3-c]pyridin-4-yl)-1′-methylspiro[cyclopropane-1,3′-indol]-2′(1H)-one;
rac-tert-butyl 3-{6-methyl-4-[(1R,2S)-1′-methyl-2′oxo-1′,2′-dihydrospiro[cyclopropane-1,3′-indol]-2-yl]-7-oxo-6,7-dihydro-1H-pyrrolo[2,3-c]pyridin-2-yl}azetidine-1-carboxylate;
rac-(1R,2S)-1′methyl-2-[6-methyl-2-(1-methyl-1H-pyrazol-4-yl)-7-oxo-6,7-dihydro-1H-pyrrolo[2,3-c]pyridin-4-yl]spiro[cyclopropane-1,3′-indol]-2′(1H)-one;
(1S,2R)-1′-methyl-2-[6-methyl-2-(1-methyl-1H-pyrazol-4-yl)-7-oxo-6,7-dihydro-1H-pyrrolo[2,3-c]pyridin-4-yl]spiro[cyclopropane-1,3′-indol]-2′(1H)-one;
(1R,2S)-1′-methyl-2-[6-methyl-2-(1-methyl-1H-pyrazol-4-yl)-7-oxo-6,7-dihydro-1H-pyrrolo[2,3-c]pyridin-4-yl]spiro[cyclopropane-1,3′-indol]-2′(1H)-one;
rac-(1R,2S)-2-[2-(1,3-dimethyl-1H-pyrazol-4-yl)-6-methyl-7-oxo-6,7-dihydro-1H-pyrrolo[2,3-c]pyridin-4-yl]-1′-methylspiro[cyclopropane-1,3′-indol]-2′(1H)-one;
rac-(1R,2S)-1′-methyl-2-[6-methyl-7-oxo-2-(1H-pyrazol-3-yl)-6,7-dihydro-1H-pyrrolo[2,3-c]pyridin-4-yl]spiro[cyclopropane-1,3′-indol]-2′(1H)-one;
rac-(1R,2R)-1′-methyl-2-[6-methyl-7-oxo-2-(1H-pyrazol-3-yl)-6,7-dihydro-1H-pyrrolo[2,3-c]pyridin-4-yl]spiro[cyclopropane-1,3′-indol]-2′(1H)-one;
rac-(1R,2S)-1′-methyl-2-[6-methyl-2-(3-methyl-1,2-oxazol-5-yl)-7-oxo-6,7-dihydro-1H-pyrrolo[2,3-c]pyridin-4-yl]spiro[cyclopropane-1,3′-indol]-2′(1H)-one;
rac-(1R,2R)-1′-methyl-2-[6-methyl-2-(3-methyl-1,2-oxazol-5-yl)-7-oxo-6,7-dihydro-1H-pyrrolo[2,3-c]pyridin-4-yl]spiro[cyclopropane-1,3′-indol]-2′(1H)-one;
rac-(1R,2S)-1′-methyl-2-[6-methyl-2-(1-methyl-1H-imidazol-2-yl)-7-oxo-6,7-dihydro-1H-pyrrolo[2,3-c]pyridin-4-yl]spiro[cyclopropane-1,3′-indol]-2′(1H)-one;
rac-(1R,2S)-1′-methyl-2-[6-methyl-2-(1,3-oxazol-2-yl)-7-oxo-6,7-dihydro-1H-pyrrolo[2,3-c]pyridin-4-yl]spiro[cyclopropane-1,3′-indol]-2′(1H)-one;
rac-(1R,2S)-1′-methyl-2-[6-methyl-2-(1-methyl-1H-imidazol-4-yl)-7-oxo-6,7-dihydro-1H-pyrrolo[2,3-c]pyridin-4-yl]spiro[cyclopropane-1,3′-indol]-2′(1H)-one;
rac-(1R,2S)-1′-methyl-2-[6-methyl-2-(1-methyl-1H-imidazol-5-yl)-7-oxo-6,7-dihydro-1H-pyrrolo[2,3-c]pyridin-4-yl]spiro[cyclopropane-1,3′-indol]-2′(1H)-one;
rac-(1R,2S)-1′-methyl-2-[6-methyl-7-oxo-2-(pyridin-2-yl)-6,7-dihydro-1H-pyrrolo[2,3-c]pyridin-4-yl]spiro[cyclopropane-1,3′-indol]-2′(1H)-one;
rac-(1R,2S)-1′-methyl-2-[6-methyl-2-(4-methylpyridin-2-yl)-7-oxo-6,7-dihydro-1H-pyrrolo[2,3-c]pyridin-4-yl]spiro[cyclopropane-1,3′-indol]-2′(1H)-one;
rac-(1R,2S)-1′-methyl-2-[6-methyl-7-oxo-2-(pyridin-3-yl)-6,7-dihydro-1H-pyrrolo[2,3-c]pyridin-4-yl]spiro[cyclopropane-1,3′-indol]-2′(1H)-one;
(1S,2R)-1′-methyl-2-[6-methyl-7-oxo-2-(pyridin-3-yl)-6,7-dihydro-1H-pyrrolo [2,3-c]pyridin-4-yl]spiro[cyclopropane-1,3′-indol]-2′(1H)-one;
(1R,2S)-1′-methyl-2-[6-methyl-7-oxo-2-(pyridin-3-yl)-6,7-dihydro-1H-pyrrolo [2,3-c]pyridin-4-yl]spiro[cyclopropane-1,3′-indol]-2′(1H)-one;
rac-(1R,2S)-1′-methyl-2-[6-methyl-7-oxo-2-(pyridin-4-yl)-6,7-dihydro-1H-pyrrolo[2,3-c]pyridin-4-yl]spiro[cyclopropane-1,3′-indol]-2′(1H)-one;
rac-(1R,2S)-1′-methyl-2-{6-methyl-7-oxo-2-[2-(trifluoromethyl)pyridin-4-yl]-6,7-dihydro-1H-pyrrolo[2,3-c]pyridin-4-yl}spiro[cyclopropane-1,3′-indol]-2′(1H)-one;
(1S,2R)-1′-methyl-2-{6-methyl-7-oxo-2-[2-(trifluoromethyl)pyridin-4-yl]-6,7-dihydro-1H-pyrrolo[2,3-c]pyridin-4-yl}spiro[cyclopropane-1,3′-indol]-2′(1H)-one;
(1R,2S)-1′-methyl-2-{6-methyl-7-oxo-2-[2-(trifluoromethyl)pyridin-4-yl]-6,7-dihydro-1H-pyrrolo[2,3-c]pyridin-4-yl}spiro[cyclopropane-1,3′-indol]-2′(1H)-one;
rac-(1R,2S)-1′-methyl-2-[6-methyl-7-oxo-2-(pyrimidin-5-yl)-6,7-dihydro-1H-pyrrolo[2,3-c]pyridin-4-yl]spiro[cyclopropane-1,3′-indol]-2′(1H)-one;
rac-(1R,2S)-1′-methyl-2-[6-methyl-7-oxo-2-(pyrimidin-4-yl)-6,7-dihydro-1H-pyrrolo[2,3-c]pyridin-4-yl]spiro[cyclopropane-1,3′-indol]-2′(1H)-one;
rac-(1R,2S)-1′-methyl-2-[6-methyl-7-oxo-2-(pyridazin-4-yl)-6,7-dihydro-1H-pyrrolo[2,3-c]pyridin-4-yl]spiro[cyclopropane-1,3′-indol]-2′(1H)-one;
rac-1,3-dimethyl-5-{6-methyl-4-[(1R,2S)-1′-methyl-2′-oxo-1′,2′-dihydrospiro[cyclopropane-1,3′-indol]-2-yl]-7-oxo-6,7-dihydro-1H-pyrrolo[2,3-c]pyridin-2-yl}pyrimidine-2,4(1H,3H)-dione; and
rac-N-cyclopropyl-4-[(1R,2S)-1′,2-dimethyl-2′-oxo-6′-(trifluoromethoxy)-1′,2′-dihydrospiro[cyclopropane-1,3′-indol]-2-yl]-6-methyl-7-oxo-6,7-dihydro-1H-pyrrolo[2,3-c]pyridine-2-carboxamide.

20. A pharmaceutical composition comprising a therapeutically effective amount of a compound of formula (I) according to claim 1, or a pharmaceutically acceptable salt thereof, in combination with a pharmaceutically acceptable carrier.

21. A method for treating cancer in a subject comprising administering a therapeutically effective amount of a compound of formula (I) according to claim 1 or a pharmaceutically acceptable salt thereof, to a subject in need thereof.

22. The method of claim 21 wherein the cancer is selected from the group consisting of:
acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute t-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, glioblastoma, gliosarcoma, heavy chain disease, hemangioblastoma, hepatoma, hepatocellular cancer, hormone insensitive prostate cancer, leiomyosarcoma, leukemia, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, medullary carcinoma, medulloblastoma, melanoma, meningioma, mesothelioma, multiple myeloma, myelogenous leukemia, myeloma, myxosarcoma, neuroblastoma, NUT midline carcinoma (NMC), non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenström's macroglobulinemia, testicular tumors, uterine cancer, and Wilms' tumor.

23. A method for treating a disease or condition in a subject comprising administering a therapeutically effective amount of a compound of formula (I) according to claim 1 or a pharmaceutically acceptable salt thereof, to a subject in need thereof, wherein said disease or condition is selected from the group consisting of Addison's disease, acute gout, ankylosing spondylitis, asthma, atherosclerosis, Behcet's disease, bullous skin diseases, chronic obstructive pulmonary disease (COPD), Crohn's disease, dermatitis, eczema, giant cell arteritis, glomerulonephritis, hepatitis, hypophysitis, inflammatory bowel disease, Kawasaki disease, lupus nephritis, multiple sclerosis, myocarditis, myositis, nephritis, organ transplant rejection, osteoarthritis, pancreatitis, pericarditis, polyarteritis nodosa, pneumonitis, primary biliary cirrhosis, psoriasis, psoriatic arthritis, rheumatoid arthritis, scleritis, sclerosing cholangitis, sepsis systemic lupus erythematosus, Takayasu's arteritis, toxic shock, thyroiditis, type I diabetes, ulcerative colitis, uveitis, vitiligo, vasculitis, and Wegener's granulomatosis.

24. A method for treating an acquired immunodeficiency syndrome (AIDS) in a subject comprising administering a therapeutically effective amount of a compound of formula (I) according to claim 1 or a pharmaceutically acceptable salt thereof, to a subject in need thereof.

25. A method for treating a disease or condition in a subject comprising administering a therapeutically effective amount of a compound of formula (I) according to claim 1 or a pharmaceutically acceptable salt thereof, to a subject in need thereof, wherein said disease or condition is selected from the group consisting of: obesity, dyslipidemia, hypercholesterolemia, Alzheimer's disease, metabolic syndrome, hepatic steatosis, type II diabetes, insulin resistance, diabetic retinopathy, and diabetic neuropathy.

26. A method of contraception in a male subject comprising administering a therapeutically effective amount of a compound of formula (I) according to claim 1 or a pharmaceutically acceptable acceptable salt thereof, to a subject in need thereof.

27. A method for treating an acute kidney disease or condition in a subject comprising administering a therapeutically effective amount of a compound of formula (I) according to claim 1 or a pharmaceutically acceptable salt thereof, to a subject in need thereof, wherein said acute kidney disease or condition is selected from the group consisting of: ischemia-reperfusion induced kidney disease, cardiac and major surgery induced kidney disease, percutaneous coronary intervention induced kidney disease, radio-contrast agent induced kidney disease, sepsis induced kidney disease, pneumonia induced kidney disease, and drug toxicity induced kidney disease.

28. A method of treating a chronic kidney disease or condition in a subject comprising administering a therapeutically effective amount of a compound of formula (I) according to claim 1 or a pharmaceutically acceptable salt thereof, to a subject in need thereof, wherein said disease or condition is selected from the group consisting of: diabetic nephropathy, hypertensive nephropathy, HIV-associated nephropathy, glomerulonephritis, lupus nephritis, IgA nephropathy, focal segmental glomerulosclerosis, membranous glomerulonephritis, minimal change disease, polycystic kidney disease and tubular interstitial nephritis.
